SEQUENCE LISTING (111 sequences)

SEQ ID NO: 1 - protein, 374 aa, Homo sapiens
MSGESMNFSDVFDSSEDYFVSVNTSYYSVDSEMLLCSLQEVRQFSRLFVPIAYSLICVFGLLGNILVVITFAFYKKARSMTDVYLLNMAIADILFVLTLPFWAVSHATGAWVFSNATCKLLKGIYAINFNCGMLLLTCISMDRYIAIVQATKSFRLRSRTLPRSKIICLVVWGLSVIISSSTFVFNQKYNTQGSDVCEPKYQTVSEPIRWKLLMLGLELLFGFFIPLMFMIFCYTFIVKTLVQAQNSKRHKAIRVIIAVVLVFLACQIPHNMVLLVTAANLGKMNRSCQSEKLIGYTKTVTEVLAFLHCCLNPVLYAFIGQKFRNYFLKILKDLWCVRRKYKSSGFSCAGRYSENISRQTSETADNDNASSFTM

SEQ ID NO: 2 - protein, 28 aa, Homo sapiens
MSGESMNFSDVFDSSEDYFASVNTSYYT

SEQ ID NO: 3 - protein, 8 aa, Mus musculus
GFTFSDYY

SEQ ID NO: 4 - protein, 8 aa, Mus musculus
ITNGDGRT

SEQ ID NO: 5 - protein, 12 aa, Mus musculus
TSPPLGGAWFGY

SEQ ID NO: 6 - protein, 8 aa, Mus musculus
GFSFSDYY

SEQ ID NO: 7 - protein, 8 aa, Mus musculus
ITNGGGRT

SEQ ID NO: 8 - protein, 8 aa, Mus musculus
ITNGAGRT

SEQ ID NO: 9 - protein, 8 aa, Mus musculus
ITNGAGRT

SEQ ID NO: 10 - protein, 12 aa, Mus musculus
ASPPLGGAWFAY

SEQ ID NO: 11 - protein, 8 aa, Mus musculus
GFPFSDYY

SEQ ID NO: 12 - protein, 8 aa, Mus musculus
ITNGVGRT

SEQ ID NO: 13 - protein, 12 aa, Mus musculus
TSPPLGGAWFAY

SEQ ID NO: 14 - protein, 8 aa, Mus musculus
EYTFKSFG

SEQ ID NO: 15 - protein, 8 aa, Mus musculus
IYPRSGNT

SEQ ID NO: 16 - protein, 11 aa, Mus musculus
ARSPYDGYFDY

SEQ ID NO: 17 - protein, 11 aa, Mus musculus
QSIVHSNGNTY

SEQ ID NO: 18 - protein, 3 aa, Mus musculus
KVS

SEQ ID NO: 19 - protein, 9 aa, Mus musculus
FQGSHVPLT

SEQ ID NO: 20 - protein, 11 aa, Mus musculus
QSLVHSNGNTY

SEQ ID NO: 21 - protein, 11 aa, Mus musculus
QSLLHINGNTY

SEQ ID NO: 22 - protein, 3 aa, Mus musculus
RVS

SEQ ID NO: 23 - protein, 9 aa, Mus musculus
SQSTHVPRT

SEQ ID NO: 24 - protein, 25 aa, Mus musculus
EVNLVESGGGLVQPGGSLKLSCAAS

SEQ ID NO: 25 - protein, 25 aa, Mus musculus
EVNLVESGGGLVQPGGSLKLSCEAS

SEQ ID NO: 26 - protein, 25 aa, Mus musculus
EVKLVESGGGLVQPGGSLKLSCAAS

SEQ ID NO: 27 - protein, 25 aa, Mus musculus
QDQLQQSGVALARPGASVKLSCKAS

SEQ ID NO: 28 - protein, 17 aa, Mus musculus
MYWVRQTPEKRLEWVTY

SEQ ID NO: 29 - protein, 17 aa, Mus musculus
LYWVRQTPEKRLEWVTY

SEQ ID NO: 30 - protein, 17 aa, Mus musculus
LYWVRQTPEKRLEWVAY

SEQ ID NO: 31 - protein, 17 aa, Mus musculus
LGWVKQRPGQGLEWIGE

SEQ ID NO: 32 - protein, 38 aa, Mus musculus
YYSDTVRGRFTISRDNAKNTLYLQMSRLKSEDTAMYYC

SEQ ID NO: 33 - protein, 38 aa, Mus musculus
YYSDTIRGRFTISRDNARNTLYLQMSRLKSEDTAMYYC

SEQ ID NO: 34 - protein, 38 aa, Mus musculus
YYSDSVKGRFTISRDNAKNTLYLQMSRLKSEDTSMYYC

SEQ ID NO: 35 - protein, 38 aa, Mus musculus
YYNEKVKGKVRLTADKSSNSVYMEFRSLTSEDSAVYFC

SEQ ID NO: 36 - protein, 10 aa, Mus musculus
WGQGTLVTVS

SEQ ID NO: 37 - protein, 10 aa, Mus musculus
WGQGTTLTVS

SEQ ID NO: 38 - protein, 26 aa, Mus musculus
DVLMTQTPLSLPVSLGDQASISCRSS

SEQ ID NO: 39 - protein, 26 aa, Mus musculus
DVSMTQTPLSLPVSLGDQASISCRSS

SEQ ID NO: 40 - protein, 26 aa, Mus musculus
DVVMTHSPLSLPVSLGDQASISCRSS

SEQ ID NO: 41 - protein, 17 aa, Mus musculus
LEWYLQKPGQSPKLLIY

SEQ ID NO: 42 - protein, 17 aa, Mus musculus
LHWYLQKPGQSPKLLIY

SEQ ID NO: 43 - protein, 36 aa, Mus musculus
KRFSGVPDRFSGSGSGTDFTLKISRVEAEDLGVYYC

SEQ ID NO: 44 - protein, 36 aa, Mus musculus
KRFSGVPDRFSGSGSGTDFTLKISRVGAEDLGVYYC

SEQ ID NO: 45 - protein, 36 aa, Mus musculus
NRLSGVPDRFSGSGSGTDFTLKISRVEAEDLGVYFC

SEQ ID NO: 46 - protein, 11 aa, Mus musculus
FGAGTKLELKR

SEQ ID NO: 47 - protein, 11 aa, Mus musculus
FGGGTKLEIKR

SEQ ID NO: 48 - protein, 118 aa, Mus musculus
EVNLVESGGGLVQPGGSLKLSCAASGFTFSDYYMYWVRQTPEKRLEWVTYITNGDGRTYYSDTVRGRFTISRDNAKNTLYLQMSRLKSEDTAMYYCTSPPLGGAWFGYWGQGTLVTVS

SEQ ID NO: 49 - protein, 118 aa, Mus musculus
EVNLVESGGGLVQPGGSLKLSCEASGFSFSDYYMYWVRQTPEKRLEWVTYITNGGGRTYYSDTIRGRFTISRDNARNTLYLQMSRLKSEDTAMYYCTSPPLGGAWFGYWGQGTLVTVS

SEQ ID NO: 50 - protein, 118 aa, Mus musculus
EVNLVESGGGLVQPGGSLKLSCAASGFTFSDYYLYWVRQTPEKRLEWVTYITNGGGRTYYSDTVRGRFTISRDNAKNTLYLQMSRLKSEDTAMYYCASPPLGGAWFGYWGQGTLVTVS

SEQ ID NO: 51 - protein, 118 aa, Mus musculus
EVNLVESGGGLVQPGGSLKLSCAASGFTFSDYYMYWVRQTPEKRLEWVTYITNGGGRTYYSDTVRGRFTISRDNAKNTLYLQMSRLKSEDTAMYYCASPPLGGAWFGYWGQGTLVTVS

SEQ ID NO: 52 - protein, 118 aa, Mus musculus
EVNLVESGGGLVQPGGSLKLSCEASGFTFSDYYMYWVRQTPEKRLEWVTYITNGAGRTYYSDTVRGRFTISRDNAKNTLYLQMSRLKSEDTAMYYCASPPLGGAWFAYWGQGTLVTVS

SEQ ID NO: 53 - protein, 118 aa, Mus musculus
EVKLVESGGGLVQPGGSLKLSCAASGFPFSDYYLYWVRQTPEKRLEWVAYITNGVGRTYYSDSVKGRFTISRDNAKNTLYLQMSRLKSEDTSMYYCTSPPLGGAWFAYWGQGTLVTVS

SEQ ID NO: 54 - protein, 117 aa, Mus musculus
QDQLQQSGVALARPGASVKLSCKASEYTFKSFGLGWVKQRPGQGLEWIGEIYPRSGNTYYNEKVKGKVRLTADKSSNSVYMEFRSLTSEDSAVYFCARSPYDGYFDYWGQGTTLTVS

SEQ ID NO: 55 - protein, 113 aa, Mus musculus
DVLMTQTPLSLPVSLGDQASISCRSSQSIVHSNGNTYLEWYLQKPGQSPKLLIYKVSKRFSGVPDRFSGSGSGTDFTLKISRVEAEDLGVYYCFQGSHVPLTFGAGTKLELKR

SEQ ID NO: 56 - protein, 113 aa, Mus musculus
DVSMTQTPLSLPVSLGDQASISCRSSQSLVHSNGNTYLEWYLQKPGQSPKLLIYKVSKRFSGVPDRFSGSGSGTDFTLKISRVEAEDLGVYYCFQGSHVPLTFGAGTKLELKR

SEQ ID NO: 57 - protein, 113 aa, Mus musculus; identical to SEQ ID NO: 55

SEQ ID NO: 58 - protein, 113 aa, Mus musculus
DVLMTQTPLSLPVSLGDQASISCRSSQSLVHSNGNTYLEWYLQKPGQSPKLLIYKVSKRFSGVPDRFSGSGSGTDFTLKISRVGAEDLGVYYCFQGSHVPLTFGAGTKLELKR

SEQ ID NO: 59 - protein, 113 aa, Mus musculus
DVVMTHSPLSLPVSLGDQASISCRSSQSLLHINGNTYLHWYLQKPGQSPKLLIYRVSNRLSGVPDRFSGSGSGTDFTLKISRVEAEDLGVYFCSQSTHVPRTFGGGTKLEIKR

SEQ ID NO: 60 - protein, 330 aa, Mus musculus [sequence omitted]

SEQ ID NO: 61 - protein, 330 aa, Mus musculus
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 330 aa, Mus musculus
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - DNA, 354 bp, Mus musculus
gaagtgaagttggtggagtctgggggaggcttagtgcagcctggagggtccctgaaactctcctgtgcagcctctggattcccttttagtgactattacctgtattgggttcgccagactccagagaagaggctggagtgggtcgcatacatcactaatggtgttggtaggacctattattcagactctgtaaagggccgattcaccatctccagagacaatgccaagaacaccctgtacctgcaaatgagccgtctgaagtctgaggacacatccatgtattactgtactagtcccccactggggggggcctggtttgcttactggggccaagggactctggtcactgtctct

SEQ ID NO: 64 - DNA, 354 bp, Mus musculus
gaagtgaacctggtggagtctgggggaggcttagtgcagcctggagggtccctgaaactctcctgtgcagcctctggattcactttcagtgactattacatgtattgggttcgccagactccagagaagaggctggagtgggtcacatatattactaatggtgatggtaggacctattattcagacactgtaaggggccgattcaccatatccagagacaatgccaagaacaccctgtacctgcaaatgagccgtctgaagtctgaggacacagccatgtattactgtacaagtcctccactgggaggggcctggtttggttactggggccaagggactctggtcactgtctct

SEQ ID NO: 65 - DNA, 354 bp, Mus musculus
gaagtgaacctggtggagtctgggggaggcttagtgcagcctggagggtccctgaaactctcctgtgaagcctctggattcagtttcagtgactattacatgtattgggttcgccagactccagagaagcggctggagtgggtcacatatattactaatggtggtggtagaacctattactcagacactataaggggccgattcaccatctccagagacaatgccaggaacaccctgtacctgcaaatgagccgtctgaagtctgaggacacagccatgtattactgtacaagtcccccactggggggggcctggtttggttactggggccaagggactctggtcactgtttct

SEQ ID NO: 66 - DNA, 354 bp, Mus musculus
gaagtgaacctggtggagtctgggggaggcttagtgcagcctggagggtccctgaaactctcctgtgcagcctctggattcactttcagtgactattatttatattgggttcgccagactccagagaagaggctggagtgggtcacatatattactaatggtggtggtaggacctattattcagacactgtaaggggccgattcaccatatccagagacaatgccaagaacaccctgtacctgcaaatgagccgtctgaagtctgaggacacagccatgtattactgtgcaagtcctccactgggaggggcctggtttggttactggggccaagggactctggtcactgtctct

SEQ ID NO: 67 - DNA, 354 bp, Mus musculus
gaagtgaacctggtggagtctgggggaggcttagtgcagcctggagggtccctgaaactctcctgtgcagcctctggattcactttcagtgactattacatgtattgggttcgccagactccagagaagaggctggagtgggtcacatatattactaatggtggtggtaggacctattattcagacactgtaaggggccgattcaccatatccagagacaatgccaagaacaccctgtacctacaaatgagccgtctgaagtctgaggacacagccatgtattactgtgcaagtcctccactgggaggggcctggtttggttactggggccaagggactctggtcactgtctct

SEQ ID NO: 68 - DNA, 354 bp, Mus musculus
gaagtgaacctggtggagtctgggggaggcttagtgcagcctggagggtccctgaaactctcctgtgaagcctctggattcactttcagtgactattacatgtattgggttcgccagactccagagaagaggctggagtgggtcacatatattactaatggtgctggtagaacctattactcagacactgtaaggggccgattcaccatctccagagacaatgccaagaacaccctgtacctgcaaatgagccgtctgaagtctgaggacacagccatgtattactgtgcaagtcccccactgggaggggcctggtttgcttactggggccaagggactctggtcactgtctct

SEQ ID NO: 69 - DNA, 351 bp, Mus musculus
caggatcagttacagcagtctggagttgcgctggcgaggcctggggcttcagtgaagctgtcctgcaaggcttctgaatacaccttcaaaagctttggtttaggctgggtgaagcagagacctggtcagggccttgagtggattggagagatttatcctagaagtggtaatacttactacaatgagaaggtcaagggcaaggtcagactgactgcagacaaatcctccaactcagtttacatggagttccgcagcctgacatctgaggactctgcggtctatttctgtgcacgatccccctatgatggttactttgactactggggccaaggcaccactctcacagtctcc

SEQ ID NO: 70 - DNA, 339 bp, Mus musculus
gatgttttgatgacccaaactccactctccctgcctgtcagtcttggagatcaagcctccatctcttgcagatctagtcagagtattgtacatagtaatggaaacacctatttagaatggtacctgcagaaaccaggccagtctccaaagctcctgatctacaaagtttccaaacgattttctggggtcccagacaggttcagtggcagtggatcagggacagatttcacactcaagatcagcagagtggaggctgaagatctgggagtttattactgttttcaaggttcacatgttccgctcacgttcggtgctgggaccaagctggagctgaaacgg

SEQ ID NO: 71 - DNA, 339 bp, Mus musculus
gatgttttgatgacccaaactccactctccctgcctgtcagtcttggagatcaagcctccatctcttgcagatctagtcagagtattgtacatagtaatggaaacacctatttagaatggtacctgcagaaaccaggccagtctccaaagctcctgatctacaaagtttccaaacgattttctggggtcccagacaggttcagtggcagtggatcagggacagatttcacactcaagatcagcagagtggaggctgaggatctgggagtttattactgttttcaaggttcacatgttccgctcacgttcggtgctgggaccaaactggagctgaaacgg

SEQ ID NO: 72 - DNA, 339 bp, Mus musculus
gatgtttcgatgacccaaactccactctccctgcctgtcagtcttggagatcaagcctccatctcttgcagatctagtcagagccttgtacatagtaatggaaacacctatttagaatggtacctgcagaaaccaggccagtctccaaagctcctgatctacaaagtttccaagcgattttctggggtcccagacaggttcagtggcagtggatcagggacagatttcacactcaagatcagcagagtggaggctgaggatctgggagtttattactgttttcaaggttcacatgttccgctcacgttcggtgctgggaccaagctggagctgaaacgg

SEQ ID NO: 73 - DNA, 339 bp, Mus musculus; identical to SEQ ID NO: 70

SEQ ID NO: 74 - DNA, 339 bp, Mus musculus
gatgttttgatgacccaaactccactctccctgcctgtcagtcttggagatcaagcctccatctcttgcagatctagtcagagtcttgtacatagtaatggaaacacctatttagaatggtacctgcagaaaccaggccagtctccaaagctcctgatctacaaagtttccaaacgattttctggggtcccagacaggttcagtggcagtggatcagggacagatttcacactcaagatcagcagagtgggggctgaggatctgggagtttattactgttttcaaggttcacatgttccgctcacgttcggtgctgggaccaagctggagctgaaacgg

SEQ ID NO: 75 - DNA, 339 bp, Mus musculus
gatgtttcgatgacccaaactccactctccctgcctgtcagtcttggagatcaagcctccatctcttgcagatctagtcagagccttgtacatagcaatggaaacacctatttagaatggtacctgcagaaaccaggccagtctccaaagctcctgatctacaaagtttccaaacgattttctggggtcccagacaggttcagtggcagtggttcagggacagatttcacactcaagatcagcagagtggaggctgaggatctgggagtttattactgttttcaaggttcacatgttccgctcacgttcggtgctgggaccaagctggagctgaaacgg

SEQ ID NO: 76 - DNA, 339 bp, Mus musculus
gatgttgtgatgacccattctccactctccctacctgtcagtcttggagatcaggcctccatctcttgcagatctagtcagagccttctgcacattaatggaaacacctatttacattggtacctgcagaagccaggccagtctccaaagctcctgatctacagagtttccaaccgattatctggggtcccagacaggttcagtggcagtggatcagggacagatttcacactcaagatcagcagagtggaggctgaggatctgggagtttatttctgctctcaaagtacacatgttcctcggacgttcggtggaggcaccaagctggaaatcaaacgg

SEQ ID NO: 77 - DNA, 990 bp, Homo sapiens
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaattcgaggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagccagcatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagtctctccctgtctccgggtaaa

SEQ ID NO: 78 - DNA, 990 bp, Homo sapiens
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccggatgtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccattgcccgaggagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtggggagagcaatgggcagccggagaacaactacaagaccacgcctcccgtgttggactccgacggctccttcttcctctacagcaagctcaccgtggacaagagcagtggcagcaggggaacgtcttctcatgctccgtgatgcatgaggctctgcacaaccactacacgcagaagagtctctccctgtctccgggtaaa

SEQ ID NO: 79 - DNA, 990 bp, Homo sapiens
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagtctctccctgtctccgggtaaa

SEQ ID NO: 80 - protein, 25 aa, Homo sapiens [sequence omitted]

SEQ ID NO: 81 - protein, 17 aa, Homo sapiens
LYWVRQAPGKGLEWVAY

SEQ ID NO: 82 - protein, 38 aa, Homo sapiens
YYSDSVKGRFTISRDNAKNTLYLQMNSLRDEDTAVYYC

SEQ ID NO: 83 - protein, 10 aa, Homo sapiens
WGQGTLVTVS

SEQ ID NO: 84 - protein, 26 aa, Homo sapiens
DIVMTQSPLSLPVTPGEPASISCRSS

SEQ ID NO: 85 - protein, 17 aa, Homo sapiens
LEWYLQKPGQSPRLLIY

SEQ ID NO: 86 - protein, 36 aa, Homo sapiens
KRFSGVPDRFSGSGSGTDFTLKISRVEAEDVGVYYC

SEQ ID NO: 87 - protein, 11 aa, Homo sapiens
FGQGTKLEIKR

SEQ ID NO: 88 - protein, 118 aa, Homo sapiens
EVQLVESGGGLVQPGGSLRLSCAASGFPFSDYYLYWVRQAPGKGLEWVAYITNGVGRTYYSDSVKGRFTISRDNAKNTLYLQMNSLRDEDTAVYYCTSPPLGGAWFAYWGQGTLVTVS

SEQ ID NO: 89 - protein, 113 aa, Homo sapiens
DIVMTQSPLSLPVTPGEPASISCRSSQSIVHSNGNTYLEWYLQKPGQSPRLLIYKVSKRFSGVPDRFSGSGSGTDFTLKISRVEAEDVGVYYCFQGSHVPLTFGQGTKLEIKR

SEQ ID NO: 90 - protein, 25 aa, Mus musculus
EVNLVESGGGLVQPGGSLILSCEAS

SEQ ID NO: 91 - protein, 38 aa, Mus musculus
YYSDAIRGRFTISRDNARNTLYLQMSRLKSEDTAMYYC

SEQ ID NO: 92 - protein, 118 aa, Mus musculus
EVNLVESGGGLVQPGGSLILSCEASGFSFSDYYMYWVRQTPEKRLEWVTYITNGGGRTYYSDAIRGRFTISRDNARNTLYLQMSRLKSEDTAMYYCTSPPLGGAWFGYWGQGTLVTVS

SEQ ID NO: 93 - protein, 113 aa, Mus musculus; identical to SEQ ID NO: 56

SEQ ID NO: 94 - protein, 11 aa, Mus musculus
RSIVHSNGNTY

SEQ ID NO: 95 - protein, 25 aa, Mus musculus
EVQLVESGGGLVQPGGSLRLSCVAS

SEQ ID NO: 96 - protein, 118 aa, Mus musculus
EVQLVESGGGLVQPGGSLRLSCAASGFPFSDYYLYWVRQAPGKGLEWVAYITNGVGRTYYSDSVKGRFTISRDNAKNTLYLQMNSLRDEDTAVYYCTSPPLGGAWFGYWGQGTLVTVS

SEQ ID NO: 97 - protein, 118 aa, Mus musculus
EVQLVESGGGLVQPGGSLRLSCVASGFPFSDYYLYWVRQAPGKGLEWVAYITNGVGRTYYSDSVKGRFTISRDNAKNTLYLQMNSLRDEDTAVYYCTSPPLGGAWFGYWGQGTLVTVS

SEQ ID NO: 98 - protein, 113 aa, Mus musculus
DIVMTQSPLSLPVTPGEPASISCRSSRSIVHSNGNTYLEWYLQKPGQSPRLLIYKVSKRFSGVPDRFSGSGSGTDFTLKISRVEAEDVGVYYCFQGSHVPLTFGQGTKLEIKR

SEQ ID NO: 99 - DNA, 354 bp, Homo sapiens
gaggtgcagctggtggaatccggcggaggactggtgcagcctggcggctccctgagactgtcttgcgccgcctccggcttccccttctccgactactacctgtactgggtccgacaggccccaggcaagggcctggaatgggtggcctacatcaccaacggcgtgggccggacctactactccgactccgtgaagggccggttcaccatctcccgggacaacgccaagaacaccctgtacctgcagatgaactccctgcgggacgaggacaccgccgtgtactactgcacctccccacccctgggcggagcttggtttgcttactggggccagggcaccctggtcaccgtgtcc

SEQ ID NO: 100 - DNA, 336 bp, Homo sapiens
gacatcgtgatgacccagtcccccctgtccctgcctgtgacacctggcgagcccgcctccatctcctgccggtcctcccagtccatcgtgcactccaacggcaacacctacctggaatggtatctgcagaagcccggccagtcccctcggctgctgatctacaaggtgtccaagcggttctccggcgtgcccgacagattctccggctctggctccggcaccgacttcaccctgaagatctcccgggtggaagccgaggacgtgggcgtgtactactgttttcaaggctcccacgtgcccctgaccttcggccagggcaccaagctggaaatcaag

SEQ ID NO: 101 - protein, 29 aa, Homo sapiens
YFASVNTSYYTVDSEMLLCTLHEVRQFSR

SEQ ID NO: 102 - protein, 8 aa, Mus musculus
  MISC_FEATURE (1)..(1): X is equal to G or E
  MISC_FEATURE (2)..(2): X is equal to F or Y
  MISC_FEATURE (3)..(3): X is equal to T or S or P
  MISC_FEATURE (5)..(5): X is equal to S or K
  MISC_FEATURE (6)..(6): X is equal to D or S
  MISC_FEATURE (7)..(7): X is equal to Y or F
  MISC_FEATURE (8)..(8): X is equal to Y or G
XXXFXXXX

SEQ ID NO: 103 - protein, 8 aa, Mus musculus
  MISC_FEATURE (3)..(3): X is equal to S or T or P
GFXFSDYY

SEQ ID NO: 104 - protein, 8 aa, Mus musculus
  MISC_FEATURE (2)..(2): X is equal to T or Y
  MISC_FEATURE (3)..(3): X is equal to N or P
  MISC_FEATURE (4)..(4): X is equal to G or R
  MISC_FEATURE (5)..(5): X is equal to D or G or A or V or S
  MISC_FEATURE (7)..(7): X is equal to R or N
IXXXXGXT

SEQ ID NO: 105 - protein, 8 aa, Mus musculus
  MISC_FEATURE (5)..(5): X is equal to D or G or A or V
ITNGXGRT

SEQ ID NO: 106 - protein, 12 aa, Mus musculus
  MISC_FEATURE (1)..(1): X is equal to T or A
  MISC_FEATURE (2)..(2): X is equal to S or R
  MISC_FEATURE (3)..(3): X is equal to P or S
  MISC_FEATURE (5)..(5): X is equal to L or Y
  MISC_FEATURE (6)..(6): X is equal to G or D
  MISC_FEATURE (8)..(8): X is equal to A or no residue
  MISC_FEATURE (9)..(9): X is equal to W or Y
  MISC_FEATURE (11)..(11): X is equal to G or A or D
XXXPXXGXXFXY

SEQ ID NO: 107 - protein, 12 aa, Mus musculus
  MISC_FEATURE (1)..(1): X is equal to A or T
  MISC_FEATURE (11)..(11): X is equal to G or A
XSPPLGGAWFXY

SEQ ID NO: 108 - protein, 11 aa, Mus musculus
  MISC_FEATURE (3)..(3): X is equal to I or L
  MISC_FEATURE (4)..(4): X is equal to V or L
  MISC_FEATURE (6)..(6): X is equal to S or I
QSXXHXNGNTY

SEQ ID NO: 109 - protein, 11 aa, Mus musculus
  MISC_FEATURE (3)..(3): X is equal to I or L
QSXVHSNGNTY

SEQ ID NO: 110 - protein, 3 aa, Mus musculus
  MISC_FEATURE (1)..(1): X is equal to K or R
XVS

SEQ ID NO: 111 - protein, 9 aa, Mus musculus
  MISC_FEATURE (1)..(1): X is equal to F or S
  MISC_FEATURE (3)..(3): X is equal to G or S
  MISC_FEATURE (4)..(4): X is equal to S or T
  MISC_FEATURE (8)..(8): X is equal to L or R
XQXXHVPXT

(19) United States
(12) Patent Application Publication
(10) Pub. No.: US 2024/0109971 A1
(43) Pub. Date: Apr. 4, 2024

(54) CCR6 ANTIBODIES

(71) Applicant: MONASH UNIVERSITY, Clayton, Victoria (AU)

(72) Inventors: Remy ROBERT, Victoria (AU); Charles Reay MACKAY, Victoria (AU)

(21) Appl. No.: 18/266,445

(22) PCT Filed: Dec. 14, 2021
(86) PCT No.: PCT/AU21/51488

§ 371 Date: Jun. 9, 2023

(30) Foreign Application Priority Data
Dec. 14, 2020 (AU) 2020904653

(51) Int. Cl. C07K 16/28; A61P 17/06; A61P 37/06; C07K 14/715

(52) U.S. Cl. CPC C07K 16/2866; A61P 17/06; A61P 37/06; C07K 14/7158; A61K 2039/505; C07K 2317/569

(57) ABSTRACT

The invention relates to CCR6, to antibodies and related fragments thereof for binding to said receptors, to production of said antibodies and fragments and to use of said antibodies and fragments for detection and therapy of various conditions, in particular autoimmune diseases, inflammation, infection, oncology and fibrosis.

### FIELD OF THE INVENTION

The invention relates to CCR6, to antibodies and related fragments thereof for binding to said receptors, to production of said antibodies and fragments and to use of said antibodies and fragments for detection and therapy of various conditions, in particular inflammation, autoimmunity, infection and oncology.

### RELATED APPLICATION

This application claims priority from Australian provisional application AU 2020904653, the contents of which are hereby incorporated by reference in their entirety.

### BACKGROUND OF THE INVENTION

Chemokines are extracellular signalling molecules having diverse functions. They can initiate and/or maintain numerous cell processes, including chemotaxis, cell growth and in some cases, tumour growth, homing of malignant cells and metastasis.

Chemokines are also intimately involved in trafficking cells of immune system and are implicated in numerous autoimmune diseases, inflammation, and response to viral, bacterial and other infections. Chemokines can act by binding to, activating, or inhibiting receptors known as chemokine receptors—a class of G-protein coupled receptors (GPCRs) that are multispanning membrane proteins, in which the protein has one or more regions that span a cellular membrane.

The chemokine receptor 6 (CCR6; CD196) is expressed in immature dendritic cells, B-cell subsets (mature, naive and memory) and T-cells subsets (skin and gut homing effector/memory T cells and Th17 cells). It is involved in the migration of Th17 cells to inflamed tissues and is implicated in lymphocyte activation and trafficking. The chemotaxis of cells induced through binding of the CCR6 receptor by its major ligand MIP-3α (CCL20 CKb4; LARC; MIP-3a; MIP3A, SCYA20, ST38), plays an important role in homeostatic and inflammatory processes in mucosal surfaces, skin, brain, and eye.

In inflammation and particularly autoimmune diseases, autocrine and paracrine mechanisms based in part on signalling via the CCR6-MIP-3α axes can be involved.

There is a need for improved reagents for binding to CCR6, particularly for new antibodies and fragments thereof that are capable of binding CCR6 and inhibiting MIP-3α mediated activity, for the detection and therapy of various conditions, in particular inflammation, infection, oncology and fibrosis.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention provides an antigen binding protein for treating a disease associated with CCR6 expression. Preferably the antigen binding protein inhibits binding of MIP-3α to CCR6.

The present invention provides an antigen binding protein for binding to a peptide, wherein the peptide:
consists of the sequence of SEQ ID NO: 2; or
consists of a sequence within the sequence of SEQ ID NO: 2, said peptide being useful as an immunogen to generate an antibody that is capable of binding to a CCR6.

The present invention provides a peptide, wherein the peptide:
consists of the sequence of SEQ ID NO: 2;
consists of a sequence within the sequence of SEQ ID NO: 2, said peptide being useful as an immunogen to generate an antibody that is capable of binding to a CCR6.

The present invention also provides an antigen binding protein that binds to:
a peptide consisting of amino acids 1 to 28 of CCR6, and
a peptide consisting of amino acids 18 to 46 of CCR6. Preferably, the CCR6 is human.

The invention provides an antigen binding protein for binding to CCR6, the antigen binding protein comprising:

FR1-CDR1-FR2-CDR2-FR3-CDR3-FR4, and

FR1a-CDR1a-FR2a-CDR2a-FR3a-CDR3a-FR4a,
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the sequence of any of the framework regions or complementarity determining regions are as described herein.

The invention provides an antigen binding protein for binding to CCR6, the antigen binding protein including:

FR1-CDR1-FR2-CDR2-FR3-CDR3-FR4, and

FR1a-CDR1a-FR2a-CDR2a-FR3a-CDR3a-FR4a,
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the sequence of any of the complementarity determining regions have an amino acid sequence as described in Table 1 or 2 below. Preferably, the framework regions have an amino acid sequence also as described in Table 3 or 4 below, including amino acid variation at particular residues which can be determined by aligning the various framework regions derived from each antibody. The invention also includes where CDR1, CDR2 and CDR3 are sequences from the variable heavy chain of an antibody (a VH), CDR1a, CDR2a and CDR3a are sequences from the variable light chain of an antibody (a VL), or where CDR1, CDR2 and CDR3 are sequences from the VL, CDR1a, CDR2a and CDR3a are sequences from VH.

In any embodiment, the invention provides an antigen binding protein for binding to CCR6, wherein the antigen binding protein comprises a CDR1 of a variable heavy chain, the CDR1 comprising the amino acid sequence as set forth in any one of SEQ ID NOs: 3, 6, 11, 14 102 or 103, preferably wherein the sequence of the CDR1 of the variable heavy chain comprises the amino acid sequence as set forth in SEQ ID NO: 11.

In any embodiment, the invention provides an antigen binding protein for binding to CCR6, wherein the antigen binding protein comprises a CDR2 of a variable heavy chain, the CDR2 comprising the amino acid sequence as set forth in any one of SEQ ID NOs: 4, 7, 9, 12, 15, or 104, preferably wherein the sequence of the CDR2 of the variable heavy chain comprises the amino acid sequence as set forth in SEQ ID NO: 12.

In any embodiment, the invention provides an antigen binding protein for binding to CCR6, wherein the antigen binding protein comprises a CDR3 of a variable heavy chain, the CDR3 comprising the amino acid sequence as set forth in any one of SEQ ID NOs: 5, 8, 10, 13, 16, 106 or 107, preferably wherein the sequence of the CDR3 of the variable heavy variable chain comprises the amino acid sequence as set forth in SEQ ID NO: 5.

In any embodiment, the invention provides an antigen binding protein for binding to CCR6, wherein the antigen binding protein comprises a CDR1 of a variable light chain, the CDR1 comprising the amino acid sequence as set forth in any one of SEQ ID NOs: 17, 20, 21, 94, 108 or 109, preferably wherein the sequence of the CDR1 of the variable light chain comprises the amino acid sequence as set forth in SEQ ID NO: 17 or 94.

In any embodiment, the invention provides an antigen binding protein for binding to CCR6, wherein the antigen binding protein comprises a CDR2 of a variable light chain, the CDR2 comprising the amino acid sequence as set forth in any one of SEQ ID NOs:18, 22 or 110, preferably wherein the sequence of the CDR2 of the variable light chain comprises the amino acid sequence as set forth in SEQ ID NO: 18.

In any embodiment, the invention provides an antigen binding protein for binding to CCR6, wherein the antigen binding protein comprises a CDR3 of a variable light chain, the CDR3 comprising the amino acid sequence as set forth in any one of SEQ ID NOs: 19, 23 or 111, preferably wherein the sequence of the CDR3 of the variable light chain comprises the amino acid sequence as set forth in SEQ ID NO: 19.

In any embodiment, the invention provides an antigen binding protein for binding to CCR6, wherein the antigen binding protein comprises a variable heavy chain comprising CDRs 1, 2 and 3, wherein CDR1 comprises the amino acid sequence SEQ ID NO: 11, CDR2 comprises the amino acid sequence as set forth in SEQ ID NO: 12 and CDR3 comprises the amino acid sequence as set forth in SEQ ID NO: 5.

In any embodiment, the invention provides an antigen binding protein for binding to CCR6, wherein the antigen binding protein comprises a variable light chain comprising CDRs 1, 2 and 3, wherein CDR1 comprises the amino acid sequence SEQ ID NO: 17 or 94, CDR2 comprises the amino acid sequence as set forth in SEQ ID NO: 18 and CDR3 comprises the amino acid sequence as set forth in SEQ ID NO: 19.

In any embodiment, the invention provides an antigen binding protein for binding to CCR6, wherein the antigen binding protein competitively inhibits the binding to CCR6 of an antibody:
comprising a VH comprising a sequence as set forth in SEQ ID NO: 88 and a VL comprising a sequence as set forth in SEQ ID NO: 89;
comprising a VH comprising a sequence as set forth in SEQ ID NO: 96 and a VL comprising a sequence as set forth in SEQ ID NO: 98;
comprising a VH comprising a sequence as set forth in SEQ ID NO: 97 and a VL comprising a sequence as set forth in SEQ ID NO: 98; or
comprising a VH comprising a sequence as set forth in SEQ ID NO: 88 and a VL comprising a sequence as set forth in SEQ ID NO: 98.

In any embodiment, the invention provides an antigen binding protein with a CDRH1, a CDRH2 and/or a CDRH3 of an antibody having a variable heavy chain as defined in any one of SEQ ID NOs: 88, 96, or 97.

In any embodiment, the invention provides an antigen binding protein with a CDRL1, a CDRL2 and/or a CDRL3 of an antibody having a variable light chain as defined in any one of SEQ ID NOs: 89 or 98.

In any embodiment, the invention provides an antigen binding protein with a CDR1, a CDR2 and/or a CDR3 of an antibody having a variable heavy chain as defined in any one of SEQ ID NOs: 88, 96, or 97 and a variable light chain as defined in any one of SEQ ID NOs: 89 or 98.

In any embodiment, an antigen binding protein described herein comprises:

FR1-CDR1-FR2-CDR2-FR3-CDR3-FR4-linker-FR1a-CDR1a-FR2a-CDR2a-FR3a-CDR3a-FR4a.

As defined herein, the linker may be a chemical, one or more amino acids, or a disulphide bond formed between two cysteine residues.

The present invention provides an antigen binding protein for binding to a CCR6 receptor, the antigen binding protein comprising:

FR1-CDR1-FR2-CDR2-FR3-CDR3-FR4, and

FR1a-CDR1a-FR2a-CDR2a-FR3a-CDR3a-FR4a,
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein:
CDR1 has a sequence selected from the group consisting of:

(SEQ ID NO: 102)
(G/E)(F/Y)(T/S/P)F(S/K)(D/S)(Y/F)(Y/G),
(SEQ ID NO: 103)
GF(S/T/P)FSDYY,
(SEQ ID NO: 3)
GFTFSDYY,
(SEQ ID NO: 6)
GFSFSDYY,
(SEQ ID NO: 11)
GFPFSDYY
and
(SEQ ID NO: 14)
EYTFKSFG;

CDR2 has a sequence selected from the group consisting of:

(SEQ ID NO: 104)
I(T/Y)(N/P)(G/R)(D/G/A/V/S)G(R/N)T,
(SEQ ID NO: 105)
ITNG(D/G/A/V)GRT,
(SEQ ID NO: 4)
ITNGDGRT,
(SEQ ID NO: 7)
ITNGGGRT,
(SEQ ID NO: 9)
ITNGAGRT,
(SEQ ID NO: 12)
ITNGVGRT
and
(SEQ ID NO: 15)
IYPRSGNT;

CDR3 has a sequence selected from the group consisting of:

(SEQ ID NO: 106)
(T/A)(S/R)(P/S)P(L/Y)(G/D)G(A/-)(W/Y)F(G/A/D)Y,
(SEQ ID NO: 107)
(A/T)SPPLGGAWF(G/A)Y,
(SEQ ID NO: 5)
TSPPLGGAWFGY,
(SEQ ID NO: 8)
ASPPLGGAWFGY,
(SEQ ID NO: 10)
ASPPLGGAWFAY,
(SEQ ID NO: 13)
TSPPLGGAWFAY
and
(SEQ ID NO: 16)
ARSPYDGYFDY;

CDR1a has a sequence selected from the group consisting of:

(SEQ ID NO: 108)
QS(I/L)(V/L)H(S/I)NGNTY,
(SEQ ID NO: 109)
QS(I/L)VHSNGNTY,
(SEQ ID NO: 17)
QSIVHSNGNTY,
(SEQ ID NO: 20)
QSLVHSNGNTY
and
(SEQ ID NO: 21)
QSLLHINGNTY;

CDR2a has a sequence selected from the group consisting of: (K/R)VS (SEQ ID NO: 110), RVS (SEQ ID NO: 22) and KVS (SEQ ID NO: 18); and
CDR3a has a sequence selected from the group consisting of:

(SEQ ID NO: 111)
(F/S)Q(G/S)(S/T)HVP(L/R)T,
(SEQ ID NO: 19)
FQGSHVPLT
and
(SEQ ID NO: 23)
SQSTHVPRT;

wherein preferably:
FR1 has a sequence selected from the groups consisting of:

(SEQ ID NO: 24)
EVNLVESGGGLVQPGGSLKLSCAAS,
(SEQ ID NO: 25)
EVNLVESGGGLVQPGGSLKLSCEAS,
(SEQ ID NO: 26)
EVKLVESGGGLVQPGGSLKLSCAAS,
(SEQ ID NO: 27)
QDQLQQSGVALARPGASVKLSCKAS,
(SEQ ID NO: 90)
EVNLVESGGGLVQPGGSLILSCEAS
and
(SEQ ID NO: 80)
EVQLVESGGGLVQPGGSLRLSCAAS;

FR2 has a sequence selected from the group consisting of:

(SEQ ID NO: 28)
MYWVRQTPEKRLEWVTY,
(SEQ ID NO: 29)
LYWVRQTPEKRLEWVTY,
(SEQ ID NO: 30)
LYWVRQTPEKRLEWVAY,
(SEQ ID NO: 31)
LGWVKQRPGQGLEWIGE
and
(SEQ ID NO: 81)
LYWVRQAPGKGLEWVAY;

FR3 has a sequence selected from the group consisting of:

(SEQ ID NO: 32)
YYSDTVRGRFTISRDNAKNTLYLQMSRLKSEDTAMYYC,
(SEQ ID NO: 33)
YYSDTIRGRFTISRDNARNTLYLQMSRLKSEDTAMYYC,
(SEQ ID NO: 34)
YYSDSVKGRFTISRDNAKNTLYLQMSRLKSEDTSMYYC,
(SEQ ID NO: 35)
YYNEKVKGKVRLTADKSSNSVYMEFRSLTSEDSAVYFC,
(SEQ ID NO: 91)
YYSDAIRGRFTISRDNARNTLYLQMSRLKSEDTAMYYC
and
(SEQ ID NO: 82)
YYSDSVKGRFTISRDNAKNTLYLQMNSLRDEDTAVYYC;

FR4 has a sequence: WGQGTLVTVS (SEQ ID NO: 36) or WGQGTTLTVS (SEQ ID NO: 37);
FR1a has a sequence selected from the group consisting of:

(SEQ ID NO: 38)
DVLMTQTPLSLPVSLGDQASISCRSS,
(SEQ ID NO: 39)
DVSMTQTPLSLPVSLGDQASISCRSS,
(SEQ ID NO: 40)
DVVMTHSPLSLPVSLGDQASISCRSS
and
(SEQ ID NO: 84)
DIVMTQSPLSLPVTPGEPASISCRSS;

FR2a has a sequence: LEWYLQKPGQSPKLLIY (SEQ ID NO: 41), LHWYLQKPGQSPKLLIY (SEQ ID NO: 42) or LEWYLQKPGQSPRLLIY (SEQ ID NO: 85);
FR3a has a sequence selected from the group consisting of:

(SEQ ID NO: 43)
KRFSGVPDRFSGSGSGTDFTLKISRVEAEDLGVYYC,
(SEQ ID NO: 44)
KRFSGVPDRFSGSGSGTDFTLKISRVGAEDLGVYYC,
(SEQ ID NO: 45)
NRLSGVPDRFSGSGSGTDFTLKISRVEAEDLGVYFC
and
(SEQ ID NO: 86)
KRFSGVPDRFSGSGSGTDFTLKISRVEAEDVGVYYC;
and

FR4a has a sequence: FGAGTKLELKR (SEQ ID NO: 46), FGGGTKLEIKR (SEQ ID NO: 47) or FGQGTKLEIR (SEQ ID NO: 87).

The invention provides an antigen binding protein including, consisting essentially of or consisting of an amino acids sequence of any one of SEQ ID NOs: 48 to 59, 88, 89, 92, 93, and 96 to 98.

The present invention also provides an antigen binding protein comprising an antigen binding domain of an antibody, wherein the antigen binding domain binds to or specifically binds to CCR6, wherein the antigen binding domain comprises at least one of:
(i) a VH comprising a complementarity determining region (CDR) 1 comprising a sequence at least about 60%, at least 70%, at least 75%, at least 80%, at least 85%, at least 90%, at least 92%, at least 95%, at least 97%, at least 99% identical to a sequence set forth in SEQ ID NO:3, a CDR2 comprising a sequence at least about 60%, at least 70%, at least 75%, at least 80%, at least 85%, at least 90%, at least 92%, at least 95%, at least 97%, at least 99% identical to a sequence set in SEQ ID NO:4 and a CDR3 comprising a sequence at least about 60%, at least 70%, at least 75%, at least 80%, at least 85%, at least 90%, at least 92%, at least 95%, at least 97%, at least 99% identical to a sequence set forth in SEQ ID NO: 5;
(ii) a VH comprising a sequence at least about 80%, at least 85%, at least 90%, at least 92%, at least 95% at least 96%, at least 97%, at least 98%, at least 99% identical to a sequence set forth in SEQ ID NO: 48;
(iii) a VL comprising a CDR1 comprising a sequence at least about 60%, at least 70%, at least 75%, at least 80%, at least 85%, at least 90%, at least 92%, at least 95%, at least 97%, at least 99% identical to a sequence set forth in SEQ ID NO: 17, a CDR2 comprising a sequence at least about 65%, at least about 66%, at least 80%, at least 85%, at least 90%, at least 92%, at least 95%, at least 97%, at least 99% identical to a sequence set forth in SEQ ID NO: 18 and a CDR3 comprising a sequence at least about 60%, at least about 70%, at least about 75%, at least 80%, at least 85%, at least 90%, at least 92%, at least 95%, at least 97%, at least 99% identical to a sequence set forth in SEQ ID NO: 19;
(iv) a VL comprising a sequence at least about at least about 80%, at least 85%, at least 90%, at least 92%, at least 95% at least 96%, at least 97%, at least 98%, at least 99% identical to a sequence set forth in SEQ ID NO: 55;
(v) a VH comprising a CDR1 comprising a sequence set forth in SEQ ID NO: 3, a CDR2 comprising a sequence set forth in SEQ ID NO: 4 and a CDR3 comprising a sequence set forth in SEQ ID NO: 5;
(vi) a VH comprising a sequence set forth in SEQ ID NO: 48;
(vii) a VL comprising a CDR1 comprising a sequence set SEQ ID NO: 17, a CDR2 comprising a sequence set forth in SEQ ID NO: 18 and a CDR3 comprising a sequence set forth in SEQ ID NO: 19;
(viii) a VL comprising a sequence set forth in SEQ ID NO: 55;
(ix) a VH comprising a CDR1 comprising a sequence set forth in SEQ ID NO: 3, a CDR2 comprising a sequence set forth in SEQ ID NO: 4 and a CDR3 comprising a sequence set forth in SEQ ID NO: 5; and a VL comprising a CDR1 comprising a sequence set SEQ ID NO: 17, a CDR2 comprising a sequence set forth in SEQ ID NO: 18 and a CDR3 comprising a sequence set forth in SEQ ID NO: 19; or
(x) a VH comprising a sequence set forth in SEQ ID NO: 48 and a VL comprising a sequence set forth in SEQ ID NO: 55.

In any aspect of the invention, the antigen binding domain further comprises at least one of:
(i) a VH comprising a framework region (FR) 1 comprising a sequence at least about 80%, at least 85%, at least 90%, at least 92%, at least 95%, at least 97%, at least 99% identical to a sequence set forth in SEQ ID NO:24, a FR2 comprising a sequence at least about 80%, at least 85%, at least 90%, at least 92%, at least 95%, at least 97%, at least 99% identical to a sequence set in SEQ ID NO:28, a FR3 comprising a sequence at least about 80%, at least 85%, at least 90%, at least 92%, at least 95%, at least 97%, at least 99% identical to a sequence set forth in SEQ ID NO: 32, and a FR4 comprising a sequence at least about 80%, at least 85%, at least 90%, at least 92%, at least 95%, at least 97%, at least 99% identical to a sequence set forth in SEQ ID NO: 36;
(ii) a VL comprising a FR1 comprising a sequence at least about 80%, at least 85%, at least 90%, at least 92%, at least 95%, at least 97%, at least 99% identical to a sequence set forth in SEQ ID NO: 38, a FR2 comprising a sequence at least about 80%, at least 85%, at least 90%, at least 92%, at least 95%, at least 97%, at least 99% identical to a sequence set forth in SEQ ID NO: 41, a FR3 comprising a sequence at least about 80%, at least 85%, at least 90%, at least 92%, at least 95%, at least 97%, at least 99% identical to a sequence set forth in SEQ ID NO: 43, and a FR4 comprising a sequence at least about 80%, at least 85%, at least 90%, at least 92%, at least 95%, at least 97%, at least 99% identical to a sequence set forth in SEQ ID NO: 46;
(iii) a VH comprising a FR1 comprising a sequence set forth in SEQ ID NO: 24, a FR2 comprising a sequence set forth in SEQ ID NO: 28, a FR3 comprising a sequence set forth in SEQ ID NO: 32, and a FR4 comprising a sequence set forth in SEQ ID NO: 36;
(iv) a VL comprising a FR1 comprising a sequence set forth in SEQ ID NO: 38, a FR2 comprising a sequence set forth in SEQ ID NO: 41, a FR3 comprising a sequence set forth in SEQ ID NO: 43, and a FR4 comprising a sequence set forth in SEQ ID NO: 46; or
(v) a VH comprising a FR1 comprising a sequence set forth in SEQ ID NO: 24, a FR2 comprising a sequence set forth in SEQ ID NO: 28, a FR3 comprising a sequence set forth in SEQ ID NO: 32, and a FR4 comprising a sequence set forth in SEQ ID NO: 36; and a VL comprising a FR1 comprising a sequence set forth in SEQ ID NO: 38, a FR2 comprising a sequence set forth in SEQ ID NO: 41, a FR3 comprising a sequence set forth in SEQ ID NO: 43, and a FR4 comprising a sequence set forth in SEQ ID NO: 46.

The present invention also provides an antigen binding protein comprising an antigen binding domain of an antibody, wherein the antigen binding domain binds to or specifically binds to CCR6, wherein the antigen binding domain comprises at least one of:
(i) a VH comprising a complementarity determining region (CDR) 1 comprising a sequence at least about 60%, at least 70%, at least 75%, at least 80%, at least 85%, at least 90%, at least 92%, at least 95%, at least 97%, at least 99% identical to a sequence set forth in SEQ ID NO:6, a CDR2 comprising a sequence at least about 60%, at least 70%, at least 75%, at least 80%, at least 85%, at least 90%, at least 92%, at least 95%, at least 97%, at least 99% identical to a sequence set in SEQ ID NO:7 and a CDR3 comprising a sequence at least about 60%, at least 70%, at least 75%, at least 80%, at least 85%, at least 90%, at least 92%, at least 95%, at least 97%, at least 99% identical to a sequence set forth in SEQ ID NO: 5;
(ii) a VH comprising a sequence at least about 80%, at least 85%, at least 90%, at least 92%, at least 95% at least 96%, at least 97%, at least 98%, at least 99% identical to a sequence set forth in SEQ ID NO: 49;
(iii) a VL comprising a CDR1 comprising a sequence at least about 60%, at least 70%, at least 75%, at least 80%, at least 85%, at least 90%, at least 92%, at least 95%, at least 97%, at least 99% identical to a sequence set forth in SEQ ID NO: 20, a CDR2 comprising a sequence at least about 65%, at least about 66%, at least 80%, at least 85%, at least 90%, at least 92%, at least 95%, at least 97%, at least 99% identical to a sequence set forth in SEQ ID NO: 18 and a CDR3 comprising a sequence at least about 60%, at least about 70%, at least about 75%, at least 80%, at least 85%, at least 90%, at least 92%, at least 95%, at least 97%, at least 99% identical to a sequence set forth in SEQ ID NO: 19;
(iv) a VL comprising a sequence at least about at least about 80%, at least 85%, at least 90%, at least 92%, at least 95% at least 96%, at least 97%, at least 98%, at least 99% identical to a sequence set forth in SEQ ID NO: 56;
(v) a VH comprising a CDR1 comprising a sequence set forth in SEQ ID NO: 6, a CDR2 comprising a sequence set forth in SEQ ID NO: 7 and a CDR3 comprising a sequence set forth in SEQ ID NO: 5;
(vi) a VH comprising a sequence set forth in SEQ ID NO: 49;
(vii) a VL comprising a CDR1 comprising a sequence set SEQ ID NO: 20, a CDR2 comprising a sequence set forth in SEQ ID NO: 18 and a CDR3 comprising a sequence set forth in SEQ ID NO: 19;
(viii) a VL comprising a sequence set forth in SEQ ID NO: 56;
(ix) a VH comprising a CDR1 comprising a sequence set forth in SEQ ID NO: 6, a CDR2 comprising a sequence set forth in SEQ ID NO: 7 and a CDR3 comprising a sequence set forth in SEQ ID NO: 5; and a VL comprising a CDR1 comprising a sequence set SEQ ID NO: 20, a CDR2 comprising a sequence set forth in SEQ ID NO: 18 and a CDR3 comprising a sequence set forth in SEQ ID NO: 19; or
(x) a VH comprising a sequence set forth in SEQ ID NO: 49 and a VL comprising a sequence set forth in SEQ ID NO: 56.

In any aspect of the invention, the antigen binding domain further comprises at least one of:
(i) a VH comprising a framework region (FR) 1 comprising a sequence at least about 80%, at least 85%, at least 90%, at least 92%, at least 95%, at least 97%, at least 99% identical to a sequence set forth in SEQ ID NO:25, a FR2 comprising a sequence at least about 80%, at least 85%, at least 90%, at least 92%, at least 95%, at least 97%, at least 99% identical to a sequence set in SEQ ID NO:28, a FR3 comprising a sequence at least about 80%, at least 85%, at least 90%, at least 92%, at least 95%, at least 97%, at least 99% identical to a sequence set forth in SEQ ID NO: 33, and a FR4 comprising a sequence at least about 80%, at least 85%, at least 90%, at least 92%, at least 95%, at least 97%, at least 99% identical to a sequence set forth in SEQ ID NO: 36;
(ii) a VL comprising a FR1 comprising a sequence at least about 80%, at least 85%, at least 90%, at least 92%, at least 95%, at least 97%, at least 99% identical to a sequence set forth in SEQ ID NO: 39, a FR2 comprising a sequence at least about 80%, at least 85%, at least 90%, at least 92%, at least 95%, at least 97%, at least 99% identical to a sequence set forth in SEQ ID NO: 41, a FR3 comprising a sequence at least about 80%, at least 85%, at least 90%, at least 92%, at least 95%, at least 97%, at least 99% identical to a sequence set forth in SEQ ID NO: 43, and a FR4 comprising a sequence at least about 80%, at least 85%, at least 90%, at least 92%, at least 95%, at least 97%, at least 99% identical to a sequence set forth in SEQ ID NO: 46;
(iii) a VH comprising a FR1 comprising a sequence set forth in SEQ ID NO: 25, a FR2 comprising a sequence set forth in SEQ ID NO: 28, a FR3 comprising a sequence set forth in SEQ ID NO: 33, and a FR4 comprising a sequence set forth in SEQ ID NO: 36;
(iv) a VL comprising a FR1 comprising a sequence set forth in SEQ ID NO: 39, a FR2 comprising a sequence set forth in SEQ ID NO: 41, a FR3 comprising a sequence set forth in SEQ ID NO: 43, and a FR4 comprising a sequence set forth in SEQ ID NO: 46; or
(v) a VH comprising a FR1 comprising a sequence set forth in SEQ ID NO: 25, a FR2 comprising a sequence set forth in SEQ ID NO: 28, a FR3 comprising a sequence set forth in SEQ ID NO: 33, and a FR4 comprising a sequence set forth in SEQ ID NO: 36; and a VL comprising a FR1 comprising a sequence set forth in SEQ ID NO: 39, a FR2 comprising a sequence set forth in SEQ ID NO: 41, a FR3 comprising a sequence set forth in SEQ ID NO: 43, and a FR4 comprising a sequence set forth in SEQ ID NO: 46.

The present invention also provides an antigen binding protein comprising an antigen binding domain of an antibody, wherein the antigen binding domain binds to or specifically binds to CCR6, wherein the antigen binding domain comprises at least one of:
(i) a VH comprising a complementarity determining region (CDR) 1 comprising a sequence at least about 60%, at least 70%, at least 75%, at least 80%, at least 85%, at least 90%, at least 92%, at least 95%, at least 97%, at least 99% identical to a sequence set forth in SEQ ID NO:3, a CDR2 comprising a sequence at least about 60%, at least 70%, at least 75%, at least 80%, at least 85%, at least 90%, at least 92%, at least 95%, at least 97%, at least 99% identical to a sequence set in SEQ ID NO:7 and a CDR3 comprising a sequence at least about 60%, at least 70%, at least 75%, at least 80%, at least 85%, at least 90%, at least 92%, at least 95%, at least 97%, at least 99% identical to a sequence set forth in SEQ ID NO: 8;
(ii) a VH comprising a sequence at least about 80%, at least 85%, at least 90%, at least 92%, at least 95% at least 96%, at least 97%, at least 98%, at least 99% identical to a sequence set forth in SEQ ID NO: 50;
(iii) a VL comprising a CDR1 comprising a sequence at least about 60%, at least 70%, at least 75%, at least 80%, at least 85%, at least 90%, at least 92%, at least 95%, at least 97%, at least 99% identical to a sequence set forth in SEQ ID NO: 17, a CDR2 comprising a sequence at least about 65%, at least about 66%, at least 80%, at least 85%, at least 90%, at least 92%, at least 95%, at least 97%, at least 99% identical to a sequence set forth in SEQ ID NO: 18 and a CDR3 comprising a sequence at least about 60%, at least about 70%, at least about 75%, at least 80%, at least 85%, at least 90%, at least 92%, at least 95%, at least 97%, at least 99% identical to a sequence set forth in SEQ ID NO: 19;
(iv) a VL comprising a sequence at least about at least about 80%, at least 85%, at least 90%, at least 92%, at least 95% at least 96%, at least 97%, at least 98%, at least 99% identical to a sequence set forth in SEQ ID NO: 57;
(v) a VH comprising a CDR1 comprising a sequence set forth in SEQ ID NO: 3, a CDR2 comprising a sequence set forth in SEQ ID NO: 7 and a CDR3 comprising a sequence set forth in SEQ ID NO: 8;
(vi) a VH comprising a sequence set forth in SEQ ID NO: 50;
(vii) a VL comprising a CDR1 comprising a sequence set SEQ ID NO: 17, a CDR2 comprising a sequence set forth in SEQ ID NO: 18 and a CDR3 comprising a sequence set forth in SEQ ID NO: 19;
(viii) a VL comprising a sequence set forth in SEQ ID NO: 57;
(ix) a VH comprising a CDR1 comprising a sequence set forth in SEQ ID NO: 3, a CDR2 comprising a sequence set forth in SEQ ID NO: 7 and a CDR3 comprising a sequence set forth in SEQ ID NO: 8; and a VL comprising a CDR1 comprising a sequence set SEQ ID NO: 17, a CDR2 comprising a sequence set forth in SEQ ID NO: 18 and a CDR3 comprising a sequence set forth in SEQ ID NO: 19; or
(x) a VH comprising a sequence set forth in SEQ ID NO: 50 and a VL comprising a sequence set forth in SEQ ID NO: 57.

In any aspect of the invention, the antigen binding domain further comprises at least one of:
(i) a VH comprising a framework region (FR) 1 comprising a sequence at least about 80%, at least 85%, at least 90%, at least 92%, at least 95%, at least 97%, at least 99% identical to a sequence set forth in SEQ ID NO:24, a FR2 comprising a sequence at least about 80%, at least 85%, at least 90%, at least 92%, at least 95%, at least 97%, at least 99% identical to a sequence set in SEQ ID NO:29, a FR3 comprising a sequence at least about 80%, at least 85%, at least 90%, at least 92%, at least 95%, at least 97%, at least 99% identical to a sequence set forth in SEQ ID NO: 32, and a FR4 comprising a sequence at least about 80%, at least 85%, at least 90%, at least 92%, at least 95%, at least 97%, at least 99% identical to a sequence set forth in SEQ ID NO: 36;
(ii) a VL comprising a FR1 comprising a sequence at least about 80%, at least 85%, at least 90%, at least 92%, at least 95%, at least 97%, at least 99% identical to a sequence set forth in SEQ ID NO: 38, a FR2 comprising a sequence at least about 80%, at least 85%, at least 90%, at least 92%, at least 95%, at least 97%, at least 99% identical to a sequence set forth in SEQ ID NO: 41, a FR3 comprising a sequence at least about 80%, at least 85%, at least 90%, at least 92%, at least 95%, at least 97%, at least 99% identical to a sequence set forth in SEQ ID NO: 43, and a FR4 comprising a sequence at least about 80%, at least 85%, at least 90%, at least 92%, at least 95%, at least 97%, at least 99% identical to a sequence set forth in SEQ ID NO: 46;
(iii) a VH comprising a FR1 comprising a sequence set forth in SEQ ID NO: 24, a FR2 comprising a sequence set forth in SEQ ID NO: 28, a FR3 comprising a sequence set forth in SEQ ID NO: 32, and a FR4 comprising a sequence set forth in SEQ ID NO: 36;
(iv) a VL comprising a FR1 comprising a sequence set forth in SEQ ID NO: 38, a FR2 comprising a sequence set forth in SEQ ID NO: 41, a FR3 comprising a sequence set forth in SEQ ID NO: 43, and a FR4 comprising a sequence set forth in SEQ ID NO: 46; or
(v) a VH comprising a FR1 comprising a sequence set forth in SEQ ID NO: 24, a FR2 comprising a sequence set forth in SEQ ID NO: 28, a FR3 comprising a sequence set forth in SEQ ID NO: 32, and a FR4 comprising a sequence set forth in SEQ ID NO: 36; and a VL comprising a FR1 comprising a sequence set forth in SEQ ID NO: 38, a FR2 comprising a sequence set forth in SEQ ID NO: 41, a FR3 comprising a sequence set forth in SEQ ID NO: 43, and a FR4 comprising a sequence set forth in SEQ ID NO: 46.

The present invention also provides an antigen binding protein comprising an antigen binding domain of an antibody, wherein the antigen binding domain binds to or specifically binds to CCR6, wherein the antigen binding domain comprises at least one of:
(i) a VH comprising a complementarity determining region (CDR) 1 comprising a sequence at least about 60%, at least 70%, at least 75%, at least 80%, at least 85%, at least 90%, at least 92%, at least 95%, at least 97%, at least 99% identical to a sequence set forth in SEQ ID NO:3, a CDR2 comprising a sequence at least about 60%, at least 70%, at least 75%, at least 80%, at least 85%, at least 90%, at least 92%, at least 95%, at least 97%, at least 99% identical to a sequence set in SEQ ID NO:7 and a CDR3 comprising a sequence at least about 60%, at least 70%, at least 75%, at least 80%, at least 85%, at least 90%, at least 92%, at least 95%, at least 97%, at least 99% identical to a sequence set forth in SEQ ID NO: 8;
(ii) a VH comprising a sequence at least about 80%, at least 85%, at least 90%, at least 92%, at least 95% at least 96%, at least 97%, at least 98%, at least 99% identical to a sequence set forth in SEQ ID NO: 51;
(iii) a VL comprising a CDR1 comprising a sequence at least about 60%, at least 70%, at least 75%, at least 80%, at least 85%, at least 90%, at least 92%, at least 95%, at least 97%, at least 99% identical to a sequence set forth in SEQ ID NO: 20, a CDR2 comprising a sequence at least about 65%, at least about 66%, at least 80%, at least 85%, at least 90%, at least 92%, at least 95%, at least 97%, at least 99% identical to a sequence set forth in SEQ ID NO: 18 and a CDR3 comprising a sequence at least about 60%, at least about 70%, at least about 75%, at least 80%, at least 85%, at least 90%, at least 92%, at least 95%, at least 97%, at least 99% identical to a sequence set forth in SEQ ID NO: 19;
(iv) a VL comprising a sequence at least about at least about 80%, at least 85%, at least 90%, at least 92%, at least 95% at least 96%, at least 97%, at least 98%, at least 99% identical to a sequence set forth in SEQ ID NO: 58;
(v) a VH comprising a CDR1 comprising a sequence set forth in SEQ ID NO: 3, a CDR2 comprising a sequence set forth in SEQ ID NO: 7 and a CDR3 comprising a sequence set forth in SEQ ID NO: 8;
(vi) a VH comprising a sequence set forth in SEQ ID NO: 51;
(vii) a VL comprising a CDR1 comprising a sequence set SEQ ID NO: 20, a CDR2 comprising a sequence set forth in SEQ ID NO: 18 and a CDR3 comprising a sequence set forth in SEQ ID NO: 19;
(viii) a VL comprising a sequence set forth in SEQ ID NO: 58;
(ix) a VH comprising a CDR1 comprising a sequence set forth in SEQ ID NO: 3, a CDR2 comprising a sequence set forth in SEQ ID NO: 7 and a CDR3 comprising a sequence set forth in SEQ ID NO: 8; and a VL comprising a CDR1 comprising a sequence set SEQ ID NO: 20, a CDR2 comprising a sequence set forth in SEQ ID NO: 18 and a CDR3 comprising a sequence set forth in SEQ ID NO: 19; or
(x) a VH comprising a sequence set forth in SEQ ID NO: 51 and a VL comprising a sequence set forth in SEQ ID NO: 58.

In any aspect of the invention, the antigen binding domain further comprises at least one of:
(i) a VH comprising a framework region (FR) 1 comprising a sequence at least about 80%, at least 85%, at least 90%, at least 92%, at least 95%, at least 97%, at least 99% identical to a sequence set forth in SEQ ID NO:24, a FR2 comprising a sequence at least about 80%, at least 85%, at least 90%, at least 92%, at least 95%, at least 97%, at least 99% identical to a sequence set in SEQ ID NO:28, a FR3 comprising a sequence at least about 80%, at least 85%, at least 90%, at least 92%, at least 95%, at least 97%, at least 99% identical to a sequence set forth in SEQ ID NO: 32, and a FR4 comprising a sequence at least about 80%, at least 85%, at least 90%, at least 92%, at least 95%, at least 97%, at least 99% identical to a sequence set forth in SEQ ID NO: 36;
(ii) a VL comprising a FR1 comprising a sequence at least about 80%, at least 85%, at least 90%, at least 92%, at least 95%, at least 97%, at least 99% identical to a sequence set forth in SEQ ID NO: 38, a FR2 comprising a sequence at least about 80%, at least 85%, at least 90%, at least 92%, at least 95%, at least 97%, at least 99% identical to a sequence set forth in SEQ ID NO: 41, a FR3 comprising a sequence at least about 80%, at least 85%, at least 90%, at least 92%, at least 95%, at least 97%, at least 99% identical to a sequence set forth in SEQ ID NO: 44, and a FR4 comprising a sequence at least about 80%, at least 85%, at least 90%, at least 92%, at least 95%, at least 97%, at least 99% identical to a sequence set forth in SEQ ID NO: 46;
(iii) a VH comprising a FR1 comprising a sequence set forth in SEQ ID NO: 24, a FR2 comprising a sequence set forth in SEQ ID NO: 28, a FR3 comprising a sequence set forth in SEQ ID NO: 32, and a FR4 comprising a sequence set forth in SEQ ID NO: 36;
(iv) a VL comprising a FR1 comprising a sequence set forth in SEQ ID NO: 38, a FR2 comprising a sequence set forth in SEQ ID NO: 41, a FR3 comprising a sequence set forth in SEQ ID NO: 44, and a FR4 comprising a sequence set forth in SEQ ID NO: 46; or
(v) a VH comprising a FR1 comprising a sequence set forth in SEQ ID NO: 24, a FR2 comprising a sequence set forth in SEQ ID NO: 28, a FR3 comprising a sequence set forth in SEQ ID NO: 32, and a FR4 comprising a sequence set forth in SEQ ID NO: 36; and a VL comprising a FR1 comprising a sequence set forth in SEQ ID NO: 38, a FR2 comprising a sequence set forth in SEQ ID NO: 41, a FR3 comprising a sequence set forth in SEQ ID NO: 44, and a FR4 comprising a sequence set forth in SEQ ID NO: 46.

The present invention also provides an antigen binding protein comprising an antigen binding domain of an antibody, wherein the antigen binding domain binds to or specifically binds to CCR6, wherein the antigen binding domain comprises at least one of:
(i) a VH comprising a complementarity determining region (CDR) 1 comprising a sequence at least about 60%, at least 70%, at least 75%, at least 80%, at least 85%, at least 90%, at least 92%, at least 95%, at least 97%, at least 99% identical to a sequence set forth in SEQ ID NO:3, a CDR2 comprising a sequence at least about 60%, at least 70%, at least 75%, at least 80%, at least 85%, at least 90%, at least 92%, at least 95%, at least 97%, at least 99% identical to a sequence set in SEQ ID NO:9 and a CDR3 comprising a sequence at least about 60%, at least 70%, at least 75%, at least 80%, at least 85%, at least 90%, at least 92%, at least 95%, at least 97%, at least 99% identical to a sequence set forth in SEQ ID NO: 10;
(ii) a VH comprising a sequence at least about 80%, at least 85%, at least 90%, at least 92%, at least 95% at least 96%, at least 97%, at least 98%, at least 99% identical to a sequence set forth in SEQ ID NO: 52;
(iii) a VL comprising a CDR1 comprising a sequence at least about 60%, at least 70%, at least 75%, at least 80%, at least 85%, at least 90%, at least 92%, at least 95%, at least 97%, at least 99% identical to a sequence set forth in SEQ ID NO: 20, a CDR2 comprising a sequence at least about 65%, at least about 66%, at least 80%, at least 85%, at least 90%, at least 92%, at least 95%, at least 97%, at least 99% identical to a sequence set forth in SEQ ID NO: 18 and a CDR3 comprising a sequence at least about 60%, at least about 70%, at least about 75%, at least 80%, at least 85%, at least 90%, at least 92%, at least 95%, at least 97%, at least 99% identical to a sequence set forth in SEQ ID NO: 19;
(iv) a VL comprising a sequence at least about at least about 80%, at least 85%, at least 90%, at least 92%, at least 95% at least 96%, at least 97%, at least 98%, at least 99% identical to a sequence set forth in SEQ ID NO: 56;
(v) a VH comprising a CDR1 comprising a sequence set forth in SEQ ID NO: 3, a CDR2 comprising a sequence set forth in SEQ ID NO: 9 and a CDR3 comprising a sequence set forth in SEQ ID NO: 10;
(vi) a VH comprising a sequence set forth in SEQ ID NO: 52;
(vii) a VL comprising a CDR1 comprising a sequence set SEQ ID NO: 20, a CDR2 comprising a sequence set forth in SEQ ID NO: 18 and a CDR3 comprising a sequence set forth in SEQ ID NO: 19;
(viii) a VL comprising a sequence set forth in SEQ ID NO: 56;
(ix) a VH comprising a CDR1 comprising a sequence set forth in SEQ ID NO: 3, a CDR2 comprising a sequence set forth in SEQ ID NO: 9 and a CDR3 comprising a sequence set forth in SEQ ID NO: 10; and a VL comprising a CDR1 comprising a sequence set SEQ ID NO: 20, a CDR2 comprising a sequence set forth in SEQ ID NO: 18 and a CDR3 comprising a sequence set forth in SEQ ID NO: 19; or
(x) a VH comprising a sequence set forth in SEQ ID NO: 52 and a VL comprising a sequence set forth in SEQ ID NO: 56.

In any aspect of the invention, the antigen binding domain further comprises at least one of:
(i) a VH comprising a framework region (FR) 1 comprising a sequence at least about 80%, at least 85%, at least 90%, at least 92%, at least 95%, at least 97%, at least 99% identical to a sequence set forth in SEQ ID NO:25, a FR2 comprising a sequence at least about 80%, at least 85%, at least 90%, at least 92%, at least 95%, at least 97%, at least 99% identical to a sequence set in SEQ ID NO:28, a FR3 comprising a sequence at least about 80%, at least 85%, at least 90%, at least 92%, at least 95%, at least 97%, at least 99% identical to a sequence set forth in SEQ ID NO: 32, and a FR4 comprising a sequence at least about 80%, at least 85%, at least 90%, at least 92%, at least 95%, at least 97%, at least 99% identical to a sequence set forth in SEQ ID NO: 36;
(ii) a VL comprising a FR1 comprising a sequence at least about 80%, at least 85%, at least 90%, at least 92%, at least 95%, at least 97%, at least 99% identical to a sequence set forth in SEQ ID NO: 39, a FR2 comprising a sequence at least about 80%, at least 85%, at least 90%, at least 92%, at least 95%, at least 97%, at least 99% identical to a sequence set forth in SEQ ID NO: 41, a FR3 comprising a sequence at least about 80%, at least 85%, at least 90%, at least 92%, at least 95%, at least 97%, at least 99% identical to a sequence set forth in SEQ ID NO: 43, and a FR4 comprising a sequence at least about 80%, at least 85%, at least 90%, at least 92%, at least 95%, at least 97%, at least 99% identical to a sequence set forth in SEQ ID NO: 46;
(iii) a VH comprising a FR1 comprising a sequence set forth in SEQ ID NO: 25, a FR2 comprising a sequence set forth in SEQ ID NO: 28, a FR3 comprising a sequence set forth in SEQ ID NO: 32, and a FR4 comprising a sequence set forth in SEQ ID NO: 36;
(iv) a VL comprising a FR1 comprising a sequence set forth in SEQ ID NO: 39, a FR2 comprising a sequence set forth in SEQ ID NO: 41, a FR3 comprising a sequence set forth in SEQ ID NO: 43, and a FR4 comprising a sequence set forth in SEQ ID NO: 46; or
(v) a VH comprising a FR1 comprising a sequence set forth in SEQ ID NO: 25, a set forth in SEQ ID NO: 32, and a FR4 comprising a sequence set forth in SEQ ID NO: 36; and a VL comprising a FR1 comprising a sequence set forth in SEQ ID NO: 39, a FR2 comprising a sequence set forth in SEQ ID NO: 41, a FR3 comprising a sequence set forth in SEQ ID NO: 43, and a FR4 comprising a sequence set forth in SEQ ID NO: 46.

The present invention also provides an antigen binding protein comprising an antigen binding domain of an antibody, wherein the antigen binding domain binds to or specifically binds to CCR6, wherein the antigen binding domain comprises at least one of:
(i) a VH comprising a complementarity determining region (CDR) 1 comprising a sequence at least about 60%, at least 70%, at least 75%, at least 80%, at least 85%, at least 90%, at least 92%, at least 95%, at least 97%, at least 99% identical to a sequence set forth in SEQ ID NO:11, a CDR2 comprising a sequence at least about 60%, at least 70%, at least 75%, at least 80%, at least 85%, at least 90%, at least 92%, at least 95%, at least 97%, at least 99% identical to a sequence set in SEQ ID NO:12 and a CDR3 comprising a sequence at least about 60%, at least 70%, at least 75%, at least 80%, at least 85%, at least 90%, at least 92%, at least 95%, at least 97%, at least 99% identical to a sequence set forth in SEQ ID NO: 13;
(ii) a VH comprising a sequence at least about 80%, at least 85%, at least 90%, at least 92%, at least 95% at least 96%, at least 97%, at least 98%, at least 99% identical to a sequence set forth in SEQ ID NO: 53;
(iii) a VL comprising a CDR1 comprising a sequence at least about 60%, at least 70%, at least 75%, at least 80%, at least 85%, at least 90%, at least 92%, at least 95%, at least 97%, at least 99% identical to a sequence set forth in SEQ ID NO: 17, a CDR2 comprising a sequence at least about 65%, at least about 66%, at least 80%, at least 85%, at least 90%, at least 92%, at least 95%, at least 97%, at least 99% identical to a sequence set forth in SEQ ID NO: 18 and a CDR3 comprising a sequence at least about 60%, at least about 70%, at least about 75%, at least 80%, at least 85%, at least 90%, at least 92%, at least 95%, at least 97%, at least 99% identical to a sequence set forth in SEQ ID NO: 19;
(iv) a VL comprising a sequence at least about at least about 80%, at least 85%, at least 90%, at least 92%, at least 95% at least 96%, at least 97%, at least 98%, at least 99% identical to a sequence set forth in SEQ ID NO: 55;
(v) a VH comprising a CDR1 comprising a sequence set forth in SEQ ID NO: 11, a CDR2 comprising a sequence set forth in SEQ ID NO: 12 and a CDR3 comprising a sequence set forth in SEQ ID NO: 13;
(vi) a VH comprising a sequence set forth in SEQ ID NO: 53;
(vii) a VL comprising a CDR1 comprising a sequence set SEQ ID NO: 17, a CDR2 comprising a sequence set forth in SEQ ID NO: 18 and a CDR3 comprising a sequence set forth in SEQ ID NO: 19;
(viii) a VL comprising a sequence set forth in SEQ ID NO: 55;
(ix) a VH comprising a CDR1 comprising a sequence set forth in SEQ ID NO: 11, a CDR2 comprising a sequence set forth in SEQ ID NO: 12 and a CDR3 comprising a sequence set forth in SEQ ID NO: 13; and a VL comprising a CDR1 comprising a sequence set SEQ ID NO: 17, a CDR2 comprising a sequence set forth in SEQ ID NO: 18 and a CDR3 comprising a sequence set forth in SEQ ID NO: 19; or
(x) a VH comprising a sequence set forth in SEQ ID NO: 53 and a VL comprising a sequence set forth in SEQ ID NO: 55.

In any aspect of the invention, the antigen binding domain further comprises at least one of:
(i) a VH comprising a framework region (FR) 1 comprising a sequence at least about 80%, at least 85%, at least 90%, at least 92%, at least 95%, at least 97%, at least 99% identical to a sequence set forth in SEQ ID NO:26, a FR2 comprising a sequence at least about 80%, at least 85%, at least 90%, at least 92%, at least 95%, at least 97%, at least 99% identical to a sequence set in SEQ ID NO:30, a FR3 comprising a sequence at least about 80%, at least 85%, at least 90%, at least 92%, at least 95%, at least 97%, at least 99% identical to a sequence set forth in SEQ ID NO: 34, and a FR4 comprising a sequence at least about 80%, at least 85%, at least 90%, at least 92%, at least 95%, at least 97%, at least 99% identical to a sequence set forth in SEQ ID NO: 36;
(ii) a VL comprising a FR1 comprising a sequence at least about 80%, at least 85%, at least 90%, at least 92%, at least 95%, at least 97%, at least 99% identical to a sequence set forth in SEQ ID NO: 38, a FR2 comprising a sequence at least about 80%, at least 85%, at least 90%, at least 92%, at least 95%, at least 97%, at least 99% identical to a sequence set forth in SEQ ID NO: 41, a FR3 comprising a sequence at least about 80%, at least 85%, at least 90%, at least 92%, at least 95%, at least 97%, at least 99% identical to a sequence set forth in SEQ ID NO: 43, and a FR4 comprising a sequence at least about 80%, at least 85%, at least 90%, at least 92%, at least 95%, at least 97%, at least 99% identical to a sequence set forth in SEQ ID NO: 46;
(iii) a VH comprising a FR1 comprising a sequence set forth in SEQ ID NO: 26, a FR2 comprising a sequence set forth in SEQ ID NO: 30, a FR3 comprising a sequence set forth in SEQ ID NO: 34, and a FR4 comprising a sequence set forth in SEQ ID NO: 36;
(iv) a VL comprising a FR1 comprising a sequence set forth in SEQ ID NO: 38, a FR2 comprising a sequence set forth in SEQ ID NO: 41, a FR3 comprising a sequence set forth in SEQ ID NO: 43, and a FR4 comprising a sequence set forth in SEQ ID NO: 46; or
(v) a VH comprising a FR1 comprising a sequence set forth in SEQ ID NO: 26, a FR2 comprising a sequence set forth in SEQ ID NO: 30, a FR3 comprising a sequence set forth in SEQ ID NO: 34, and a FR4 comprising a sequence set forth in SEQ ID NO: 36; and a VL comprising a FR1 comprising a sequence set forth in SEQ ID NO: 38, a FR2 comprising a sequence set forth in SEQ ID NO: 41, a FR3 comprising a sequence set forth in SEQ ID NO: 43, and a FR4 comprising a sequence set forth in SEQ ID NO: 46.

In any aspect of the invention, the antigen binding domain further comprises at least one of:
(i) a VH comprising a framework region (FR) 1 comprising a sequence at least about 80%, at least 85%, at least 90%, at least 92%, at least 95%, at least 97%, at least 99% identical to a sequence set forth in SEQ ID NO:80, a FR2 comprising a sequence at least about 80%, at least 85%, at least 90%, at least 92%, at least 95%, at least 97%, at least 99% identical to a sequence set in SEQ ID NO:81, a FR3 comprising a sequence at least about 80%, at least 85%, at least 90%, at least 92%, at least 95%, at least 97%, at least 99% identical to a sequence set forth in SEQ ID NO: 82, and a FR4 comprising a sequence at least about 80%, at least 85%, at least 90%, at least 92%, at least 95%, at least 97%, at least 99% identical to a sequence set forth in SEQ ID NO: 83;
(ii) a VL comprising a FR1 comprising a sequence at least about 80%, at least 85%, at least 90%, at least 92%, at least 95%, at least 97%, at least 99% identical to a sequence set forth in SEQ ID NO: 84, a FR2 comprising a sequence at least about 80%, at least 85%, at least 90%, at least 92%, at least 95%, at least 97%, at least 99% identical to a sequence set forth in SEQ ID NO: 85, a FR3 comprising a sequence at least about 80%, at least 85%, at least 90%, at least 92%, at least 95%, at least 97%, at least 99% identical to a sequence set forth in SEQ ID NO: 86, and a FR4 comprising a sequence at least about 80%, at least 85%, at least 90%, at least 92%, at least 95%, at least 97%, at least 99% identical to a sequence set forth in SEQ ID NO: 87;
(iii) a VH comprising a FR1 comprising a sequence set forth in SEQ ID NO: 80, a FR2 comprising a sequence set forth in SEQ ID NO: 81, a FR3 comprising a sequence set forth in SEQ ID NO: 82, and a FR4 comprising a sequence set forth in SEQ ID NO: 83;
(iv) a VL comprising a FR1 comprising a sequence set forth in SEQ ID NO: 84, a FR2 comprising a sequence set forth in SEQ ID NO: 85, a FR3 comprising a sequence set forth in SEQ ID NO: 86, and a FR4 comprising a sequence set forth in SEQ ID NO: 87; or
(v) a VH comprising a FR1 comprising a sequence set forth in SEQ ID NO: 80, a FR2 comprising a sequence set forth in SEQ ID NO: 81, a FR3 comprising a sequence set forth in SEQ ID NO: 82, and a FR4 comprising a sequence set forth in SEQ ID NO: 83; and a VL comprising a FR1 comprising a sequence set forth in SEQ ID NO: 84, a FR2 comprising a sequence set forth in SEQ ID NO: 85, a FR3 comprising a sequence set forth in SEQ ID NO: 86, and a FR4 comprising a sequence set forth in SEQ ID NO: 87.

The present invention also provides an antigen binding protein comprising an antigen binding domain of an antibody, wherein the antigen binding domain binds to or specifically binds to CCR6, wherein the antigen binding domain comprises at least one of:
(i) a VH comprising a complementarity determining region (CDR) 1 comprising a sequence at least about 60%, at least 70%, at least 75%, at least 80%, at least 85%, at least 90%, at least 92%, at least 95%, at least 97%, at least 99% identical to a sequence set forth in SEQ ID NO:14, a CDR2 comprising a sequence at least about 60%, at least 70%, at least 75%, at least 80%, at least 85%, at least 90%, at least 92%, at least 95%, at least 97%, at least 99% identical to a sequence set in SEQ ID NO:15 and a CDR3 comprising a sequence at least about 60%, at least 70%, at least 75%, at least 80%, at least 85%, at least 90%, at least 92%, at least 95%, at least 97%, at least 99% identical to a sequence set forth in SEQ ID NO: 16;
(ii) a VH comprising a sequence at least about 80%, at least 85%, at least 90%, at least 92%, at least 95% at least 96%, at least 97%, at least 98%, at least 99% identical to a sequence set forth in SEQ ID NO: 54;
(iii) a VL comprising a CDR1 comprising a sequence at least about 60%, at least 70%, at least 75%, at least 80%, at least 85%, at least 90%, at least 92%, at least 95%, at least 97%, at least 99% identical to a sequence set forth in SEQ ID NO: 21, a CDR2 comprising a sequence at least about 65%, at least about 66%, at least 80%, at least 85%, at least 90%, at least 92%, at least 95%, at least 97%, at least 99% identical to a sequence set forth in SEQ ID NO: 22 and a CDR3 comprising a sequence at least about 60%, at least about 70%, at least about 75%, at least 80%, at least 85%, at least 90%, at least 92%, at least 95%, at least 97%, at least 99% identical to a sequence set forth in SEQ ID NO: 23;
(iv) a VL comprising a sequence at least about at least about 80%, at least 85%, at least 90%, at least 92%, at least 95% at least 96%, at least 97%, at least 98%, at least 99% identical to a sequence set forth in SEQ ID NO: 59;
(v) a VH comprising a CDR1 comprising a sequence set forth in SEQ ID NO: 14, a CDR2 comprising a sequence set forth in SEQ ID NO: 15 and a CDR3 comprising a sequence set forth in SEQ ID NO: 16;
(vi) a VH comprising a sequence set forth in SEQ ID NO: 54;
(vii) a VL comprising a CDR1 comprising a sequence set SEQ ID NO: 21, a CDR2 comprising a sequence set forth in SEQ ID NO: 22 and a CDR3 comprising a sequence set forth in SEQ ID NO: 23;
(viii) a VL comprising a sequence set forth in SEQ ID NO: 59;
(ix) a VH comprising a CDR1 comprising a sequence set forth in SEQ ID NO: 14, a CDR2 comprising a sequence set forth in SEQ ID NO: 15 and a CDR3 comprising a sequence set forth in SEQ ID NO: 16; and a VL comprising a CDR1 comprising a sequence set SEQ ID NO: 21, a CDR2 comprising a sequence set forth in SEQ ID NO: 22 and a CDR3 comprising a sequence set forth in SEQ ID NO: 23; or
(x) a VH comprising a sequence set forth in SEQ ID NO: 54 and a VL comprising a sequence set forth in SEQ ID NO: 59.

In any aspect of the invention, the antigen binding domain further comprises at least one of:
(i) a VH comprising a framework region (FR) 1 comprising a sequence at least about 80%, at least 85%, at least 90%, at least 92%, at least 95%, at least 97%, at least 99% identical to a sequence set forth in SEQ ID NO:27, a FR2 comprising a sequence at least about 80%, at least 85%, at least 90%, at least 92%, at least 95%, at least 97%, at least 99% identical to a sequence set in SEQ ID NO:31, a FR3 comprising a sequence at least about 80%, at least 85%, at least 90%, at least 92%, at least 95%, at least 97%, at least 99% identical to a sequence set forth in SEQ ID NO: 35, and a FR4 comprising a sequence at least about 80%, at least 85%, at least 90%, at least 92%, at least 95%, at least 97%, at least 99% identical to a sequence set forth in SEQ ID NO: 37;
(ii) a VL comprising a FR1 comprising a sequence at least about 80%, at least 85%, at least 90%, at least 92%, at least 95%, at least 97%, at least 99% identical to a sequence set forth in SEQ ID NO: 40, a FR2 comprising a sequence at least about 80%, at least 85%, at least 90%, at least 92%, at least 95%, at least 97%, at least 99% identical to a sequence set forth in SEQ ID NO: 42, a FR3 comprising a sequence at least about 80%, at least 85%, at least 90%, at least 92%, at least 95%, at least 97%, at least 99% identical to a sequence set forth in SEQ ID NO: 45, and a FR4 comprising a sequence at least about 80%, at least 85%, at least 90%, at least 92%, at least 95%, at least 97%, at least 99% identical to a sequence set forth in SEQ ID NO: 47;
(iii) a VH comprising a FR1 comprising a sequence set forth in SEQ ID NO: 27, a FR2 comprising a sequence set forth in SEQ ID NO: 31, a FR3 comprising a sequence set forth in SEQ ID NO: 35, and a FR4 comprising a sequence set forth in SEQ ID NO: 37;
(iv) a VL comprising a FR1 comprising a sequence set forth in SEQ ID NO: 40, a FR2 comprising a sequence set forth in SEQ ID NO: 42, a FR3 comprising a sequence set forth in SEQ ID NO: 45, and a FR4 comprising a sequence set forth in SEQ ID NO: 47; or
(v) a VH comprising a FR1 comprising a sequence set forth in SEQ ID NO: 27, a FR2 comprising a sequence set forth in SEQ ID NO: 31, a FR3 comprising a sequence set forth in SEQ ID NO: 35, and a FR4 comprising a sequence set forth in SEQ ID NO: 37; and a VL comprising a FR1 comprising a sequence set forth in SEQ ID NO: 40, a FR2 comprising a sequence set forth in SEQ ID NO: 42, a FR3 comprising a sequence set forth in SEQ ID NO: 45, and a FR4 comprising a sequence set forth in SEQ ID NO: 47.

The present invention also provides an antigen binding protein comprising an antigen binding domain of an antibody, wherein the antigen binding domain binds to or specifically binds to CCR6, wherein the antigen binding domain comprises at least one of:
(i) a VH comprising a complementarity determining region (CDR) 1 comprising a sequence at least about 60%, at least 70%, at least 75%, at least 80%, at least 85%, at least 90%, at least 92%, at least 95%, at least 97%, at least 99% identical to a sequence set forth in SEQ ID NO: 6, a CDR2 comprising a sequence at least about 60%, at least 70%, at least 75%, at least 80%, at least 85%, at least 90%, at least 92%, at least 95%, at least 97%, at least 99% identical to a sequence set in SEQ ID NO: 7 and a CDR3 comprising a sequence at least about 60%, at least 70%, at least 75%, at least 80%, at least 85%, at least 90%, at least 92%, at least 95%, at least 97%, at least 99% identical to a sequence set forth in SEQ ID NO: 5;
(ii) a VH comprising a sequence at least about 80%, at least 85%, at least 90%, at least 92%, at least 95% at least 96%, at least 97%, at least 98%, at least 99% identical to a sequence set forth in SEQ ID NO: 92;
(iii) a VL comprising a CDR1 comprising a sequence at least about 60%, at least 70%, at least 75%, at least 80%, at least 85%, at least 90%, at least 92%, at least 95%, at least 97%, at least 99% identical to a sequence set forth in SEQ ID NO: 20, a CDR2 comprising a sequence at least about 65%, at least about 66%, at least 80%, at least 85%, at least 90%, at least 92%, at least 95%, at least 97%, at least 99% identical to a sequence set forth in SEQ ID NO: 18 and a CDR3 comprising a sequence at least about 60%, at least about 70%, at least about 75%, at least 80%, at least 85%, at least 90%, at least 92%, at least 95%, at least 97%, at least 99% identical to a sequence set forth in SEQ ID NO: 19;
(iv) a VL comprising a sequence at least about at least about 80%, at least 85%, at least 90%, at least 92%, at least 95% at least 96%, at least 97%, at least 98%, at least 99% identical to a sequence set forth in SEQ ID NO: 93;
(v) a VH comprising a CDR1 comprising a sequence set forth in SEQ ID NO: 6, a CDR2 comprising a sequence set forth in SEQ ID NO: 7 and a CDR3 comprising a sequence set forth in SEQ ID NO: 5;
(vi) a VH comprising a sequence set forth in SEQ ID NO: 92;
(vii) a VL comprising a CDR1 comprising a sequence set SEQ ID NO: 20, a CDR2 comprising a sequence set forth in SEQ ID NO: 18 and a CDR3 comprising a sequence set forth in SEQ ID NO: 19;
(viii) a VL comprising a sequence set forth in SEQ ID NO: 93;
(ix) a VH comprising a CDR1 comprising a sequence set forth in SEQ ID NO: 6, a CDR2 comprising a sequence set forth in SEQ ID NO: 7 and a CDR3 comprising a sequence set forth in SEQ ID NO: 5; and a VL comprising a CDR1 comprising a sequence set SEQ ID NO: 20, a CDR2 comprising a sequence set forth in SEQ ID NO: 18 and a CDR3 comprising a sequence set forth in SEQ ID NO: 19; or
(x) a VH comprising a sequence set forth in SEQ ID NO: 92 and a VL comprising a sequence set forth in SEQ ID NO: 93.

In any aspect of the invention, the antigen binding domain further comprises at least one of:
(i) a VH comprising a framework region (FR) 1 comprising a sequence at least about 80%, at least 85%, at least 90%, at least 92%, at least 95%, at least 97%, at least 99% identical to a sequence set forth in SEQ ID NO: 90, a FR2 comprising a sequence at least about 80%, at least 85%, at least 90%, at least 92%, at least 95%, at least 97%, at least 99% identical to a sequence set in SEQ ID NO:28, a FR3 comprising a sequence at least about 80%, at least 85%, at least 90%, at least 92%, at least 95%, at least 97%, at least 99% identical to a sequence set forth in SEQ ID NO: 91, and a FR4 comprising a sequence at least about 80%, at least 85%, at least 90%, at least 92%, at least 95%, at least 97%, at least 99% identical to a sequence set forth in SEQ ID NO: 36;
(ii) a VL comprising a FR1 comprising a sequence at least about 80%, at least 85%, at least 90%, at least 92%, at least 95%, at least 97%, at least 99% identical to a sequence set forth in SEQ ID NO: 39, a FR2 comprising a sequence at least about 80%, at least 85%, at least 90%, at least 92%, at least 95%, at least 97%, at least 99% identical to a sequence set forth in SEQ ID NO: 41, a FR3 comprising a sequence at least about 80%, at least 85%, at least 90%, at least 92%, at least 95%, at least 97%, at least 99% identical to a sequence set forth in SEQ ID NO: 43, and a FR4 comprising a sequence at least about 80%, at least 85%, at least 90%, at least 92%, at least 95%, at least 97%, at least 99% identical to a sequence set forth in SEQ ID NO: 46;
(iii) a VH comprising a FR1 comprising a sequence set forth in SEQ ID NO: 90, a FR2 comprising a sequence set forth in SEQ ID NO: 28, a FR3 comprising a sequence set forth in SEQ ID NO: 91, and a FR4 comprising a sequence set forth in SEQ ID NO: 36;
(iv) a VL comprising a FR1 comprising a sequence set forth in SEQ ID NO: 39, a FR2 comprising a sequence set forth in SEQ ID NO: 41, a FR3 comprising a sequence set forth in SEQ ID NO: 43, and a FR4 comprising a sequence set forth in SEQ ID NO: 46; or
(v) a VH comprising a FR1 comprising a sequence set forth in SEQ ID NO: 90, a set forth in SEQ ID NO: 91, and a FR4 comprising a sequence set forth in SEQ ID NO: 36; and a VL comprising a FR1 comprising a sequence set forth in SEQ ID NO: 39, a FR2 comprising a sequence set forth in SEQ ID NO: 41, a FR3 comprising a sequence set forth in SEQ ID NO: 43, and a FR4 comprising a sequence set forth in SEQ ID NO: 46.

The present invention also provides an antigen binding protein comprising an antigen binding domain of an antibody, wherein the antigen binding domain binds to or specifically binds to CCR6, wherein the antigen binding domain comprises at least one of:
(i) a VH comprising a complementarity determining region (CDR) 1 comprising a sequence at least about 60%, at least 70%, at least 75%, at least 80%, at least 85%, at least 90%, at least 92%, at least 95%, at least 97%, at least 99% identical to a sequence set forth in SEQ ID NO:11, a CDR2 comprising a sequence at least about 60%, at least 70%, at least 75%, at least 80%, at least 85%, at least 90%, at least 92%, at least 95%, at least 97%, at least 99% identical to a sequence set in SEQ ID NO:12 and a CDR3 comprising a sequence at least about 60%, at least 70%, at least 75%, at least 80%, at least 85%, at least 90%, at least 92%, at least 95%, at least 97%, at least 99% identical to a sequence set forth in SEQ ID NO: 5;
(ii) a VH comprising a sequence at least about 80%, at least 85%, at least 90%, at least 92%, at least 95% at least 96%, at least 97%, at least 98%, at least 99% identical to a sequence set forth in SEQ ID NO: 96 or 97;
(iii) a VL comprising a CDR1 comprising a sequence at least about 60%, at least 70%, at least 75%, at least 80%, at least 85%, at least 90%, at least 92%, at least 95%, at least 97%, at least 99% identical to a sequence set forth in SEQ ID NO: 17, a CDR2 comprising a sequence at least about 65%, at least about 66%, at least 80%, at least 85%, at least 90%, at least 92%, at least 95%, at least 97%, at least 99% identical to a sequence set forth in SEQ ID NO: 18 and a CDR3 comprising a sequence at least about 60%, at least about 70%, at least about 75%, at least 80%, at least 85%, at least 90%, at least 92%, at least 95%, at least 97%, at least 99% identical to a sequence set forth in SEQ ID NO: 19;
(iv) a VL comprising a sequence at least about at least about 80%, at least 85%, at least 90%, at least 92%, at least 95% at least 96%, at least 97%, at least 98%, at least 99% identical to a sequence set forth in SEQ ID NO: 89;
(v) a VH comprising a CDR1 comprising a sequence set forth in SEQ ID NO: 11, a CDR2 comprising a sequence set forth in SEQ ID NO: 12 and a CDR3 comprising a sequence set forth in SEQ ID NO: 5;
(vi) a VH comprising a sequence set forth in SEQ ID NO: 96 or 97;
(vii) a VL comprising a CDR1 comprising a sequence set SEQ ID NO: 17, a CDR2 comprising a sequence set forth in SEQ ID NO: 18 and a CDR3 comprising a sequence set forth in SEQ ID NO: 19;
(viii) a VL comprising a sequence set forth in SEQ ID NO: 89;
(ix) a VH comprising a CDR1 comprising a sequence set forth in SEQ ID NO: 11, a CDR2 comprising a sequence set forth in SEQ ID NO: 12 and a CDR3 comprising a sequence set forth in SEQ ID NO: 5; and a VL comprising a CDR1 comprising a sequence set SEQ ID NO: 17, a CDR2 comprising a sequence set forth in SEQ ID NO: 18 and a CDR3 comprising a sequence set forth in SEQ ID NO: 19; or
(x) a VH comprising a sequence set forth in SEQ ID NO: 96 or 97 and a VL comprising a sequence set forth in SEQ ID NO: 89.

In any aspect of the invention, the antigen binding domain further comprises at least one of:
(i) a VH comprising a framework region (FR) 1 comprising a sequence at least about 80%, at least 85%, at least 90%, at least 92%, at least 95%, at least 97%, at least 99% identical to a sequence set forth in SEQ ID NO:80 or 95, a FR2 comprising a sequence at least about 80%, at least 85%, at least 90%, at least 92%, at least 95%, at least 97%, at least 99% identical to a sequence set in SEQ ID NO:81, a FR3 comprising a sequence at least about 80%, at least 85%, at least 90%, at least 92%, at least 95%, at least 97%, at least 99% identical to a sequence set forth in SEQ ID NO: 34, and a FR4 comprising a sequence at least about 80%, at least 85%, at least 90%, at least 92%, at least 95%, at least 97%, at least 99% identical to a sequence set forth in SEQ ID NO: 83;
(ii) a VL comprising a FR1 comprising a sequence at least about 80%, at least 85%, at least 90%, at least 92%, at least 95%, at least 97%, at least 99% identical to a sequence set forth in SEQ ID NO: 84, a FR2 comprising a sequence at least about 80%, at least 85%, at least 90%, at least 92%, at least 95%, at least 97%, at least 99% identical to a sequence set forth in SEQ ID NO: 85, a FR3 comprising a sequence at least about 80%, at least 85%, at least 90%, at least 92%, at least 95%, at least 97%, at least 99% identical to a sequence set forth in SEQ ID NO: 86, and a FR4 comprising a sequence at least about 80%, at least 85%, at least 90%, at least 92%, at least 95%, at least 97%, at least 99% identical to a sequence set forth in SEQ ID NO: 87;
(iii) a VH comprising a FR1 comprising a sequence set forth in SEQ ID NO: 80 or 95, a FR2 comprising a sequence set forth in SEQ ID NO: 81, a FR3 comprising a sequence set forth in SEQ ID NO: 34, and a FR4 comprising a sequence set forth in SEQ ID NO: 83;
(iv) a VL comprising a FR1 comprising a sequence set forth in SEQ ID NO: 84, a FR2 comprising a sequence set forth in SEQ ID NO: 85, a FR3 comprising a sequence set forth in SEQ ID NO: 86, and a FR4 comprising a sequence set forth in SEQ ID NO: 87; or
(v) a VH comprising a FR1 comprising a sequence set forth in SEQ ID NO: 80 or 95, a FR2 comprising a sequence set forth in SEQ ID NO: 81, a FR3 comprising a sequence set forth in SEQ ID NO: 34, and a FR4 comprising a sequence set forth in SEQ ID NO: 83; and a VL comprising a FR1 comprising a sequence set forth in SEQ ID NO: 84, a FR2 comprising a sequence set forth in SEQ ID NO: 85, a FR3 comprising a sequence set forth in SEQ ID NO: 86, and a FR4 comprising a sequence set forth in SEQ ID NO: 87.

The present invention also provides an antigen binding protein comprising an antigen binding domain of an antibody, wherein the antigen binding domain binds to or specifically binds to CCR6, wherein the antigen binding domain comprises at least one of:
(i) a VH comprising a complementarity determining region (CDR) 1 comprising a sequence at least about 60%, at least 70%, at least 75%, at least 80%, at least 85%, at least 90%, at least 92%, at least 95%, at least 97%, at least 99% identical to a sequence set forth in SEQ ID NO:11, a CDR2 comprising a sequence at least about 60%, at least 70%, at least 75%, at least 80%, at least 85%, at least 90%, at least 92%, at least 95%, at least 97%, at least 99% identical to a sequence set in SEQ ID NO:12 and a CDR3 comprising a sequence at least about 60%, at least 70%, at least 75%, at least 80%, at least 85%, at least 90%, at least 92%, at least 95%, at least 97%, at least 99% identical to a sequence set forth in SEQ ID NO: 13;
(ii) a VH comprising a sequence at least about 80%, at least 85%, at least 90%, at least 92%, at least 95% at least 96%, at least 97%, at least 98%, at least 99% identical to a sequence set forth in SEQ ID NO: 88;
(iii) a VL comprising a CDR1 comprising a sequence at least about 60%, at least 70%, at least 75%, at least 80%, at least 85%, at least 90%, at least 92%, at least 95%, at least 97%, at least 99% identical to a sequence set forth in SEQ ID NO: 94, a CDR2 comprising a sequence at least about 65%, at least about 66%, at least 80%, at least 85%, at least 90%, at least 92%, at least 95%, at least 97%, at least 99% identical to a sequence set forth in SEQ ID NO: 18 and a CDR3 comprising a sequence at least about 60%, at least about 70%, at least about 75%, at least 80%, at least 85%, at least 90%, at least 92%, at least 95%, at least 97%, at least 99% identical to a sequence set forth in SEQ ID NO: 19;
(iv) a VL comprising a sequence at least about at least about 80%, at least 85%, at least 90%, at least 92%, at least 95% at least 96%, at least 97%, at least 98%, at least 99% identical to a sequence set forth in SEQ ID NO: 98;
(v) a VH comprising a CDR1 comprising a sequence set forth in SEQ ID NO: 11, a CDR2 comprising a sequence set forth in SEQ ID NO: 12 and a CDR3 comprising a sequence set forth in SEQ ID NO: 13;
(vi) a VH comprising a sequence set forth in SEQ ID NO: 88;
(vii) a VL comprising a CDR1 comprising a sequence set SEQ ID NO: 94, a CDR2 comprising a sequence set forth in SEQ ID NO: 18 and a CDR3 comprising a sequence set forth in SEQ ID NO: 19;
(viii) a VL comprising a sequence set forth in SEQ ID NO: 98;
(ix) a VH comprising a CDR1 comprising a sequence set forth in SEQ ID NO: 11, a CDR2 comprising a sequence set forth in SEQ ID NO: 12 and a CDR3 comprising a sequence set forth in SEQ ID NO: 13; and a VL comprising a CDR1 comprising a sequence set SEQ ID NO: 94, a CDR2 comprising a sequence set forth in SEQ ID NO: 18 and a CDR3 comprising a sequence set forth in SEQ ID NO: 19; or
(x) a VH comprising a sequence set forth in SEQ ID NO: 88 and a VL comprising a sequence set forth in SEQ ID NO: 98.

In any aspect of the invention, the antigen binding domain further comprises at least one of:
(i) a VH comprising a framework region (FR) 1 comprising a sequence at least about 80%, at least 85%, at least 90%, at least 92%, at least 95%, at least 97%, at least 99% identical to a sequence set forth in SEQ ID NO:80, a FR2 comprising a sequence at least about 80%, at least 85%, at least 90%, at least 92%, at least 95%, at least 97%, at least 99% identical to a sequence set in SEQ ID NO:30, a FR3 comprising a sequence at least about 80%, at least 85%, at least 90%, at least 92%, at least 95%, at least 97%, at least 99% identical to a sequence set forth in SEQ ID NO: 82, and a FR4 comprising a sequence at least about 80%, at least 85%, at least 90%, at least 92%, at least 95%, at least 97%, at least 99% identical to a sequence set forth in SEQ ID NO: 83;
(ii) a VL comprising a FR1 comprising a sequence at least about 80%, at least 85%, at least 90%, at least 92%, at least 95%, at least 97%, at least 99% identical to a sequence set forth in SEQ ID NO: 84, a FR2 comprising a sequence at least about 80%, at least 85%, at least 90%, at least 92%, at least 95%, at least 97%, at least 99% identical to a sequence set forth in SEQ ID NO: 85, a FR3 comprising a sequence at least about 80%, at least 85%, at least 90%, at least 92%, at least 95%, at least 97%, at least 99% identical to a sequence set forth in SEQ ID NO: 86, and a FR4 comprising a sequence at least about 80%, at least 85%, at least 90%, at least 92%, at least 95%, at least 97%, at least 99% identical to a sequence set forth in SEQ ID NO: 87;
(iii) a VH comprising a FR1 comprising a sequence set forth in SEQ ID NO: 80, a FR2 comprising a sequence set forth in SEQ ID NO: 30, a FR3 comprising a sequence set forth in SEQ ID NO: 82, and a FR4 comprising a sequence set forth in SEQ ID NO: 83;
(iv) a VL comprising a FR1 comprising a sequence set forth in SEQ ID NO: 84, a FR2 comprising a sequence set forth in SEQ ID NO: 85, a FR3 comprising a sequence set forth in SEQ ID NO: 86, and a FR4 comprising a sequence set forth in SEQ ID NO: 87; or
(v) a VH comprising a FR1 comprising a sequence set forth in SEQ ID NO: 80, a FR2 comprising a sequence set forth in SEQ ID NO: 30, a FR3 comprising a sequence set forth in SEQ ID NO: 82, and a FR4 comprising a sequence set forth in SEQ ID NO: 83 and a VL comprising a FR1 comprising a sequence set forth in SEQ ID NO: 84, a FR2 comprising a sequence set forth in SEQ ID NO: 85, a FR3 comprising a sequence set forth in SEQ ID NO: 86, and a FR4 comprising a sequence set forth in SEQ ID NO: 87.

As described herein, the antigen binding protein may be in the form of:
(i) a single chain Fv fragment (scFv);
(ii) a dimeric scFv (di-scFv);
(iii) one of (i) or (ii) linked to a constant region of an antibody, Fc or a heavy chain constant domain (CH) 2 and/or CH3, or
(iv) one of (i) or (ii) linked to a protein that binds to an immune effector cell.

Further, as described herein, the antigen binding protein may be in the form of:
(v) a diabody;
(vi) a triabody;
(vii) a tetrabody;
(viii) a Fab;
(ix) a F(ab′)2;
(x) a Fv;
(xi) one of (v) to (x) linked to a constant region of an antibody, Fc or a heavy chain constant domain (CH) 2 and/or CH3, or
(xii) one of (v) to (x) linked to a protein that binds to an immune effector cell.

The foregoing antigen binding proteins can also be referred to as antigen binding domains of antibodies.

Preferably, an antigen binding protein as described herein is an antibody or antigen binding fragment thereof. Typically, the antigen binding protein is an antibody, for example, a monoclonal antibody.

As used herein the antigen binding protein may be a variable domain.

The invention provides an antigen binding protein including, consisting essentially of or consisting of an amino acids sequence of (in order of N to C terminus or C to N terminus):
SEQ ID NO: 48 and 55;
SEQ ID NO: 49 and 56;
SEQ ID NO: 50 and 57;
SEQ ID NO: 51 and 58;
SEQ ID NO: 52 and 56;
SEQ ID NO: 53 and 55;
SEQ ID NO: 54 and 59;
SEQ ID NO: 88 and 89;
SEQ ID NO: 92 and 93;
SEQ ID NO: 96 and 98;
SEQ ID NO: 97 and 98.
SEQ ID NO: 88 and 98;
SEQ ID NO: 96 and 89; or
SEQ ID NO: 97 and 89.

In any aspect of the invention and in any antigen binding protein described herein, there further includes an Fc region that is engineered to have reduced capacity to induce antibody-dependent cell-mediated cytotoxicity (ADCC). Preferably, the reduced capacity to induce ADCC is conferred by mutation, deletion or modification of amino acids in the Fc region which interact with an Fc receptor. Preferably, the amino acids that are mutated, deleted or modified are at position 234, 235, and 331 as per SEQ ID NO:60 (where alanine is position 118) or at an equivalent position to 234, 235 and 331. Preferably, the amino acids are mutated to L234F, L235E and P331S. Typically, the Fc includes, consists essentially of or consists of an amino acid sequence shown in SEQ ID NO: 61.

As used herein, the complementarity determining region sequences (CDRs) of an antigen binding protein of the invention are defined according to the IMGT numbering system.

The invention provides an antigen binding protein as described herein wherein an amino acid sequence forming one or more of FR1, CDR1, FR2, CDR2, FR3, CDR3 and FR4 is a human sequence.

The invention provides an anti CCR6 receptor antigen binding protein, immunoglobulin variable domain, antibody, dab, scFv, Fab, Fab′, F(ab′)2, Fv fragment, diabody, triabody, linear antibody, single-chain antibody molecule, or multispecific antibody comprising an antigen binding protein having a sequence as described herein, or including a CDR and/or FR sequence as described herein.

The invention provides a diabody or triabody including an antigen binding protein having a sequence as described herein, or comprising a CDR and/or FR sequence as described herein.

The invention provides a fusion protein comprising an antigen binding protein, immunoglobulin variable domain, antibody, dab, scFv, Fab, Fab′, F(ab′)2, Fv fragment, diabody, triabody, linear antibody, single-chain antibody molecule, or multispecific antibody as described herein.

The invention provides a conjugate in the form of an antigen binding protein, immunoglobulin variable domain, antibody, dab, scFv, Fab, Fab′, F(ab′)2, Fv fragment, diabody, triabody, linear antibody, single-chain antibody molecule, or multispecific antibody or fusion protein as described herein conjugated to a label or a cytotoxic agent.

The invention provides an antibody for binding to an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein, or conjugate as described herein.

In aspects of the invention directed to multiple polypeptide chains that form an antigen binding protein, an expression construct comprises a nucleic acid encoding a polypeptide comprising, e.g., a VH operably linked to a promoter and a nucleic acid encoding a polypeptide comprising, e.g., a VL operably linked to a promoter.

In another example, the expression construct is a bicistronic expression construct, e.g., comprising the following operably linked components in 5′ to 3′ order:
(i) a promoter
(ii) a nucleic acid encoding a first polypeptide;
(iii) an internal ribosome entry site; and
(iv) a nucleic acid encoding a second polypeptide,
wherein the first polypeptide comprises a VH and the second polypeptide comprises a VL, or vice versa.

The present invention also contemplates separate expression constructs one of which encodes a first polypeptide comprising a VH and another of which encodes a second polypeptide comprising a VL. For example, the present invention also provides a composition comprising:
(i) a first expression construct comprising a nucleic acid encoding a polypeptide comprising a VH operably linked to a promoter; and
(ii) a second expression construct comprising a nucleic acid encoding a polypeptide comprising a VL operably linked to a promoter.

The invention provides a cell comprising a vector or nucleic acid described herein. Preferably, the cell is isolated, substantially purified or recombinant. In one example, the cell comprises the expression construct of the invention or:
(i) a first expression construct comprising a nucleic acid encoding a polypeptide comprising a VH operably linked to a promoter; and

(ii) a second expression construct comprising a nucleic acid encoding a polypeptide comprising a VL operably linked to a promoter,
wherein the first and second polypeptides associate to form an antigen binding protein of the present invention.

Examples of cells of the present invention include bacterial cells, yeast cells, insect cells or mammalian cells.

The invention provides a nucleic acid encoding an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein or conjugate as described herein. Preferably, the nucleic acid has a nucleotide sequence that encodes any one or more of the amino acid sequences corresponding to SEQ ID NO: 3 to 62 and 80 to 98. Preferably, the nucleic acid has a nucleotide sequence corresponding to any one or more of SEQ ID NO: 63 to 79, 99 or 100.

The invention provides a vector comprising a nucleic acid described herein. Preferably, the nucleic acid has a nucleotide sequence that encodes any one or more of the amino acid sequences corresponding to SEQ ID NO: 3 to 62 and 80 to 98. Preferably, the nucleic acid has a nucleotide sequence corresponding to any one or more of SEQ ID NOs: 63 to 79, or 99 or 100.

The invention provides a cell comprising a vector or nucleic acid described herein.

In another embodiment there is provided an animal or tissue derived therefrom comprising a cell described herein.

The invention provides a pharmaceutical composition comprising an antigen binding protein, or including a CDR and/or FR sequence as described herein, or an immunoglobulin variable domain, antibody, dab, scFv, Fab, Fab′, F(ab′)2, Fv fragment, diabody, triabody, linear antibody, single-chain antibody molecule, or multispecific antibody, fusion protein, or conjugate as described herein and a pharmaceutically acceptable carrier, diluent or excipient.

The invention provides a diagnostic composition comprising an antigen binding protein, or including a CDR and/or FR sequence as described herein, or antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein or conjugate as described herein, a diluent and optionally a label.

The invention provides a kit or article of manufacture comprising an antigen binding protein, or including a CDR and/or FR sequence as described herein or an immunoglobulin variable domain, antibody, dab, scFv, Fab, Fab′, F(ab′)2, Fv fragment, diabody, triabody, linear antibody, single-chain antibody molecule, or multispecific antibody, fusion protein or conjugate as described herein.

The invention provides use of a sequence according to one or more of CDR1, CDR2, FR1, FR2, FR3 and FR4 as described herein to produce an antigen binding protein for binding to a CCR6 receptor.

The invention provides use of an antigen binding protein or a CDR and/or FR sequence as described herein to produce an anti CCR6 receptor antigen binding protein having increased affinity for CCR6.

The invention provides a library of nucleic acid molecules produced from the mutation of an antigen binding protein or a CDR and/or FR sequence as described herein, wherein at least one nucleic acid molecule in said library encodes an antigen binding protein for binding to an a CCR6.

The invention provides a method for producing an antigen binding protein for binding to a CCR6 receptor as described herein comprising expressing a nucleic acid as described herein in a cell or animal as described herein.

The invention provides a method for the prevention or treatment a condition or disease associated with expression of CCR6 in an individual comprising the step of providing an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein, conjugate or pharmaceutical composition as described herein to an individual requiring treatment for said condition or disease. The disease or condition associated with expression of CCR6 may be an autoimmune or inflammatory condition, such as psoriasis, infection, fibrosis or cancer, especially an epithelial cancer as described herein, or pulmonary disorders such as Chronic obstructive pulmonary disease (COPD), asthma, and Respiratory syncytial virus (RSV).

The invention provides for a method for delaying onset or reducing severity of a condition or disease associated with expression of CCR6 in an individual comprising the step of providing an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein, conjugate or pharmaceutical composition as described herein to an individual requiring treatment for cancer or said condition or disease. Preferably, the disease is multiple sclerosis or psoriasis.

The invention provides for a method of preventing psoriasis or arthritis in an individual comprising the step of providing an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein, conjugate or pharmaceutical composition as described herein to an individual at risk of developing psoriasis or arthritis. Preferably, the psoriasis is plaque type psoriasis.

In any aspect of the present invention, the antigen binding protein comprises an Fc region that is engineered to have enhanced capacity to induce antibody-dependent cell-mediated cytotoxicity (ADCC). Preferably, the enhanced capacity to induce ADCC is conferred by mutation, deletion or modification of amino acids in the Fc region which interact with an Fc receptor. Preferably, the amino acids that are mutated, deleted or modified are at position 239, 330, and/or 332 as per SEQ ID NO: 60 (where alanine is position 118) or at an equivalent position to 239, 330 and/or 332. Preferably, the amino acids are mutated to S239D, A330L and I332E. Typically, the Fc comprises, consists essentially of or consists of an amino acid sequence shown in SEQ ID NO: 62.

In any aspect of the present invention, the antigen binding protein comprises an Fc region that is not engineered to have a reduced capacity to induce antibody-dependent cell mediated cytotoxicity (ADCC). Preferably, there the amino acids at position 234, 235, and/or 331 as per SEQ ID NO: 60 (where alanine is position 118) or at an equivalent position to 234, 235 and/or 331 are not F, E and/or S respectively. In other words, the amino acid at position 234 is not F, at position 235 is not E and/or at 331 is not S.

The invention provides for a method of preventing or treating psoriasis in an individual comprising the step of providing an antigen binding protein that inhibits the activity of CCR6 to an individual at risk of developing psoriasis or requiring treatment for psoriasis, wherein the antigen binding protein includes an Fc region that is engineered to have reduced capacity to induce antibody-dependent cell-mediated cytotoxicity (ADCC). Preferably, the reduced capacity to induce ADCC is conferred by mutation, deletion or modification of amino acids in the Fc region which interact with an Fc receptor. Preferably, the amino acids that are mutated, deleted or modified are at position 234, 235, and 331 as per SEQ ID NO:60 (where alanine is position 118) or at an equivalent position to 234, 235 and 331. Preferably, the amino acids are mutated to L234F, L235E and P331S. Typically, the Fc includes, consists essentially of or consists of an amino acid sequence shown in SEQ ID NO: 61. Preferably, the psoriasis is plaque type psoriasis.

The invention provides for a method of treating psoriasis in an individual comprising the step of providing an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein, conjugate or pharmaceutical composition as described herein to an individual requiring treatment for psoriasis. Preferably, the psoriasis is plaque type psoriasis.

The invention provides for a method of reducing the progression of psoriasis or arthritis in an individual comprising the step of providing an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein, conjugate or pharmaceutical composition as described herein to an individual requiring a reduction in the progression psoriasis. Preferably, the psoriasis is plaque type psoriasis.

The invention provides for a method of stabilizing or reversing the clinical symptoms of a condition or disease associated with expression of CCR6 in an individual comprising the step of providing an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein, conjugate or pharmaceutical composition as described herein to an individual requiring treatment for said condition or disease. Preferably, the disease is multiple sclerosis or psoriasis.

The invention provides for a method of treating a subject identified as having a symptom of a condition or disease associated with expression of CCR6 comprising administering an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein, conjugate or pharmaceutical composition as described herein, thereby treating the subject.

The invention provides use of an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein, conjugate or pharmaceutical composition as described herein in the manufacture of a medicament for the treatment of cancer or a condition or disease associated with expression of CCR6.

The invention provides a method for the diagnosis of a disease or condition associated with expression of CCR6, comprising the step of contacting tissues or cells for which the presence or absence of cancer is to be determined with a reagent in the form of an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein, conjugate or diagnostic composition as described herein and detecting for the binding of the reagent with the tissues or cells. The method may be operated in vivo or in vitro. The method may further include a step of treating a subject identified as having the disease or condition. Examples of diseases or conditions associated with expression of CCR6 are described herein.

In any aspect of the invention, the disease or condition associated with expression of CCR6 is arthritis. Arthritis may be any type, for example any type described herein including osteoarthritis, rheumatoid arthritis and psoriatic arthritis.

The invention provides antigen binding proteins according to the invention bind to CCR6 on live cells with affinities in the range of 0.1 to 5 nM.

An antigen binding protein, a protein or antibody as described herein may comprise a human constant region, e.g., an IgG constant region, such as an IgG1, IgG2, IgG3 or IgG4 constant region or mixtures thereof. In the case of an antibody or protein comprising a VH and a VL, the VH can be linked to a heavy chain constant region and the VL can be linked to a light chain constant region.

The functional characteristics of an antigen binding protein of the invention will be taken to apply mutatis mutandis to an antibody of the invention.

An antigen binding protein as described herein may be purified, substantially purified, isolated and/or recombinant.

An antigen binding protein of the invention may be part of a supernatant taken from media in which a hybridoma expressing an antigen binding protein of the invention has been grown.

The invention provides a single domain antibody comprising an antigen binding protein for binding to a CCR6.

As used herein, except where the context requires otherwise, the term “comprise” and variations of the term, such as “comprising”, “comprises” and “comprised”, are not intended to exclude further additives, components, integers or steps. The terms “comprising’ and “including” are used interchangeably.

Further aspects of the present invention and further embodiments of the aspects described in the preceding paragraphs will become apparent from the following description, given by way of example and with reference to the accompanying drawings.

### BRIEF DESCRIPTION OF THE SEQUENCES

SEQ ID NO: 1—Amino acid sequence of human CCR6.

SEQ ID NO: 2—Amino acid sequence of 1 to 28 of SEQ ID NO: 1.

SEQ ID NO: 3 to 62 and 80 to 98—Amino acid sequences described in Tables 1 to 7, below corresponding to CDR, FR and antigen binding domain sequences of the invention.

SEQ ID NOs: 63 to 79, 99 and 100—Nucleotide sequences of various VH, VL and Fc regions, as shown in Table 8.

### BRIEF DESCRIPTION OF THE DRAWINGS

FIGS. 1 and 2: Promising anti-CCR6 mAbs were initially ranked using competitive ligand binding and chemotaxis assays as described herein.

FIGS. 3 and 4: hCXCR1, hCXCR2, hCXCR3 transfectants were not stained by anti-CCR6 hybridoma culture supernatants.

FIG. 5: Anti-CCR6 mAbs grown in low IgG serum and partially purified were compared for ability to prevent ¹²⁵I-MIP3α binding to L1.2/hCCR6 transfectants. 53103 is R&D's anti-hCCR6 mAb.

FIG. 6: Anti-CCR6 mAbs grown in low IgG serum and partially purified were compared for ability to prevent MIP3α-induced migration of L1.2/hCCR6 transfectants 53103 is R&D's anti-hCCR6 mAb.

FIG. 7: Purified anti-CCR6 mAbs inhibited MIP-3α-induced migration of L1.2/hCCR6 transfectants.

FIG. 8: Epitope mapping of anti-hCCR6 mAb by ELISA as described herein.

FIG. 9: Staining human PBMC with AB6 and AB7 anti-CCR6 clones. Fresh human PBMC were gated on the lymphocytes population and stained with anti-CD3-FITC antibody and anti-CCR6-APC antibody (clone AB6 (a) and clone AB7 (b)).

FIG. 10: Humanization of anti-CCR6 (AB6) mAb by CDR grafting.

FIG. 11: Characterization of mouse AB6 mAb (mAB6) and humanized AB6 mAb (hAB6) by flow cytometry. mAB6 and hAB6 mAbs recognizes L1.2 cells transfected with hCCR6 (green line) but not untransfected L1.2 cells (black line).

FIG. 12: ADCC assay with purified human NK cells: L1.2 hCCR6 vs Fc engineered hAB6. Labeled target cells (hCCR6 L1.2-PK26) were incubated with purified human NK cells (effector cells) and 1 ug/ml of isotype control, non-depleting humanized AB6 (3SFc hAB6) or humanized depleting antibody (3MFc hAB6). Cells death was monitored with TO-PRO-3 iodide.

FIGS. 13 and 14: Generation human CCR6 knock-in mice (hCCR6+/mCCR6−/−). Staining of hCCR6+/mCCR6−/− derived splenocytes. Splenocytes were stained with anti-B220 APC mAb and hAB6 FITC (green), anti-mCCR6 (R&D; blue line) or isotype control (black line).

FIG. 15: Anti-human CCR6 mAb treatment of EAE using hCCR6+ knock-in mice. Eight to 12-week-old female hCCR6 Tg mice (5 per group) were injected subcutaneously with 100 μg rMOG 1-117 in complete Freund adjuvant. After immunization and 48 hours later, mice received an intravenous injection of 200 ng pertussis toxin. Individual animals were observed daily and clinical scores were assessed as follows: 0=no clinical disease, 1=loss of tail tone only, 2=mild monoparesis or paraparesis, 3=severe paraparesis, 4=paraplegia and/or quadraparesis, and 5=moribund or death. At day 8 (black arrow) mice received an IV injection of humanized non-depleting anti-hCCR6 antibody (hAB6) (2 mg/kg) or Isotype control (humanized anti-hCXCR3) antibody. * P<0.005, **P<0.001—ANOVA test.

FIG. 16: CCR6 depletion in mCCR6+/+/hCCR6+ mice. 350000 events were recorded in the lymphocyte population (splenocytes). mCCR6+/+/hCCR6+ mice were injected with hAB6 3MFc (d and f); hAB6 3SFc (b and g) or isotype control (c and e). #a) is a staining with an FITC isotype control antibody.

FIG. 17: Representative stained histological sections of spinal cords from immunized animal treated with isotype or anti-ccr6 mAb (preventive study). Serial sections were stained with hematoxylin and eosin (H&E) to determine the degree of inflammatory cell infiltrates, luxol fast blue (LFB: arrow heads) to establish myelin integrity and Bielschowski silver stain to ascertain for axonal loss and damage (arrows).

FIG. 18: Eight to 12-week-old female hCCR6 Tg mice were injected subcutaneously with 100 μg rMOG 1-117 in complete Freund adjuvant. After immunization and 48 hours later, mice received an intravenous injection of 200 ng pertussis toxin. When an average clinical score of 2 was reached (Day 15), the animals were treated with 2 mg/kg of humanized anti-hCCR6 or humanized anti-hCXCR3 mab (isotype group). Animals received a second injection on day 19. * P<0.005, **P<0.001—ANOVA test.

FIG. 19: Preventative psoriasis study. IMQ-induced skin inflammation in mice phenotypically resembles psoriasis (IMIQUIMOD-induced psoriasis model (The Journal of Immunology 2009 vol. 182 no. 9 5836-5845)). In a preventative study, hCCR6 Tg mice were treated daily with IMQ cream or control cream (vaseline) on the shaved back skin. (A) Phenotypical presentation of mouse back skin after 7 days of treatment. Mice were treated daily with Isotype control antibody (5 mg/kg) or humanized anti-hCCR6 mab, hAB6, (3Mfc or 3SFc at 5 mg/kg) beginning on the same day as the first application of IMQ cream. (B) IMQ treatment alters keratinocyte proliferation and differentiation. Mice were treated for 7 days with IMQ or vaseline cream. H&E staining of back skin of mice (Vaseline control; IMQ+Isotype or IMQ+hAB6 3MFc). (C) IMQ induced back skin thickening. Anti-hCCR6 significantly reduced back skin thickening.

FIG. 20: Therapeutic psoriasis study. The same IMQ model used in the experiments for FIG. 19 was employed, however mice were treated daily with isotype control antibody (5 mg/kg) or humanized anti-hCCR6 mab, hAB6, (3Mfc or 3SFc at 5 mg/kg) beginning on day 6 after the first application of IMQ cream. This therapeutic study measured IMQ induced back skin thickening with both anti-hCCR6 antibodies significantly reduced back skin thickening compared to control.

FIG. 21: In vitro ADCC assay. The cytolytic capacity of hAB6 depleting antibodies (IgG1 and IgG1 Fc optimised), was compared to non-depleting hAB6 (Fc KO) and isotype control. hAb6 depleting antibodies were shown to have significantly increased cell killing capacity compared to non-depleting or control.

FIG. 22: Therapeutic arthritis study. Top panel: Human CCR6 Transgenic mice were injected (i.p.) with 200 μL of K/BXN serum on day 0 and 1. The development of arthritis was assessed by measuring the ankle thickness and clinical index score every day until the experimental endpoint. When mice exhibiting symptoms of arthritis and cumulative clinical score reached 4 (on day 4), mice split were into two groups: those injected with isotype control mAb antibody; those that were injected with anti-hCCR6-FcKO antibody (blue); and those injected with anti-hCCR6-depleting antibody (green) at 20 mg/kg of body weight, followed by 5 mg/kg every other day for 1 week. As a control, mice that don't express human CCR6 (WT) were injected intraperitoneally with 200 μL of K/BXN serum on day 0 and 1 and treated with anti-hCCR6-depleting antibody (red). Bottom panel: Representative images of mouse ankles at experimental endpoint.

FIG. 23: FACS binding analysis of hAB6 (black curve) and hAB6 mutants (WT/3-3, blue curve; 1-21/WT, green curve; 1-23/WT, red curve; 1-21/3-3, orange curve; 1-23/3-3, pink curve) to hCCR6 L1.2 cells. Background fluorescence value was subtracted for each data point and plotted. EC50 was calculated using GraphPad Prism software.

FIG. 24: (a) Human CCR6 transgenic mice at age 6 weeks were acclimatized for 7 days at the animal house. Bleomycin (BLM) (Sigma) was diluted to 200 μg/ml with PBS. Bleomycin or PBS (100 μl) were injected subcutaneously into a single location on the shaved back of mice once daily for 28 days. Mice were treated subsequently by i.p. injections of anti-human CCR6 mAb, at 5 mg/kg, 3 times a week from day 8 until day 27. Control mice were treated by IP injections of Isotype control or PBS. (b) Dermal thickness shows increase thickness following BLM treatment and reduction of thickness when mice were further treated with anti-human CCR6 antibody.

FIG. 25: Histological evaluations. Skin (a) and lung tissues (b) from PBS-, isotype antibody- and anti-human CCR6-treated mice were formalin-fixed and embedded in paraffin. Sections were then stained with H&E; Masson's trichrome and picrosirius red for microscopic evaluation.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Further aspects of the present invention and further embodiments of the aspects described in the preceding paragraphs will become apparent from the following description, given by way of example and with reference to the accompanying drawings.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

The present inventors have developed antigen binding proteins, for example antibodies, that bind to and inhibit or reduce the activity of CCR6. The antigen binding proteins as described herein have the capacity to inhibit or reduce one or more aspects of the inflammatory, tumour growth and metastatic activity mediated by CCR6.

General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

Those skilled in the art will appreciate that the present invention is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

One skilled in the art will recognize many methods and materials similar or equivalent to those described herein, which could be used in the practice of the present invention. The present invention is in no way limited to the methods and materials described.

All of the patents and publications referred to herein are incorporated by reference in their entirety.

The present invention is not to be limited in scope by the specific examples described herein, which are intended for the purpose of exemplification only. Functionally-equivalent products, compositions and methods are clearly within the scope of the present invention.

Any example or embodiment of the present invention herein shall be taken to apply mutatis mutandis to any other example or embodiment of the invention unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901-917, 1987, Chothia et al. Nature 342, 877-883, 1989 and/or or Al-Lazikani et al., J Mol Biol 273, 927-948, 1997.

The term “and/or”, e.g., “X and/or Y” shall be understood to mean either “X and Y” or “X or Y” and shall be taken to provide explicit support for both meanings or for either meaning.

As used herein the term “derived from” shall be taken to indicate that a specified integer may be obtained from a particular source albeit not necessarily directly from that source.

Reference herein to a range of, e.g., residues, will be understood to be inclusive. For example, reference to “a region comprising amino acids 56 to 65” will be understood in an inclusive manner, i.e., the region comprises a sequence of amino acids as numbered 56, 57, 58, 59, 60, 61, 62, 63, 64 and 65 in a specified sequence.

### Selected Definitions

CCR6 is also known as C—X—C motif chemokine receptor 6 (CD196, BN-1, C—C CKR-6, CC-CKR-6, CCR-6, CD196, CKR-L3, CKRL3, CMKBR6, DCR2, DRY6, GPR29, GPRCY4, STRL22, C—C motif chemokine receptor 6). CCR6 1 is a G protein-coupled receptor (GPCR) that is expressed on many different cells and tissues, including lymphatic and non-lymphatic tissue such as spleen, lymph nodes, pancreas, colon, appendix, small intestine. CCR6 is expressed on B-cells, immature dendritic cells (DC), T-cells (Th1, Th2, Th17, Treg), natural killer cells (NKT cells) and neutrophils.

CCR6 binds with high affinity to CCL20, also known as macrophage protein 3 alpha (MIP3 alpha). Unlike other chemokine receptors, CCR6 does not bind to other chemokine ligands with a high degree of specificity.

Interleukin 4 (IL-4) and interferon gamma (IFNγ) suppress expression of CCR6 in Langerhans cells development and interleukin 10 (IL-10) induces its expression. Proinflammatory Th17 cells express CCR6 and its ligand CCL20 (MIP-3) and CCR6 influences migration of proinflammatory cells to sites of inflammation. Some Th17 cells migrate via a chemokine gradient of CCL20 (MIP-3) to inflammatory sites. In some models, the lack of CCR6 leads to less severe autoimmune encephalomyelitis.

CCR6 is also thought to have a function in the development and metastatic spread of gastrointestinal malignancies. Expression of CCR6 was found to be up-regulated in colorectal cancer. CCR6 has also been associated with Crohn's disease.

The term “CCR6” as provided herein includes any of the C—X—C motif chemokine receptor 6 (CCR6) protein naturally occurring forms, homologs or variants that maintain the activity of CCR6 (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In embodiments, the CCR6 protein is the protein as identified by the UniProt sequence reference P15684, homolog or functional fragment thereof.

For the purposes of nomenclature only and not a limitation, an exemplary amino acid sequence of human CCR6 is SEQ ID NO: 1.

As used herein, reference to CCR6 is to a molecule that has at least one biochemical or biophysical activity of CCR6. CCR6 biochemical or biophysical activities include acute inflammatory response to antigenic stimulus cell surface receptor signalling pathway, cellular defence response, chemotaxis, dendritic cell chemotaxis, inflammatory response, metanephric tubule morphogenesis, midbrain development, negative regulation of neutrophil apoptotic process, neutrophil activation, neutrophil chemotaxis, phospholipase C-activating G-protein coupled receptor signalling pathway, positive regulation of angiogenesis, positive regulation of cardiac muscle cell apoptotic process, positive regulation of cell proliferation, positive regulation of cytosolic calcium ion concentration positive regulation of neutrophil chemotaxis, positive regulation of vascular permeability, receptor internalization, and signal transduction

The phrase “inhibits CCR6 activity” is understood to mean that the antigen binding protein of the present invention inhibits or reduces any one or more activities of CCR6, including but not limited to ligand binding to CCR6; ligand induced conformational change of CCR6; CCR6 activation; G protein activation; CCR6 mediated cell signalling; a CCR6 mediated cell migratory, inflammatory, tumour growth, angiogenic or metastatic response in vitro or in vivo; CCR6 mediated tumour cell growth; and/or CCR6 mediated leukocyte (e.g. neutrophil, eosinophil, mast cell or T cell) migration. “Inhibits MIP-3 mediated CCR6 activity” is understood to mean that the antigen binding protein of the present invention inhibits or reduces one or more activities described above that are mediated or induced by MIP-3. Further, the activity is measured using a suitable in vitro, cellular or in vivo assay and the activity is blocked or reduced by at least 1%, 5%, 10%, 25%, 50%, 60%, 70%, 80% or 90% or more, compared to CCR6 activity in the same assay under the same conditions but without the antigen binding protein. Preferably, the CCR6 activity is mediated or induced by MIP3.

SEQ ID NO: 1:
MSGESMNFSDVFDSSEDYFVSVNTSYYSVDSEMLLCSLQEVRQFSRLFVPI
AYSLICVFGLLGNILVVITFAFYKKARSMTDVYLLNMAIADILFVLTLPFW
AVSHATGAWVFSNATCKLLKGIYAINFNCGMLLLTCISMDRYIAIVQATKS
FRLRSRTLPRSKIICLWWWGLSVIISSSTFVFNQKYNTQGSDVCEPKYQTV
SEPIRWKLLMLGLELLFGFFIPLMFMIFCYTFIVKTLVQAQNSKRHKAIRV
IIAVVLVFLACQIPHNMVLLVTAANLGKMNRSCQSEKLIGYTKTVTEVLAF
LHCCLNPVLYAFIGQKFRNYFLKILKDLWCVRRKYKSSGFSCAGRYSENIS
RQTSETADNDNASSFTM

The term “isolated protein” or “isolated polypeptide” is a protein or polypeptide that by virtue of its origin or source of derivation is not associated with naturally-associated components that accompany it in its native state; is substantially free of other proteins from the same source. A protein may be rendered substantially free of naturally associated components or substantially purified by isolation, using protein purification techniques known in the art. By “substantially purified” is meant the protein is substantially free of contaminating agents, e.g., at least about 70% or 75% or 80% or 85% or 90% or 95% or 96% or 97% or 98% or 99% free of contaminating agents.

The term “recombinant” shall be understood to mean the product of artificial genetic recombination. Accordingly, in the context of a recombinant protein comprising an antibody antigen binding domain, this term does not encompass an antibody naturally-occurring within a subject's body that is the product of natural recombination that occurs during B cell maturation. However, if such an antibody is isolated, it is to be considered an isolated protein comprising an antibody antigen binding domain. Similarly, if nucleic acid encoding the protein is isolated and expressed using recombinant means, the resulting protein is a recombinant protein comprising an antibody antigen binding domain. A recombinant protein also encompasses a protein expressed by artificial recombinant means when it is within a cell, tissue or subject, e.g., in which it is expressed.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulphide bond. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

As used herein, the term “antigen binding protein” is used interchangeably with “antigen binding domain” and shall be taken to mean a region of an antibody that is capable of specifically binding to an antigen, i.e., a VH or a VL or an Fv comprising both a VH and a VL. The antigen binding domain need not be in the context of an entire antibody, e.g., it can be in isolation (e.g., a domain antibody) or in another form, e.g., as described herein, such as a scFv.

For the purposes for the present disclosure, the term “antibody” includes a protein capable of specifically binding to one or a few closely related antigens (e.g., CCR6) by virtue of an antigen binding domain contained within a Fv. This term includes four chain antibodies (e.g., two light chains and two heavy chains), recombinant or modified antibodies (e.g., chimeric antibodies, humanized antibodies, human antibodies, CDR-grafted antibodies, primatized antibodies, de-immunized antibodies, synhumanized antibodies, half-antibodies, bispecific antibodies). An antibody generally comprises constant domains, which can be arranged into a constant region or constant fragment or fragment crystallizable (Fc). Exemplary forms of antibodies comprise a four-chain structure as their basic unit. Full-length antibodies comprise two heavy chains (˜50 to 70 kD) covalently linked and two light chains (˜23 kDa each). A light chain generally comprises a variable region (if present) and a constant domain and in mammals is either a K light chain or a λ light chain. A heavy chain generally comprises a variable region and one or two constant domain(s) linked by a hinge region to additional constant domain(s). Heavy chains of mammals are of one of the following types α, δ, ε, γ, or μ. Each light chain is also covalently linked to one of the heavy chains. For example, the two heavy chains and the heavy and light chains are held together by inter-chain disulfide bonds and by non-covalent interactions. The number of inter-chain disulfide bonds can vary among different types of antibodies. Each chain has an N-terminal variable region (VH or VL wherein each are ˜110 amino acids in length) and one or more constant domains at the C-terminus. The constant domain of the light chain (CL which is ˜110 amino acids in length) is aligned with and disulfide bonded to the first constant domain of the heavy chain (CH1 which is 330 to 440 amino acids in length). The light chain variable region is aligned with the variable region of the heavy chain. The antibody heavy chain can comprise 2 or more additional CH domains (such as, CH2, CH3 and the like) and can comprise a hinge region between the CH1 and CH2 constant domains. Antibodies can be of any type (e.g., IgG, IgE, IgM, IgD, IgA, and IgY), class (e.g., IgG1, IgG2, IgG3, IgG4, IgA1 and IgA2) or subclass. In one example, the antibody is a murine (mouse or rat) antibody or a primate (such as, human) antibody. In one example the antibody heavy chain is missing a C-terminal lysine residue. In one example, the antibody is humanized, synhumanized, chimeric, CDR-grafted or deimmunized.

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that is capable of specifically binding to an antigen and, includes amino acid sequences of complementarity determining regions (CDRs); i.e., CDR1, CDR2, and CDR3, and framework regions (FRs). For example, the variable region comprises three or four FRs (e.g., FR1, FR2, FR3 and optionally FR4) together with three CDRs. VH refers to the variable region of the heavy chain. VL refers to the variable region of the light chain.

As used herein, the term “epitope” (syn. “antigenic determinant”) shall be understood to mean a region of CXCR6 to which an antigen binding protein comprising an antigen binding domain of an antibody binds. Unless otherwise defined, this term is not necessarily limited to the specific residues or structure to which the antigen binding protein makes contact. For example, this term includes the region spanning amino acids contacted by the antigen binding protein and 5-10 (or more) or 2-5 or 1-3 amino acids outside of this region. In some examples, the epitope comprises a series of discontinuous amino acids that are positioned close to one another when antigen binding protein is folded, i.e., a “conformational epitope”. The skilled artisan will also be aware that the term “epitope” is not limited to peptides or polypeptides. For example, the term “epitope” includes chemically active surface groupings of molecules such as sugar side chains, phosphoryl side chains, or sulfonyl side chains, and, in certain examples, may have specific three dimensional structural characteristics, and/or specific charge characteristics.

As used herein, the term “subject” shall be taken to mean any animal including humans, for example a mammal. Exemplary subjects include but are not limited to humans and non-human primates. For example, the subject is a human.

The present inventors have developed antibodies that bind to and inhibit the function of CCR6. The antibodies have high affinity for CCR6 and inhibit ligand (MIP-3α) mediated chemotaxis. These antibodies have been shown to bind CCR6 as it is naturally presented on the surface of immune cells. In view of the properties of the antibodies are described herein, including the Examples, these antibodies are useful for delaying onset or reducing severity of diseases associated with MIP-3α activity or CCR6 expression. Further, these antibodies have been shown to stabilize and reverse clinically observable symptoms of established disease associated with MIP-3α activity or CCR6 expression

“Antibodies” or “immunoglobulins” or “Igs” are gamma globulin proteins that are found in blood, or other bodily fluids of vertebrates that function in the immune system to bind antigen, hence identifying and neutralizing foreign objects.

Antibodies are generally a heterotetrameric glycoprotein composed of two identical light (L) chains and two identical heavy (H) chains. Each L chain is linked to a H chain by one covalent disulfide bond. The two H chains are linked to each other by one or more disulfide bonds depending on the H chain isotype. Each H and L chain also has regularly spaced intrachain disulfide bridges.

H and L chains define specific Ig domains. More particularly, each H chain has at the N-terminus, a variable domain (VH) followed by three constant domains (CH) for each of the α and γ chains and four CH domains for μ and ε isotypes. Each L chain has at the N-terminus, a variable domain (V L) followed by a constant domain (CL) at its other end. The VL is aligned with the VH and the CL is aligned with the first constant domain of the heavy chain (CH1).

Antibodies can be assigned to different classes or isotypes. There are five classes of immunoglobulins: IgA, IgD, IgE, IgG, and IgM, having heavy chains designated α, δ, ε, γ, and μ, respectively. The γ and α classes are further divided into subclasses on the basis of relatively minor differences in CH sequence and function, e.g., humans express the following subclasses: IgG1, IgG2, IgG3, IgG4, IgA1, and IgA2. The L chain from any vertebrate species can be assigned to one of two clearly distinct types, called kappa and lambda, based on the amino acid sequences of their constant domains.

The constant domain includes the Fc portion which comprises the carboxy-terminal portions of both H chains held together by disulfides. The effector functions of antibodies such as ADCC are determined by sequences in the Fc region, which region is also the part recognized by Fc receptors (FcR) found on certain types of cells.

The pairing of a VH and VL together forms a “variable region” or “variable domain” including the amino-terminal domains of the heavy or light chain of the antibody. The variable domain of the heavy chain may be referred to as “VH.” The variable domain of the light chain may be referred to as “VL.” The V domain contains an antigen binding protein which affects antigen binding and defines specificity of a particular antibody for its particular antigen. V R&D regions span about 110 amino acid residues and consist of relatively invariant stretches called framework regions (FRs) (generally about 4) of 15-30 amino acids separated by shorter regions of extreme variability called “hypervariable regions” (generally about 3) that are each 9-12 amino acids long. The FRs largely adopt a β-sheet configuration and the hypervariable regions form loops connecting, and in some cases forming part of, the β-sheet structure.

“Hypervariable region”, “HVR”, or “HV” refers to the regions of an antibody variable domain which are hypervariable in sequence and/or form structurally defined loops. Generally, antibodies comprise six hypervariable regions; three in the VH (H1, H2, H3), and three in the VL (L1, L2, L3). A number of hypervariable region delineations are in use and are encompassed herein.

As used herein, the term “complementarity determining regions” (syn. CDRs; i.e., CDR1, CDR2, and CDR3) refers to the amino acid residues of an antibody variable region the presence of which are major contributors to specific antigen binding. Each variable region domain (VH or VL) typically has three CDRs identified as CDR1, CDR2 and CDR3. The CDRs of VH are also referred to herein as CDR H1, CDR H2 and CDR H3, respectively, wherein CDR H1 corresponds to CDR 1 of VH, CDR H2 corresponds to CDR 2 of VH and CDR H3 corresponds to CDR 3 of VH. Likewise, the CDRs of VL are referred to herein as CDR L1, CDR L2 and CDR L3, respectively, wherein CDR L1 corresponds to CDR 1 of VL, CDR L2 corresponds to CDR 2 of VL and CDR L3 corresponds to CDR 3 of VL. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system”). In another example, the amino acid positions assigned to CDRs and FRs are defined according to the Enhanced Chothia Numbering Scheme (http://www.bioinfo.org.uk/mdex.html). The present invention is not limited to FRs and CDRs as defined by the Kabat numbering system, but includes all numbering systems, including the canonical numbering system or of Chothia and Lesk J. Mol. Biol. 196: 901-917, 1987; Chothia et al., Nature 342: 877-883, 1989; and/or Al-Lazikani et al., J. Mol. Biol. 273: 927-948, 1997; the numbering system of Honnegher and Plükthun J. Mol. Biol. 309: 657-670, 2001; or the IMGT system discussed in Giudicelli et al., Nucleic Acids Res. 25: 206-211 1997. In one example, the CDRs are defined according to the Kabat numbering system. Optionally, heavy chain CDR2 according to the Kabat numbering system does not comprise the five C-terminal amino acids listed herein or any one or more of those amino acids are substituted with another naturally-occurring amino acid. In this regard, Padlan et al., FASEB J., 9: 133-139, 1995 established that the five C-terminal amino acids of heavy chain CDR2 are not generally involved in antigen binding.

“Framework” or “FR” residues are those variable domain residues other than the hypervariable region or CDR residues herein defined. The FRs of VH are also referred to herein as FR H1, FR H2, FR H3 and FR H4, respectively, wherein FR H1 corresponds to FR 1 of VH, FR H2 corresponds to FR 2 of VH, FR H3 corresponds to FR 3 of VH and FR H4 corresponds to FR 4 of VH. Likewise, the FRs of VL are referred to herein as FR L1, FR L2, FR L3 and FR L4, respectively, wherein FR L1 corresponds to FR 1 of VL, FR L2 corresponds to FR 2 of VL, FR L3 corresponds to FR 3 of VL and FR L4 corresponds to FR 4 of VL.

“A peptide for forming an antigen binding protein” generally refers to a peptide that may form a conformation that confers the specificity of an antibody for antigen. Examples include whole antibody or whole antibody related structures, whole antibody fragments including a variable domain, variable domains and fragments thereof, including light and heavy chains, or fragments of light and heavy chains that include some but not all of hypervariable regions or constant regions.

An “intact” or “whole” antibody is one which comprises an antigen-binding protein as well as a CL and at least heavy chain constant domains, CHI, CH2 and CH3. The constant domains may be native sequence constant domains (e.g. human native sequence constant domains) or amino acid sequence variant thereof.

“Whole antibody related structures” include multimerized forms of whole antibody.

“Whole antibody fragments including a variable domain” include Fab, Fab′, F(ab′)2, and Fv fragments; diabodies; linear antibodies, single-chain antibody molecules; and multispecific antibodies formed from antibody fragments.

The Fab fragment consists of an entire L chain along with the variable region domain of the H chain (VH), and the first constant domain of one heavy chain (CHI). Each Fab fragment is monovalent with respect to antigen binding, i.e., it has a single antigen-binding protein.

A Fab′ fragment differs from Fab fragments by having additional few residues at the carboxy terminus of the CHI domain including one or more cysteines from the antibody hinge region. Fab′-SH is the designation herein for Fab′ in which the cysteine residue(s) of the constant domains bear a free thiol group.

A F(ab′)2 fragment roughly corresponds to two disulfide linked Fab fragments having divalent antigen-binding activity and is still capable of cross-linking antigen.

An “Fv” is an antibody fragment which contains a complete antigen-recognition and -binding site. This fragment consists of a dimer of one heavy- and one light-chain variable region domain in tight, non-covalent association.

In a single-chain Fv (scFv) species, one heavy- and one light-chain variable domain can be covalently linked by a flexible peptide linker such that the light and heavy chains can associate in a “dimeric” structure analogous to that in a two-chain Fv species. From the folding of these two domains emanate six hypervariable loops (3 loops each from the H and L chain) that contribute the amino acid residues for antigen binding and confer antigen binding specificity to the antibody.

“Single-chain Fv” also abbreviated as “sFv” or “scFv” are antibody fragments that comprise the VH and VL antibody domains connected to form a single polypeptide chain. Preferably, the scFv polypeptide further comprises a polypeptide linker between the VH and VL domains which enables the scFv to form the desired structure for antigen binding.

A “single variable domain” is half of an Fv (comprising only three CDRs specific for an antigen) that has the ability to recognize and bind antigen, although at a lower affinity than the entire binding site.

“Diabodies” refers to antibody fragments with two antigen-binding sites, which fragments comprise a heavy-chain variable domain (VH) connected to a light-chain variable domain (VL) in the same polypeptide chain (VH-VL). The small antibody fragments are prepared by constructing sFv fragments (see preceding paragraph) with short linkers (about 5-10 residues) between the VH and VL domains such that interchain but not intra-chain pairing of the V domains is achieved, resulting in a bivalent fragment, i.e., fragment having two antigen-binding sites.

Diabodies may be bivalent or bispecific. Bispecific diabodies are heterodimers of two “crossover” sFv fragments in which the VH and VL domains of the two antibodies are present on different polypeptide chains. Triabodies and tetrabodies are also generally known in the art.

An “isolated antibody” is one which has been identified and separated and/or recovered from a component of its pre-existing environment. Contaminant components are materials that would interfere with therapeutic uses for the antibody, and may include enzymes, hormones, and other proteinaceous or nonproteinaceous solutes.

A “human antibody” refers to an antibody which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Human antibodies can be prepared by administering the antigen to a transgenic animal that has been modified to produce such antibodies in response to antigenic challenge, but whose endogenous loci have been disabled.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin.

“Monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site or determinant on the antigen. In addition to their specificity, the monoclonal antibodies are advantageous in that they may be synthesized uncontaminated by other antibodies. Monoclonal antibodies may be prepared by the hybridoma methodology, or may be made using recombinant DNA methods in bacterial, eukaryotic animal or plant cells. The “monoclonal antibodies” may also be isolated from phage antibody libraries.

The monoclonal antibodies herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. Chimeric antibodies of interest herein include “primatized” antibodies comprising variable domain antigen-binding sequences derived from a non-human primate (e.g. Old World Monkey, Ape etc), and human constant region sequences.

The term “anti-CCR6 antibody” or “an antibody that binds to CCR6” refers to an antibody that is capable of binding CCR6 with sufficient affinity such that the antibody is useful as a diagnostic and/or therapeutic agent in targeting CCR6. Preferably, the extent of binding of a CCR6 antibody to an unrelated receptor protein is less than about 10% of the binding of the antibody to CCR6 as measured, e.g., by a radioimmunoassay (RIA). In certain embodiments, an antibody that binds to CCR6 has a dissociation constant (Kd) of <1 μM, <100 nM, <10 nM, <1 nM, or <0.1 nM.

“Binding affinity” generally refers to the strength of the sum total of noncovalent interactions between a single binding site of a molecule (e.g., an antibody) and its binding partner (e.g., an antigen). Generally, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention.

As used herein, the term “binds” in reference to the interaction of an antigen binding protein or an antigen binding domain thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabelled “A”), in a reaction containing labeled “A” and the protein, will reduce the amount of labelled “A” bound to the antibody.

As used herein, the term “specifically binds” or “binds specifically” shall be taken to mean that an antigen binding protein of the invention reacts or associates more frequently, more rapidly, with greater duration and/or with greater affinity with a particular antigen or cell expressing same than it does with alternative antigens or cells. For example, an antigen binding protein binds to CCR6 (e.g., hCCR6) with materially greater affinity (e.g., 1.5 fold or 2 fold or 5 fold or 10 fold or 20 fold or 40 fold or 60 fold or 80 fold to 100 fold or 150 fold or 200 fold) than it does to other CCRs. In an example of the present invention, an antigen binding protein that “specifically binds” to CCR6 (preferably human) with an affinity at least 1.5 fold or 2 fold or greater (e.g., 5 fold or 10 fold or 20 fold or 50 fold or 100 fold or 200 fold) than it does to another chemokine receptor, such as CXCR1, CXCR2, CXCR3, or CXCR7. Generally, but not necessarily, reference to binding means specific binding, and each term shall be understood to provide explicit support for the other term.

As used herein, the term “does not detectably bind” shall be understood to mean that an antigen binding protein, e.g., an antibody, binds to a candidate antigen at a level less than 10%, or 8% or 6% or 5% above background. The background can be the level of binding signal detected in the absence of the protein and/or in the presence of a negative control protein (e.g., an isotype control antibody) and/or the level of binding detected in the presence of a negative control antigen. The level of binding is detected using biosensor analysis (e.g. Biacore) in which the antigen binding protein is immobilized and contacted with an antigen.

As used herein, the term “does not significantly bind” shall be understood to mean that the level of binding of an antigen binding protein of the invention to a polypeptide is not statistically significantly higher than background, e.g., the level of binding signal detected in the absence of the antigen binding protein and/or in the presence of a negative control protein (e.g., an isotype control antibody) and/or the level of binding detected in the presence of a negative control polypeptide. The level of binding is detected using biosensor analysis (e.g. Biacore) in which the antigen binding protein is immobilized and contacted with an antigen.

An “affinity matured” antibody is one with one or more alterations in one or more HVRs thereof which result in an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art.

“ADCC” refers to a process called antibody-dependent cellular cytotoxicity, which is an immune response mediated primarily by natural killer (NK) cells in humans. In ADCC, FcγRIII on the surface of an NK cell recognizes the Fe region of antibody that is bound to antigen displayed on the surface of a target cell. This activates the NK cell, which releases perforins and granzymes, leading to lysis and apoptosis of the target cells.

“CDC” refers to a complex process called complement-dependent cytotoxicity that can lead to cell killing through the action of a cascade of proteins that can act through either of two major pathways.

“ADCP” refers to a process called antibody dependent cell-mediated phagocytosis. In this Fe receptor-mediated process, target cells to which antibodies are bound are engulfed by phagocytic cells, such as macrophage, monocytes, neutrophils, and dendritic cells. Multiple Fc receptors are involved in this process.

A “blocking” antibody or an “antagonist” antibody is one which inhibits or reduces biological activity of the antigen it binds. Preferred blocking antibodies or antagonist antibodies substantially or completely inhibit the biological activity of the antigen.

An “agonist antibody”, as used herein, is an antibody which mimics at least one of the functional activities of a polypeptide of interest.

As meant herein, an “Fc region” is a dimer consisting of two polypeptide chains joined by one or more disulfide bonds, each chain comprising part or all of a hinge domain plus a CH2 and a CH3 domain. Each of the polypeptide chains is referred to as an “Fc polypeptide chain.” To distinguish the two Fe polypeptide chains, one is referred to herein as an “A chain” and the other is referred to as a “B chain.” More specifically, the Fc regions contemplated for use with the present invention are IgG Fc regions, which can be mammalian or human IgG1, IgG2, IgG3, or IgG4 Fc regions. Among human IgG1 Fc regions, at least two allelic types are known.

An “Fc-containing protein,” as meant herein, is a protein comprising an Fc region as described herein and a binding region that binds to a target molecule. The term “Fc containing protein” encompasses an antibody or an Fc fusion protein that contains an Fc region.

A “disease or condition associated with CCR6 expression” include, but are not limited to, an inflammatory condition as described herein, an autoimmune disease as described herein, an infection, fibrosis or a cancer, especially an epithelial cancer as described herein, or pulmonary disorders such as Chronic obstructive pulmonary disease (COPD), asthma, and Respiratory syncytial virus (RSV). Other diseases or conditions are described further herein.

The phrase “therapeutically effective amount” generally refers to an amount of a antigen binding protein of the present invention that (i) treats the particular disease, condition, or disorder, (ii) attenuates, ameliorates, or eliminates one or more symptoms of the particular disease, condition, or disorder, or (iii) delays the onset of one or more symptoms of the particular disease, condition, or disorder described herein.

The words “treat” or “treatment” refer to therapeutic treatment wherein the object is to slow down (lessen) an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. ‘Treatment can also mean prolonging survival as compared to expected survival if not receiving treatment. Treatment may not necessarily result in the complete clearance of a disease or disorder but may reduce or minimise complications and side effects of infection and the progression of a disease or disorder. The success or otherwise of treatment may be monitored by, amongst other things, physical examination of the individual, cytopathological, serological DNA, or mRNA detection techniques.

Treatment of psoriasis may be observed or measured by a reduction in the severity of, or reversal of, any one or more of the clinically or biochemically observable or measurable characteristics of psoriasis including plaques, hyperproliferative keratinocytes, a disturbed epidermal differentiation (parakeratosis) which is commonly displayed by the retention of nuclei in the stratum corneum, the absence of a granular layer, an altered involucrin expression pattern, epidermal thickening, erythema, scaling, or any other characteristic described herein.

The words “prevent” and “prevention” generally refer to prophylactic or preventative measures for protecting or precluding an individual not having a given disease or disorder from progressing to that disease or disorder.

An individual at risk of developing psoriasis may be identified by a medical practitioner based on known biochemical and clinical susceptibility indicators.

The phrase “pharmaceutically acceptable” indicates that the substance or composition must be compatible chemically and/or toxicologically, with the other ingredients comprising a formulation, and/or the mammal being treated therewith.

The inventors have determined the CDR sequences of a number of variable domain clones that they have found to bind to CCR6. These CDR sequences are shown in Table 1 below.

In one embodiment there is provided a peptide having a sequence as shown in Tables 1 and 2. These peptides are particularly useful for constructing antigen binding proteins, variable domains, antibodies and related fragments.

TABLE 1
VH CDR sequences

| Clone | CDR1 | CDR2 | CDR3 |
|---|---|---|---|
| AB1 | SEQ ID NO: 3 | SEQ ID NO: 4 | SEQ ID NO: 5 |
| | GFTFSDYY | ITNGDGRT | TSPPLGGAWFGY |
| AB2 | SEQ ID NO: 6 | SEQ ID NO: 7 | SEQ ID NO: 5 |
| | GFSFSDYY | ITNGGGRT | TSPPLGGAWFGY |
| AB3 | SEQ ID NO: 3 | SEQ ID NO: 7 | SEQ ID NO: 8 |
| | GFTFSDYY | ITNGGGRT | ASPPLGGAWFGY |
| AB4 | SEQ ID NO: 3 | SEQ ID NO: 7 | SEQ ID NO: 8 |
| | GFTFSDYY | ITNGGGRT | ASPPLGGAWFGY |
| AB5 | SEQ ID NO: 3 | SEQ ID NO: 9 | SEQ ID NO: 10 |
| | GFTFSDYY | ITNGAGRT | ASPPLGGAWFAY |
| AB6 | SEQ ID NO: 11 | SEQ ID NO: 12 | SEQ ID NO: 13 |
| | GFPFSDYY | ITNGVGRT | TSPPLGGAWFAY |
| AB7 | SEQ ID NO: 14 | SEQ ID NO: 15 | SEQ ID NO: 16 |
| | EYTFKSFG | IYPRSGNT | ARSPYDGYFDY |
| AB11 | SEQ NO: 6 | SEQ ID NO: 7 | SEQ ID NO: 5 |
| | GFSFSDYY | ITNGGGRT | TSPPLGGAWFGY |
| 1-21 | SEQ ID NO: 11 | SEQ ID NO: 12 | SEQ ID NO: 5 |
| & 1-23 | GFPFSDYY | ITNGVGRT | TSPPLGGAWFGY |

TABLE 2
VL CDR sequences

| Clone | CDR1 | CDR2 | CDR3 |
|---|---|---|---|
| AB1 | SEQ ID NO: 17 | SEQ ID NO: 18 | SEQ ID NO: 19 |
| | QSIVHSNGNTY | KVS | FQGSHVPLT |
| AB2 | SEQ ID NO: 20 | SEQ ID NO: 18 | SEQ ID NO: 19 |
| | QSLVHSNGNTY | KVS | FQGSHVPLT |
| AB3 | SEQ ID NO: 17 | SEQ ID NO: 18 | SEQ ID NO: 19 |
| | QSIVHSNGNTY | KVS | FQGSHVPLT |
| AB4 | SEQ ID NO: 20 | SEQ ID NO: 18 | SEQ ID NO: 19 |
| | QSLVHSNGNTY | KVS | FQGSHVPLT |
| AB5 | SEQ ID NO: 20 | SEQ ID NO: 18 | SEQ ID NO: 19 |
| | QSLVHSNGNTY | KVS | FQGSHVPLT |
| AB6 | SEQ ID NO: 17 | SEQ ID NO: 18 | SEQ ID NO: 19 |
| | QSIVHSNGNTY | KVS | FQGSHVPLT |
| AB7 | SEQ ID NO: 21 | SEQ ID NO: 22 | SEQ ID NO: 23 |
| | QSLLHINGNTY | RVS | SQSTHVPRT |
| AB11 | SEQ ID NO: 20 | SEQ ID NO: 18 | SEQ ID NO: 19 |
| | QSLVHSNGNTY | KVS | FQGSHVPLT |
| 3-3 | SEQ ID NO: 94 | SEQ ID NO: 18 | SEQ ID NO: 19 |
| | RSIVHSNGNTY | KVS | FQGSHVPLT |

The inventors have determined the FR sequences of a number of variable domain clones that they have found to bind to CCR6. These FR sequences are shown in Table 3 and 4 below. Other known FR sequences could be used with the above described CDRs to form an antigen binding protein for binding to a CCR6.

TABLE 3
VH Framework regions

| Clone | FR1 |
|---|---|
| AB1, AB3, AB4 | SEQ ID NO: 24 |
| | EVNLVESGGGLVQPGGSLKLSCAAS |
| AB2, AB5 | SEQ ID NO: 25 |
| | EVNLVESGGGLVQPGGSLKLSCEAS |
| AB6 | SEQ ID NO: 26 |
| | EVKLVESGGGLVQPGGSLKLSCAAS |
| AB7 | SEQ ID NO: 27 |
| | QDQLQQSGVALARPGASVKLSCKAS |
| hAB6, 1-21 | SEQ ID NO: 80 |
| | EVQLVESGGGLVQPGGSLRLSCAAS |
| AB11 | SEQ ID NO: 90 |
| | EVNLVESGGGLVQPGGSLILSCEAS |
| 1-23 | SEQ ID NO: 95 |
| | EVQLVESGGGLVQPGGSLRLSCVAS |
| Clone | FR2 |
| AB1, AB2, AB4, | SEQ ID NO: 28 |
| AB5, AB11 | MYWVRQTPEKRLEWVTY |
| AB3 | SEQ ID NO: 29 |
| | LYWVRQTPEKRLEWVTY |
| AB6 | SEQ ID NO: 30 |
| | LYWVRQTPEKRLEWVAY |
| AB7 | SEQ ID NO: 31 |
| | LGWVKQRPGQGLEWIGE |
| hAB6, 1-21, | SEQ ID NO: 81 |
| 1-23 | LYWVRQAPGKGLEWVAY |
| Clone | FR3 |
| AB1, AB3, | SEQ ID NO: 32 |
| AB4, AB5 | YYSDTVRGRFTISRDNAKNTLYLQMSRLKSED |
| | TAMYYC |
| AB2 | SEQ ID NO: 33 |
| | YYSDTIRGRFTISRDNARNTLYLQMSRLKSED |
| | TAMYYC |
| AB6, 1-21, | SEQ ID NO: 34 |
| 1-23 | YYSDSVKGRFTISRDNAKNTLYLQMSRLKSED |
| | TSMYYC |
| AB7 | SEQ ID NO: 35 |
| | YYNEKVKGKVRLTADKSSNSVYMEFRSLTSED |
| | SAVYFC |
| hAB6 | SEQ ID NO: 82 |
| | YYSDSVKGRFTISRDNAKNTLYLQMNSLRDED |
| | TAVYYC |
| AB11 | SEQ ID NO: 91 |
| | YYSDAIRGRFTISRDNARNTLYLQMSRLKSE |
| | TAMYYC |
| Clone | FR4 |
| AB1, AB2, AB3, | SEQ ID NO: 36 |
| AB4, AB5, AB6, | WGQGTLVTVS |
| AB11 | |
| AB7 | SEQ ID NO: 37 |
| | WGQGTTLTVS |
| hAB6, 1-21, | SEQ ID NO: 83 |
| 1-23 | WGQGTLVTVS |

TABLE 4
V_{L} Framework regions

| Clone | FR1 |
|---|---|
| AB1, AB6, AB3, AB4 | SEQ ID NO: 38 |
| | DVLMTQTPLSLPVSLGDQASISCRSS |
| AB2, AB5, AB11 | SEQ ID NO: 39 |
| | DVSMTQTPLSLPVSLGDQASISCRSS |
| AB7 | SEQ ID NO: 40 |
| | DVVMTHSPLSLPVSLGDQASISCRSS |
| hAB6, 3-3 | SEQ ID NO: 84 |
| | DIVMTQSPLSLPVTPGEPASISCRSS |
| Clone | FR2 |
| AB1, AB3, AB4, AB5, | SEQ ID NO: 41 |
| AB2, AB6, AB11 | LEWYLQKPGQSPKLLIY |
| AB7 | SEQ ID NO: 42 |
| | LHWYLQKPGQSPKLLIY |
| hAB6, 3-3 | SEQ ID NO: 85 |
| | LEWYLQKPGQSPRLLIY |
| Clone | FR3 |
| AB1, AB3, AB6, AB5, | SEQ ID NO: 43 |
| AB2, AB11 | KRFSGVPDRFSGSGSGTDFTLKISRVEA |
| | EDLGVYYC |
| AB4 | SEQ ID NO: 44 |
| | KRFSGVPDRFSGSGSGTDFTLKISRVGA |
| | EDLGVYYC |
| AB7 | SEQ ID NO: 45 |
| | NRLSGVPDRFSGSGSGTDFTLKISRVEA |
| | EDLGVYFC |
| hAB6, 3-3 | SEQ ID NO: 86 |
| | KRFSGVPDRFSGSGSGTDFTLKISRVEA |
| | EDVGVYYC |
| Clone | FR4 |
| AB1, AB2, AB3, AB4, | SEQ ID NO: 46 |
| AB5, AB6, AB11 | FGAGTKLELKR |
| AB7 | SEQ ID NO: 47 |
| | FGGGTKLEIKR |
| hAB6, 3-3 | SEQ ID NO: 87 |
| | FGQGTKLEIKR |

In certain embodiments there is provided an antigen binding protein having a sequence shown in Table 5 or 6 below:

TABLE 5
V_{H} domain

| Clone | Antigen binding protein sequence |
|---|---|
| AB1 | SEQ ID NO: 48 |
| | EVNLVESGGGLVQPGGSLKLSCAASGFTFSDYYMYWVRQTPEK |
| | RLEWVTYITNGDGRTYYSDTVRGRFTISRDNAKNTLYLQMSRL |
| | KSEDTAMYYCTSPPLGGAWFGYWGQGTLVTVS |
| AB2 | SEQ ID NO: 49 |
| | EVNLVESGGGLVQPGGSLKLSCEASGFSFSDYYMYWVRQTPEK |
| | RLEWVTYITNGGGRTYYSDTIRGRFTISRDNARNTLYLQMSRL |
| | KSEDTAMYYCTSPPLGGAWFGYWGQGTLVTVS |
| AB3 | SEQ ID NO: 50 |
| | EVNLVESGGGLVQPGGSLKLSCAASGFTFSDYYLYWVRQTPEK |
| | RLEWVTYITNGGGRTYYSDTVRGRFTISRDNAKNTLYLQMSRL |
| | KSEDTAMYYCASPPLGGAWFGYWGQGTLVTVS |
| AB4 | SEQ ID NO: 51 |
| | EVNLVESGGGLVQPGGSLKLSCAASGFTFSDYYMYWVRQTPEK |
| | RLEWVTYITNGGGRTYYSDTVRGRFTISRDNAKNTLYLQMSRL |
| | KSEDTAMYYCASPPLGGAWFGYWGQGTLVTVS |
| AB5 | SEQ ID NO: 52 |
| | EVNLVESGGGLVQPGGSLKLSCEASGFTFSDYYMYWVRQTPEK |
| | RLEWVTYITNGAGRTYYSDTVRGRFTISRDNAKNTLYLQMSRL |
| | KSEDTAMYYCASPPLGGAWFAYWGQGTLVTVS |
| AB6 | SEQ ID NO: 53 |
| | EVKLVESGGGLVQPGGSLKLSCAASGFPFSDYYLYWVRQTPEK |
| | RLEWVAYITNGVGRTYYSDSVKGRFTISRDNAKNTLYLQMSRL |
| | KSEDTSMYYCTSPPLGGAWFAYWGQGTLVTVS |
| AB7 | SEQ ID NO: 54 |
| | QDQLQQSGVALARPGASVKLSCKASEYTFKSFGLGWVKQRPGQ |
| | GLEWIGEIYPRSGNTYYNEKVKGKVRLTADKSSNSVYMEFRSL |
| | TSEDSAVYFCARSPYDGYFDYWGQGTTLTVS |
| hAB6 | SEQ ID NO: 88 |
| | EVQLVESGGGLVQPGGSLRLSCAASGFPFSDYYLYWVRQAPGK |
| | GLEWVAYITNGVGRTYYSDSVKGRFTISRDNAKNTLYLQMNSL |
| | RDEDTAVYYCTSPPLGGAWFAYWGQGTLVTVS |
| AB11 | SEQ ID NO: 92 |
| | EVNLVESGGGLVQPGGSLILSCEASGFSFSDYYMYWVRQTPEK |
| | RLEWVTYITNGGGRTYYSDAIRGRFTISRDNARNTLYLQMSRL |
| | KSEDTAMYYCTSPPLGGAWFGYWGQGTLVTVS |
| 1-21 | SEQ ID NO: 96 |
| | EVQLVESGGGLVQPGGSLRLSCAASGFPFSDYYLYWVRQAPGK |
| | GLEWVAYITNGVGRTYYSDSVKGRFTISRDNAKNTLYLQMNSL |
| | RDEDTAVYYCTSPPLGGAWFGYWGQGTLVTVS |
| 1-23 | SEQ ID NO: 97 |
| | EVQLVESGGGLVQPGGSLRLSCVASGFPFSDYYLYWVRQAPGK |
| | GLEWVAYITNGVGRTYYSDSVKGRFTISRDNAKNTLYLQMNSL |
| | RDEDTAVYYCTSPPLGGAWFGYWGQGTLVTVS |

TABLE 6
V_{L} domain

| Clone | Antigen binding protein sequence |
|---|---|
| AB1 | SEQ ID NO: 55 |
| | DVLMTQTPLSLPVSLGDQASISCRSSQSIVHSNGNTYLEWYLQKPGQSP |
| | KLLIYKVSKRFSGVPDRFSGSGSGTDFTLKISRVEAEDLGVYYCFQGSH |
| | VPLTFGAGTKLELKR |
| AB2 | SEQ ID NO: 56 |
| | DVSMTQTPLSLPVSLGDQASISCRSSQSLVHSNGNTYLEWYLQKPGQS |
| | PKLLIYKVSKRFSGVPDRFSGSGSGTDFTLKISRVEAEDLGVYYCFQGS |
| | HVPLTFGAGTKLELKR |
| AB3 | SEQ ID NO: 57 |
| | DVLMTQTPLSLPVSLGDQASISCRSSQSIVHSNGNTYLEWYLQKPGQSP |
| | KLLIYKVSKRFSGVPDRFSGSGSGTDFTLKISRVEAEDLGVYYCFQGSH |
| | VPLTFGAGTKLELKR |
| AB4 | SEQ ID NO: 58 |
| | DVLMTQTPLSLPVSLGDQASISCRSSQSLVHSNGNTYLEWYLQKPGQS |
| | PKLLIYKVSKRFSGVPDRFSGSGSGTDFTLKISRVGAEDLGVYYCFQGS |
| | HVPLTFGAGTKLELKR |
| AB5 | SEQ ID NO: 56 |
| | DVSMTQTPLSLPVSLGDQASISCRSSQSLVHSNGNTYLEWYLQKPGQS |
| | PKLLIYKVSKRFSGVPDRFSGSGSGTDFTLKISRVEAEDLGVYYCFQGS |
| | HVPLTFGAGTKLELKR |
| AB6 | SEQ ID NO: 55 |
| | DVLMTQTPLSLPVSLGDQASISCRSSQSIVHSNGNTYLEWYLQKPGQSP |
| | KLLIYKVSKRFSGVPDRFSGSGSGTDFTLKISRVEAEDLGVYYCFQGSH |
| | VPLTFGAGTKLELKR |
| AB7 | SEQ ID NO: 59 |
| | DVVMTHSPLSLPVSLGDQASISCRSSQSLLHINGNTYLHWYLQKPGQSP |
| | KLLIYRVSNRLSGVPDRFSGSGSGTDFTLKISRVEAEDLGVYFCSQSTH |
| | VPRTFGGGTKLEIKR |
| hAB6 | SEQ ID NO: 89 |
| | DIVMTQSPLSLPVTPGEPASISCRSSQSIVHSNGNTYLEWYLQKPGQSP |
| | RLLIYKVSKRFSGVPDRFSGSGSGTDFTLKISRVEAEDVGVYYCFQGSH |
| | VPLTFGQGTKLEIKR |
| AB11 | SEQ ID NO: 93 |
| | DVSMTQTPLSLPVSLGDQASISCRSSQSLVHSNGNTYLEWYLQKPGQS |
| | PKLLIYKVSKRFSGVPDRFSGSGSGTDFTLKISRVEAEDLGVYYCFQGS |
| | HVPLTFGAGTKLELKR |
| 3-3 | SEQ ID NO: 98 |
| | DIVMTQSPLSLPVTPGEPASISCRSSRSIVHSNGNTYLEWYLQKPGQSP |
| | RLLIYKVSKRFSGVPDRFSGSGSGTDFTLKISRVEAEDVGVYYCFQGSH |
| | VPLTFGQGTKLEIKR |

TABLE 7
Fc regions of hIgG1, 3SFc and 3MFc

| Clone | Fc (bold is CH1, italics hinge region, underlined is CH2 and no formatting is CH3) |
|---|---|
| hIgG1 | SEQ ID NO: 60 |
| | A S T K G P S V F P L A P S S K S T S G G T A A L G C L V K D Y F P E |
| | P V T V S W N S G A L T S G V H T F P A V L Q S S G L Y S L S S V V T |
| | V P S S S L G T Q T Y I C N V N H K P S N T K V D K K V E P K S C D K |
| | T H T C P P C P A P E L L G G P S V F L F P P K P K D T L M I S R T P |
| | E V T C V V V D V S H E D P E V K F N W Y V D G V E V H N A K T K P R |
| | E E Q Y N S T Y R V V S V L T V L H Q D W L N G K E Y K C K V S N K A |
| | L P A P I E K T I S K A K G Q P R E P Q V Y T L P P S R D E L T K N Q |
| | V S L T C L V K G F Y P S D I A V E W E S N G Q P E N N Y K T T P P V |
| | L D S D G S F F L Y S K L T V D K S R W Q Q G N V F S C S V M H E A L |
| | H N H Y T Q K S L S L S P G K |
| 3SFc | SEQ ID NO: 61 |
| | A S T K G P S V F P L A P S S K S T S G G T A A L G C L V K D Y F P E |
| | P V T V S W N S G A L T S G V H T F P A V L Q S S G L Y S L S S V V T |
| | V P S S S L G T Q T Y I C N V N H K P S N T K V D K K V E P K S C D K |
| | T H T C P P C P A P E F E G G P S V F L F P P K P K D T L M I S R T P |
| | E V T C V V V D V S H E D P E V K F N W Y V D G V E V H N A K T K P R |
| | E E Q Y N S T Y R V V S V L T V L H Q D W L N G K E Y K C K V S N K A |
| | L P A S I E K T I S K A K G Q P R E P Q V Y T L P P S R D E L T K N Q |
| | V S L T C L V K G F Y P S D I A V E W E S N G Q P E N N Y K T T P P V |
| | L D S D G S F F L Y S K L T V D K S R W Q Q G N V F S C S V M H E A L |
| | H N H Y T Q K S L S L S P G K |
| 3MFc | SEQ ID NO: 62 |
| | A S T K G P S V F P L A P S S K S T S G G T A A L G C L V K D Y F P E |
| | P V T V S W N S G A L T S G V H T F P A V L Q S S G L Y S L S S V V T |
| | V P S S S L G T Q T Y I C N V N H K P S N T K V D K K V E P K S C D K |
| | T H T C P P C P A P E L L G G P D V F L F P P K P K D T L M I S R T P |
| | E V T C V V V D V S H E D P E V K F N W Y V D G V E V H N A K T K P R |
| | E E Q Y N S T Y R V V S V L T V L H Q D W L N G K E Y K C K V S N K A |
| | L P L P E E K T I S K A K G Q P R E P Q V Y T L P P S R D E L T K N Q |
| | V S L T C L V K G F Y P S D I A V E W E S N G Q P E N N Y K T T P P V |
| | L D S D G S F F L Y S K L T V D K S R W Q Q G N V F S C S V M H E A L |
| | H N H Y T Q K S L S L S P G K |

TABLE 8
Nucleic acid sequence encoding one or more of the amino acid
sequences of the invention

| Description | Nucleic acid sequence |
|---|---|
| AB6 VH | SEQ ID NO: 63 |
| domain | GAAGTGAAGTTGGTGGAGTCTGGGGGAGGCTTAGTGCAGCCTGGA |
| | GGGTCCCTGAAACTCTCCTGTGCAGCCTCTGGATTCCCTTTTAGTGA |
| | CTATTACCTGTATTGGGTTCGCCAGACTCCAGAGAAGAGGCTGGAG |
| | TGGGTCGCATACATCACTAATGGTGTTGGTAGGACCTATTATTCAGA |
| | CTCTGTAAAGGGCCGATTCACCATCTCCAGAGACAATGCCAAGAAC |
| | ACCCTGTACCTGCAAATGAGCCGTCTGAAGTCTGAGGACACATCCA |
| | TGTATTACTGTACTAGTCCCCCACTGGGGGGGGCCTGGTTTGCTTA |
| | CTGGGGCCAAGGGACTCTGGTCACTGTCTCT |
| AB1 VH | SEQ ID NO: 64 |
| domain | GAAGTGAACCTGGTGGAGTCTGGGGGAGGCTTAGTGCAGCCTGGA |
| | GGGTCCCTGAAACTCTCCTGTGCAGCCTCTGGATTCACTTTCAGTG |
| | ACTATTACATGTATTGGGTTCGCCAGACTCCAGAGAAGAGGCTGGA |
| | GTGGGTCACATATATTACTAATGGTGATGGTAGGACCTATTATTCAG |
| | ACACTGTAAGGGGCCGATTCACCATATCCAGAGACAATGCCAAGAA |
| | CACCCTGTACCTGCAAATGAGCCGTCTGAAGTCTGAGGACACAGCC |
| | ATGTATTACTGTACAAGTCCTCCACTGGGAGGGGCCTGGTTTGGTT |
| | ACTGGGGCCAAGGGACTCTGGTCACTGTCTCT |
| AB2 VH | SEQ ID NO: 65 |
| domain | GAAGTGAACCTGGTGGAGTCTGGGGGAGGCTTAGTGCAGCCTGGA |
| | GGGTCCCTGAAACTCTCCTGTGAAGCCTCTGGATTCAGTTTCAGTG |
| | ACTATTACATGTATTGGGTTCGCCAGACTCCAGAGAAGCGGCTGGA |
| | GTGGGTCACATATATTACTAATGGTGGTGGTAGAACCTATTACTCAG |
| | ACACTATAAGGGGCCGATTCACCATCTCCAGAGACAATGCCAGGAA |
| | CACCCTGTACCTGCAAATGAGCCGTCTGAAGTCTGAGGACACAGCC |
| | ATGTATTACTGTACAAGTCCCCCACTGGGGGGGGCCTGGTTTGGTT |
| | ACTGGGGCCAAGGGACTCTGGTCACTGTTTCT |
| AB3 VH | SEQ ID NO: 66 |
| domain | GAAGTGAACCTGGTGGAGTCTGGGGGAGGCTTAGTGCAGCCTGGAGGGTCCCTG |
| | AAACTCTCCTGTGCAGCCTCTGGATTCACTTTCAGTGACTATTATTTATATTGG |
| | GTTCGCCAGACTCCAGAGAAGAGGCTGGAGTGGGTCACATATATTACTAATGGT |
| | GGTGGTAGGACCTATTATTCAGACACTGTAAGGGGCCGATTCACCATATCCAGA |
| | GACAATGCCAAGAACACCCTGTACCTGCAAATGAGCCGTCTGAAGTCTGAGGAC |
| | ACAGCCATGTATTACTGTGCAAGTCCTCCACTGGGAGGGGCCTGGTTTGGTTAC |
| | TGGGGCCAAGGGACTCTGGTCACTGTCTCT |
| AB4 VH | SEQ ID NO: 67 |
| domain | GAAGTGAACCTGGTGGAGTCTGGGGGAGGCTTAGTGCAGCCTGGA |
| | GGGTCCCTGAAACTCTCCTGTGCAGCCTCTGGATTCACTTTCAGTG |
| | ACTATTACATGTATTGGGTTCGCCAGACTCCAGAGAAGAGGCTGGA |
| | GTGGGTCACATATATTACTAATGGTGGTGGTAGGACCTATTATTCAG |
| | ACACTGTAAGGGGCCGATTCACCATATCCAGAGACAATGCCAAGAA |
| | CACCCTGTACCTACAAATGAGCCGTCTGAAGTCTGAGGACACAGCC |
| | ATGTATTACTGTGCAAGTCCTCCACTGGGAGGGGCCTGGTTTGGTT |
| | ACTGGGGCCAAGGGACTCTGGTCACTGTCTCT |
| AB5 VH | SEQ ID NO: 68 |
| domain | GAAGTGAACCTGGTGGAGTCTGGGGGAGGCTTAGTGCAGCCTGGA |
| | GGGTCCCTGAAACTCTCCTGTGAAGCCTCTGGATTCACTTTCAGTGA |
| | CTATTACATGTATTGGGTTCGCCAGACTCCAGAGAAGAGGCTGGAG |
| | TGGGTCACATATATTACTAATGGTGCTGGTAGAACCTATTACTCAGA |
| | CACTGTAAGGGGCCGATTCACCATCTCCAGAGACAATGCCAAGAAC |
| | ACCCTGTACCTGCAAATGAGCCGTCTGAAGTCTGAGGACACAGCCA |
| | TGTATTACTGTGCAAGTCCCCCACTGGGAGGGGCCTGGTTTGCTTA |
| | CTGGGGCCAAGGGACTCTGGTCACTGTCTCT |
| AB7 VH | SEQ ID NO: 69 |
| domain | CAGGATCAGTTACAGCAGTCTGGAGTTGCGCTGGCGAGGCCTGGG |
| | GCTTCAGTGAAGCTGTCCTGCAAGGCTTCTGAATACACCTTCAAAAG |
| | CTTTGGTTTAGGCTGGGTGAAGCAGAGACCTGGTCAGGGCCTTGAG |
| | TGGATTGGAGAGATTTATCCTAGAAGTGGTAATACTTACTACAATGA |
| | GAAGGTCAAGGGCAAGGTCAGACTGACTGCAGACAAATCCTCCAAC |
| | TCAGTTTACATGGAGTTCCGCAGCCTGACATCTGAGGACTCTGCGG |
| | TCTATTTCTGTGCACGATCCCCCTATGATGGTTACTTTGACTACTGG |
| | GGCCAAGGCACCACTCTCACAGTCTCC |
| AB1 VL | SEQ ID NO: 70 |
| domain | GATGTTTTGATGACCCAAACTCCACTCTCCCTGCCTGTCAGTCTTGG |
| | AGATCAAGCCTCCATCTCTTGCAGATCTAGTCAGAGTATTGTACATA |
| | GTAATGGAAACACCTATTTAGAATGGTACCTGCAGAAACCAGGCCA |
| | GTCTCCAAAGCTCCTGATCTACAAAGTTTCCAAACGATTTTCTGGGG |
| | TCCCAGACAGGTTCAGTGGCAGTGGATCAGGGACAGATTTCACACT |
| | CAAGATCAGCAGAGTGGAGGCTGAAGATCTGGGAGTTTATTACTGT |
| | TTTCAAGGTTCACATGTTCCGCTCACGTTCGGTGCTGGGACCAAGC |
| | TGGAGCTGAAACGG |
| AB3 VL | SEQ ID NO: 71 |
| domain | GATGTTTTGATGACCCAAACTCCACTCTCCCTGCCTGTCAGTCTTGG |
| | AGATCAAGCCTCCATCTCTTGCAGATCTAGTCAGAGTATTGTACATA |
| | GTAATGGAAACACCTATTTAGAATGGTACCTGCAGAAACCAGGCCA |
| | GTCTCCAAAGCTCCTGATCTACAAAGTTTCCAAACGATTTTCTGGGG |
| | TCCCAGACAGGTTCAGTGGCAGTGGATCAGGGACAGATTTCACACT |
| | CAAGATCAGCAGAGTGGAGGCTGAGGATCTGGGAGTTTATTACTGT |
| | TTTCAAGGTTCACATGTTCCGCTCACGTTCGGTGCTGGGACCAAACT |
| | GGAGCTGAAACGG |
| AB2 VL | SEQ ID NO: 72 |
| domain | GATGTTTCGATGACCCAAACTCCACTCTCCCTGCCTGTCAGTCTTGG |
| | AGATCAAGCCTCCATCTCTTGCAGATCTAGTCAGAGCCTTGTACATA |
| | GTAATGGAAACACCTATTTAGAATGGTACCTGCAGAAACCAGGCCA |
| | GTCTCCAAAGCTCCTGATCTACAAAGTTTCCAAGCGATTTTCTGGGG |
| | TCCCAGACAGGTTCAGTGGCAGTGGATCAGGGACAGATTTCACACT |
| | CAAGATCAGCAGAGTGGAGGCTGAGGATCTGGGAGTTTATTACTGT |
| | TTTCAAGGTTCACATGTTCCGCTCACGTTCGGTGCTGGGACCAAGC |
| | TGGAGCTGAAACGG |
| AB6 VL | SEQ ID NO: 73 |
| domain | GATGTTTTGATGACCCAAACTCCACTCTCCCTGCCTGTCAGTCTTGG |
| | AGATCAAGCCTCCATCTCTTGCAGATCTAGTCAGAGTATTGTACATA |
| | GTAATGGAAACACCTATTTAGAATGGTACCTGCAGAAACCAGGCCA |
| | GTCTCCAAAGCTCCTGATCTACAAAGTTTCCAAACGATTTTCTGGGG |
| | TCCCAGACAGGTTCAGTGGCAGTGGATCAGGGACAGATTTCACACT |
| | CAAGATCAGCAGAGTGGAGGCTGAAGATCTGGGAGTTTATTACTGT |
| | TTTCAAGGTTCACATGTTCCGCTCACGTTCGGTGCTGGGACCAAGC |
| | TGGAGCTGAAACGG |
| AB4 VL | SEQ ID NO: 74 |
| domain | GATGTTTTGATGACCCAAACTCCACTCTCCCTGCCTGTCAGTCTTGG |
| | AGATCAAGCCTCCATCTCTTGCAGATCTAGTCAGAGTCTTGTACATA |
| | GTAATGGAAACACCTATTTAGAATGGTACCTGCAGAAACCAGGCCA |
| | GTCTCCAAAGCTCCTGATCTACAAAGTTTCCAAACGATTTTCTGGGG |
| | TCCCAGACAGGTTCAGTGGCAGTGGATCAGGGACAGATTTCACACT |
| | CAAGATCAGCAGAGTGGGGGCTGAGGATCTGGGAGTTTATTACTGT |
| | TTTCAAGGTTCACATGTTCCGCTCACGTTCGGTGCTGGGACCAAGC |
| | TGGAGCTGAAACGG |
| AB5 VL | SEQ ID NO: 75 |
| domain | GATGTTTCGATGACCCAAACTCCACTCTCCCTGCCTGTCAGTCTTGG |
| | AGATCAAGCCTCCATCTCTTGCAGATCTAGTCAGAGCCTTGTACATA |
| | GCAATGGAAACACCTATTTAGAATGGTACCTGCAGAAACCAGGCCA |
| | GTCTCCAAAGCTCCTGATCTACAAAGTTTCCAAACGATTTTCTGGGG |
| | TCCCAGACAGGTTCAGTGGCAGTGGTTCAGGGACAGATTTCACACT |
| | CAAGATCAGCAGAGTGGAGGCTGAGGATCTGGGAGTTTATTACTGT |
| | TTTCAAGGTTCACATGTTCCGCTCACGTTCGGTGCTGGGACCAAGC |
| | TGGAGCTGAAACGG |
| AB7 VL | SEQ ID NO: 76 |
| domain | GATGTTGTGATGACCCATTCTCCACTCTCCCTACCTGTCAGTCTTGG |
| | AGATCAGGCCTCCATCTCTTGCAGATCTAGTCAGAGCCTTCTGCACA |
| | TTAATGGAAACACCTATTTACATTGGTACCTGCAGAAGCCAGGCCAG |
| | TCTCCAAAGCTCCTGATCTACAGAGTTTCCAACCGATTATCTGGGGT |
| | CCCAGACAGGTTCAGTGGCAGTGGATCAGGGACAGATTTCACACTC |
| | AAGATCAGCAGAGTGGAGGCTGAGGATCTGGGAGTTTATTTCTGCT |
| | CTCAAAGTACACATGTTCCTCGGACGTTCGGTGGAGGCACCAAGCT |
| | GGAAATCAAACGG |
| hIgG1 | SEQ ID NO: 77 |
| | GCTAGCACCAAGGGCCCATCGGTCTTCCCCCTGGCACCCTCCTCCA |
| | AGAGCACCTCTGGGGGCACAGCGGCCCTGGGCTGCCTGGTCAAGG |
| | ACTACTTCCCCGAACCGGTGACGGTGTCGTGGAACTCAGGCGCCCT |
| | GACCAGCGGCGTGCACACCTTCCCGGCTGTCCTACAGTCCTCAGG |
| | ACTCTACTCCCTCAGCAGCGTGGTGACCGTGCCCTCCAGCAGCTTG |
| | GGCACCCAGACCTACATCTGCAACGTGAATCACAAGCCCAGCAACA |
| | CCAAGGTGGACAAGAAAGTTGAGCCCAAATCTTGTGACAAAACTCA |
| | CACATGCCCACCGTGCCCAGCACCTGAATTCGAGGGGGGACCGTC |
| | AGTCTTCCTCTTCCCCCCAAAACCCAAGGACACCCTCATGATCTCCC |
| | GGACCCCTGAGGTCACATGCGTGGTGGTGGACGTGAGCCACGAAG |
| | ACCCTGAGGTCAAGTTCAACTGGTACGTGGACGGCGTGGAGGTGC |
| | ATAATGCCAAGACAAAGCCGCGGGAGGAGCAGTACAACAGCACGTA |
| | CCGTGTGGTCAGCGTCCTCACCGTCCTGCACCAGGACTGGCTGAAT |
| | GGCAAGGAGTACAAGTGCAAGGTCTCCAACAAAGCCCTCCCAGCCA |
| | GCATCGAGAAAACCATCTCCAAAGCCAAAGGGCAGCCCCGAGAACC |
| | ACAGGTGTACACCCTGCCCCCATCCCGGGATGAGCTGACCAAGAAC |
| | CAGGTCAGCCTGACCTGCCTGGTCAAAGGCTTCTATCCCAGCGACA |
| | TCGCCGTGGAGTGGGAGAGCAATGGGCAGCCGGAGAACAACTACA |
| | AGACCACGCCTCCCGTGTTGGACTCCGACGGCTCCTTCTTCCTCTA |
| | CAGCAAGCTCACCGTGGACAAGAGCAGGTGGCAGCAGGGGAACGT |
| | CTTCTCATGCTCCGTGATGCATGAGGCTCTGCACAACCACTACACG |
| | CAGAAGAGTC TCTCCCTGTC TCCGGGTAAA |
| 3SFc | SEQ ID NO: 78 |
| | GCTAGCACCAAGGGCCCATCGGTCTTCCCCCTGGCACCCTCCTCCA |
| | AGAGCACCTCTGGGGGCACAGCGGCCCTGGGCTGCCTGGTCAAGG |
| | ACTACTTCCCCGAACCGGTGACGGTGTCGTGGAACTCAGGCGCCCT |
| | GACCAGCGGCGTGCACACCTTCCCGGCTGTCCTACAGTCCTCAGG |
| | ACTCTACTCCCTCAGCAGCGTGGTGACTGTGCCCTCCAGCAGCTTG |
| | GGCACCCAGACCTACATCTGCAACGTGAATCACAAGCCCAGCAACA |
| | CCAAGGTGGACAAGAAAGTTGAGCCCAAATCTTGTGACAAAACTCA |
| | CACATGCCCACCGTGCCCAGCACCTGAACTCCTGGGGGGACCGGA |
| | TGTCTTCCTCTTCCCCCCAAAACCCAAGGACACCCTCATGATCTCCC |
| | GGACCCCTGAGGTCACATGCGTGGTGGTGGACGTGAGCCACGAAG |
| | ACCCTGAGGTCAAGTTCAACTGGTACGTGGACGGCGTGGAGGTGC |
| | ATAATGCCAAGACAAAGCCGCGGGAGGAGCAGTACAACAGCACGTA |
| | CCGTGTGGTCAGCGTCCTCACCGTCCTGCACCAGGACTGGCTGAAT |
| | GGCAAGGAGTACAAGTGCAAGGTCTCCAACAAAGCCCTCCCATTGC |
| | CCGAGGAGAAAACCATCTCCAAAGCCAAAGGGCAGCCCCGAGAAC |
| | CACAGGTGTACACCCTGCCCCCATCCCGGGATGAGCTGACCAAGAA |
| | CCAGGTCAGCCTGACCTGCCTGGTCAAAGGCTTCTATCCCAGCGAC |
| | ATCGCCGTGGAGTGGGGAGAGCAATGGGCAGCCGGAGAACAACTA |
| | CAAGACCACGCCTCCCGTGTTGGACTCCGACGGCTCCTTCTTCCTC |
| | TACAGCAAGCTCACCGTGGACAAGAGCAGTGGCAGCAGGGGAACG |
| | TCTTCTCATGCTCCGTGATGCATGAGGCTCTGCACAACCACTACAC |
| | GCAGAAGAGTCTCTCCCTGTCTCCGGGTAAA |
| 3MFc | SEQ ID NO: 79 |
| | GCTAGCACCAAGGGCCCATCGGTCTTCCCCCTGGCACCCTCCTCCA |
| | AGAGCACCTCTGGGGGCACAGCGGCCCTGGGCTGCCTGGTCAAGG |
| | ACTACTTCCCCGAACCGGTGACGGTGTCGTGGAACTCAGGCGCCCT |
| | GACCAGCGGCGTGCACACCTTCCCGGCTGTCCTACAGTCCTCAGG |
| | ACTCTACTCCCTCAGCAGCGTGGTGACCGTGCCCTCCAGCAGCTTG |
| | GGCACCCAGACCTACATCTGCAACGTGAATCACAAGCCCAGCAACA |
| | CCAAGGTGGACAAGAAAGTTGAGCCCAAATCTTGTGACAAAACTCA |
| | CACATGCCCACCGTGCCCAGCACCTGAACTCCTGGGGGGACCGTC |
| | AGTCTTCCTCTTCCCCCCAAAACCCAAGGACACCCTCATGATCTCCC |
| | GGACCCCTGAGGTCACATGCGTGGTGGTGGACGTGAGCCACGAAG |
| | ACCCTGAGGTCAAGTTCAACTGGTACGTGGACGGCGTGGAGGTGC |
| | ATAATGCCAAGACAAAGCCGCGGGAGGAGCAGTACAACAGCACGTA |
| | CCGTGTGGTCAGCGTCCTCACCGTCCTGCACCAGGACTGGCTGAAT |
| | GGCAAGGAGTACAAGTGCAAGGTCTCCAACAAAGCCCTCCCAGCCC |
| | CCATCGAGAAAACCATCTCCAAAGCCAAAGGGCAGCCCCGAGAACC |
| | ACAGGTGTACACCCTGCCCCCATCCCGGGATGAGCTGACCAAGAAC |
| | CAGGTCAGCCTGACCTGCCTGGTCAAAGGCTTCTATCCCAGCGACA |
| | TCGCCGTGGAGTGGGAGAGCAATGGGCAGCCGGAGAACAACTACA |
| | AGACCACGCCTCCCGTGTTGGACTCCGACGGCTCCTTCTTCCTCTA |
| | CAGCAAGCTCACCGTGGACAAGAGCAGGTGGCAGCAGGGGAACGT |
| | CTTCTCATGCTCCGTGATGCATGAGGCTCTGCACAACCACTACACG |
| | CAGAAGAGTCTCTCCCTGTCTCCGGGTAAA |
| hAB6 VH | SEQ ID NO: 99 |
| domain | GAGGTGCAGCTGGTGGAATCCGGCGGAGGACTGGTGCAGCCTGGC |
| | GGCTCCCTGAGACTGTCTTGCGCCGCCTCCGGCTTCCCCTTCTCCG |
| | ACTACTACCTGTACTGGGTCCGACAGGCCCCAGGCAAGGGCCTGG |
| | AATGGGTGGCCTACATCACCAACGGCGTGGGCCGGACCTACTACTC |
| | CGACTCCGTGAAGGGCCGGTTCACCATCTCCCGGGACAACGCCAA |
| | GAACACCCTGTACCTGCAGATGAACTCCCTGCGGGACGAGGACAC |
| | CGCCGTGTACTACTGCACCTCCCCACCCCTGGGCGGAGCTTGGTTT |
| | GCTTACTGGGGCCAGGGCACCCTGGTCACCGTGTCC |
| hAB6 VL | SEQ ID NO: 100 |
| domain | GACATCGTGATGACCCAGTCCCCCCTGTCCCTGCCTGTGACACCTG |
| | GCGAGCCCGCCTCCATCTCCTGCCGGTCCTCCCAGTCCATCGTGCA |
| | CTCCAACGGCAACACCTACCTGGAATGGTATCTGCAGAAGCCCGGC |
| | CAGTCCCCTCGGCTGCTGATCTACAAGGTGTCCAAGCGGTTCTCCG |
| | GCGTGCCCGACAGATTCTCCGGCTCTGGCTCCGGCACCGACTTCA |
| | CCCTGAAGATCTCCCGGGTGGAAGCCGAGGACGTGGGCGTGTACT |
| | ACTGTTTTCAAGGCTCCCACGTGCCCCTGACCTTCGGCCAGGGCAC |
| | CAAGCTGGAAATCAAG |

In certain embodiments, the antigen binding proteins bind to an epitope of a CCR6, wherein the epitope includes amino acids 1 to 28 of CCR6. Preferably, the CCR6 is human. Typically, the epitope includes the amino acid sequence of 1 to 28 of SEQ ID NO: 1.

Mutations to Proteins

The present invention also provides an antigen binding protein or a nucleic acid encoding same having at least 80% identity to a sequence disclosed herein. In one example, an antigen binding protein or nucleic acid of the invention comprises sequence at least about 85% or 90% or 95% or 97% or 98% or 99% identical to a sequence disclosed herein.

Alternatively, or additionally, the antigen binding protein comprises a CDR (e.g., three CDRs) at least about 80% or 85% or 90% or 95% or 97% or 98% or 99% identical to CDR(s) of a VH or VL as described herein according to any example.

In another example, a nucleic acid of the invention comprises a sequence at least about 80% or 85% or 90% or 95% or 97% or 98% or 99% identical to a sequence encoding an antigen binding protein having a function as described herein according to any example. The present invention also encompasses nucleic acids encoding an antigen binding protein of the invention, which differs from a sequence exemplified herein as a result of degeneracy of the genetic code.

The % identity of a nucleic acid or polypeptide is determined by GAP (Needleman and Wunsch. Mol. Biol. 48, 443-453, 1970) analysis (GCG program) with a gap creation penalty=5, and a gap extension penalty=0.3. The query sequence is at least 50 residues in length, and the GAP analysis aligns the two sequences over a region of at least 50 residues. For example, the query sequence is at least 100 residues in length and the GAP analysis aligns the two sequences over a region of at least 100 residues. For example, the two sequences are aligned over their entire length.

The present invention also contemplates a nucleic acid that hybridizes under stringent hybridization conditions to a nucleic acid encoding an antigen binding protein described herein. A “moderate stringency” is defined herein as being a hybridization and/or washing carried out in 2×SSC buffer, 0.1% (w/v) SDS at a temperature in the range 45° C. to 65° C., or equivalent conditions. A “high stringency” is defined herein as being a hybridization and/or wash carried out in 0.1×SSC buffer, 0.1% (w/v) SDS, or lower salt concentration, and at a temperature of at least 65° C., or equivalent conditions. Reference herein to a particular level of stringency encompasses equivalent conditions using wash/hybridization solutions other than SSC known to those skilled in the art. For example, methods for calculating the temperature at which the strands of a double stranded nucleic acid will dissociate (also known as melting temperature, or Tm) are known in the art. A temperature that is similar to (e.g., within 5° C. or within 10° C.) or equal to the Tm of a nucleic acid is considered to be high stringency. Medium stringency is to be considered to be within 10° C. to 20° C. or 10° C. to 15° C. of the calculated Tm of the nucleic acid.

The present invention also contemplates mutant forms of an antigen binding protein of the invention comprising one or more conservative amino acid substitutions compared to a sequence set forth herein. In some examples, the antigen binding protein comprises 10 or fewer, e.g., 9 or 8 or 7 or 6 or 5 or 4 or 3 or 2 or 1 conservative amino acid substitutions. A “conservative amino acid substitution” is one in which the amino acid residue is replaced with an amino acid residue having a similar side chain and/or hydropathicity and/or hydrophilicity.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine). Hydropathic indices are described, for example in Kyte and Doolittle J. Mol. Biol., 157: 105-132, 1982 and hydrophylic indices are described in, e.g., U.S. Pat. No. 4,554,101.

The present invention also contemplates non-conservative amino acid changes. For example, of particular interest are substitutions of charged amino acids with another charged amino acid and with neutral or positively charged amino acids. In some examples, the antigen binding protein comprises 10 or fewer, e.g., 9 or 8 or 7 or 6 or 5 or 4 or 3 or 2 or 1 non-conservative amino acid substitutions.

In one example, the mutation(s) occur within a FR of an antigen binding domain of an antigen binding protein of the invention. In another example, the mutation(s) occur within a CDR of an antigen binding protein of the invention.

Exemplary methods for producing mutant forms of an antigen binding protein include:
mutagenesis of DNA (Thie et al., Methods Mol. Biol. 525: 309-322, 2009) or RNA (Kopsidas et al., Immunol. Lett. 107:163-168, 2006; Kopsidas et al. BMC Biotechnology, 7: 18, 2007; and WO1999/058661);
introducing a nucleic acid encoding the polypeptide into a mutator cell, e.g., XL-1Red, XL-mutS and XL-mutS-Kanr bacterial cells (Stratagene);
DNA shuffling, e.g., as disclosed in Stemmer, Nature 370: 389-91, 1994; and
site directed mutagenesis, e.g., as described in Dieffenbach (ed) and Dveksler (ed) (In: PCR Primer: A Laboratory Manual, Cold Spring Harbor Laboratories, N Y, 1995).

Exemplary methods for determining biological activity of the mutant antigen binding proteins of the invention will be apparent to the skilled artisan and/or described herein, e.g., antigen binding. For example, methods for determining antigen binding, competitive inhibition of binding, affinity, association, dissociation and therapeutic efficacy are described herein.

As used herein, the properties of amino acids are defined in the following table:

| | 3- | 1- | |
|---|---|---|---|
| | letter | letter | |
| Amino Acid | code | code | Properties |
| Alanine | Ala | A | aliphatic hydrophobic neutral |
| Arginine | Arg | R | polar hydrophilic charged (+) |
| Asparagine | Asn | N | polar hydrophilic neutral |
| Aspartate | Asp | D | polar hydrophilic charged (−) |
| Cysteine | Cys | C | polar hydrophobic neutral |
| Glutamine | Gln | Q | polar hydrophilic neutral |
| Glutamate | Glu | E | polar hydrophilic charged (−) |
| Glycine | Gly | G | aliphatic neutral |
| Histidine | His | H | aromatic polar hydrophilic charged (+) |
| Isoleucine | Ile | I | aliphatic hydrophobic neutral |
| Leucine | Leu | L | aliphatic hydrophobic neutral |
| Lysine | Lys | K | polar hydrophilic charged (+) |
| Methionine | Met | M | hydrophobic neutral |
| Phenylalanine | Phe | F | aromatic hydrophobic neutral |
| Proline | Pro | P | hydrophobic neutral |
| Serine | Ser | S | polar hydrophilic neutral |
| Threonine | Thr | T | polar hydrophilic neutral |
| Tryptophan | Trp | W | aromatic hydrophobic neutral |
| Tyrosine | Tyr | Y | aromatic polar hydrophobic |
| Valine | Val | V | aliphatic hydrophobic neutral |

Constant Regions

The present invention encompasses antigen binding proteins and/or antibodies described herein comprising a constant region of an antibody. This includes antigen binding fragments of an antibody fused to an Fc.

Sequences of constant regions useful for producing the proteins of the present invention may be obtained from a number of different sources. In some examples, the constant region or portion thereof of the protein is derived from a human antibody. The constant region or portion thereof may be derived from any antibody class, including IgM, IgG, IgD, IgA and IgE, and any antibody isotype, including IgG1, IgG2, IgG3 and IgG4. In one example, the constant region is human isotype IgG4 or a stabilized IgG4 constant region.

In one example, the Fc region of the constant region has a reduced ability to induce effector function, e.g., compared to a native or wild-type human IgG1 or IgG3 Fc region. In one example, the effector function is antibody-dependent cell-mediated cytotoxicity (ADCC) and/or antibody-dependent cell-mediated phagocytosis (ADCP) and/or complement-dependent cytotoxicity (CDC). Methods for assessing the level of effector function of an Fc region containing protein are known in the art and/or described herein.

In one example, the Fc region is an IgG4 Fc region (i.e., from an IgG4 constant region), e.g., a human IgG4 Fc region. Sequences of suitable IgG4 Fc regions will be apparent to the skilled person and/or available in publically available databases (e.g., available from National Center for Biotechnology Information).

In one example, the constant region is a stabilized IgG4 constant region. The term “stabilized IgG4 constant region” will be understood to mean an IgG4 constant region that has been modified to reduce Fab arm exchange or the propensity to undergo Fab arm exchange or formation of a half-antibody or a propensity to form a half antibody. “Fab arm exchange” refers to a type of protein modification for human IgG4, in which an IgG4 heavy chain and attached light chain (half-molecule) is swapped for a heavy-light chain pair from another IgG4 molecule. Thus, IgG4 molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half antibody” forms when an IgG4 antibody dissociates to form two molecules each containing a single heavy chain and a single light chain.

In one example, a stabilized IgG4 constant region comprises a proline at position 241 of the hinge region according to the system of Kabat (Kabat et al., Sequences of Proteins of Immunological Interest Washington DC United States Department of Health and Human Services, 1987 and/or 1991). This position corresponds to position 228 of the hinge region according to the EU numbering system (Kabat et al., Sequences of Proteins of Immunological Interest Washington DC United States Department of Health and Human Services, 2001 and Edelman et al., Proc. Natl. Acad. USA, 63, 78-85, 1969). In human IgG4, this residue is generally a serine. Following substitution of the serine for proline, the IgG4 hinge region comprises a sequence CPPC. In this regard, the skilled person will be aware that the “hinge region” is a proline-rich portion of an antibody heavy chain constant region that links the Fc and Fab regions that confers mobility on the two Fab arms of an antibody. The hinge region includes cysteine residues which are involved in inter-heavy chain disulfide bonds. It is generally defined as stretching from Glu226 to Pro243 of human IgG1 according to the numbering system of Kabat. Hinge regions of other IgG isotypes may be aligned with the IgG1 sequence by placing the first and last cysteine residues forming inter-heavy chain disulphide (S—S) bonds in the same positions (see for example WO2010/080538).

Additional examples of stabilized IgG4 antibodies are antibodies in which arginine at position 409 in a heavy chain constant region of human IgG4 (according to the EU numbering system) is substituted with lysine, threonine, methionine, or leucine (e.g., as described in WO2006/033386). The Fc region of the constant region may additionally or alternatively comprise a residue selected from the group consisting of: alanine, valine, glycine, isoleucine and leucine at the position corresponding to 405 (according to the EU numbering system). Optionally, the hinge region comprises a proline at position 241 (i.e., a CPPC sequence) (as described above).

In another example, the Fc region is a region modified to have reduced effector function, i.e., a “non-immunostimulatory Fc region”. For example, the Fc region is an IgG1 Fc region comprising a substitution at one or more positions selected from the group consisting of 268, 309, 330 and 331. In another example, the Fc region is an IgG1 Fc region comprising one or more of the following changes E233P, L234V, L235A and deletion of G236 and/or one or more of the following changes A327G, A330S and P331S (Armour et al., Eur J Immunol. 29:2613-2624, 1999; Shields et al., J Biol Chem. 276(9):6591-604, 2001). Additional examples of non-immunostimulatory Fc regions are described, for example, in Dall'Acqua et al., J Immunol. 177: 1129-1138 2006; and/or Hezareh J Virol; 75: 12161-12168, 2001).

In another example, the Fc region is a chimeric Fc region, e.g., comprising at least one C_{H}2 domain from an IgG4 antibody and at least one C_{H}3 domain from an IgG1 antibody, wherein the Fc region comprises a substitution at one or more amino acid positions selected from the group consisting of 240, 262, 264, 266, 297, 299, 307, 309, 323, 399, 409 and 427 (EU numbering) (e.g., as described in WO2010/085682). Exemplary substitutions include 240F, 262L, 264T, 266F, 297Q, 299A, 299K, 307P, 309K, 309M, 309P, 323F, 399S, and 427F.

As used herein an amino acid residue at the position equivalent to, for example, position 234, 235 or 331 in SEQ ID NO: 60 can be determined by any means known to a person skilled in the art. For example, an alignment of one or more sequences with an amino acid sequence of SEQ ID NO: 60 would allow a person skilled in the art to determine the amino acid at the position equivalent to position 234, 235 or 331 in SEQ ID NO: 60. A person skilled in the art can compare the three dimensional structure of a protein with the three dimensional structure of a protein having the amino acid sequence of SEQ ID NO: 60 and determine the amino acid residue that is at an equivalent position to position 234, 235 or 331 in SEQ ID NO: 60.

Antibody Binding Domain Containing Proteins

In another embodiment there is provided an antigen binding protein as described above wherein an amino acid sequence forming one or more of FR1, CDR1, FR2, CDR2, FR3, CDR3 and FR4 is derived from a human sequence or in the form of a human sequence.

The antigen binding protein may be presented in a humanized form including non-human (e.g., murine) and human immunoglobulin sequences. Typically all but the CDR sequences of the antigen binding protein are from a non-human species such as mouse, rat or rabbit. In some instances, framework residues of the antigen binding protein may also be non-human. Where the antigen binding protein is provided in the form of a whole antibody, typically at least a portion of an immunoglobulin constant region (Fc) is human, thereby allowing various human effector functions.

Methods for humanizing non-human antigen binding proteins are well known in the art, examples of suitable processes including those in Jones et al., (1986) Nature, 321:522; Riechmann et al., (1988) Nature, 332:323; Verhoeyen et al., (1988) Science, 239:1534.

Phage display methods described herein using antibody libraries derived from human immunoglobulin sequences are useful for generating human antigen binding proteins and human antibodies.

Also, transgenic mammals that are incapable of expressing functional endogenous immunoglobulins, but which can express human immunoglobulin genes can be used. These mice may be generated by random or targeted insertion of the human heavy and light chain immunoglobulin genes into embryonic stem cells. The host heavy and light chain immunoglobulin genes may be rendered non-functional by the insertion or by some other recombination event, for example by homozygous deletion of the host JH region. The transfected embryonic stem cells are expanded and microinjected into blastocysts to produce chimeric mice that are then bred to produce homozygous offspring that express human antigen binding proteins. After immunization with a CCR6 epitope, human monoclonal antibodies can be obtained. One benefit of transgenic animal systems is that it is possible to produce therapeutically useful isotypes because the human immunoglobulin transgenes rearrange during B-cell differentiation and subsequently undergo class switching and somatic mutation in the transgenic mice.

Variable domains including CDRs and FRs of the invention may have been made less immunogenic by replacing surface-exposed residues so as to make the antibody appear as self to the immune system. Padlan, E. A., 1991, Mol. Immunol. 28, 489 provides an exemplary method. Generally, affinity is preserved because the internal packing of amino acid residues in the vicinity of the antigen binding protein remains unchanged and generally CDR residues or adjacent residues which influence binding characteristics are not to be substituted in these processes.

In another embodiment there is provided an anti-CCR6 antigen binding protein, immunoglobulin variable domain, antibody, dab, scFv, Fab, Fab′, F(ab′)2, Fv fragment, diabody, triabody, linear antibody, single-chain antibody molecule, or multispecific antibody as described herein, preferably with a sequence as shown in any one of Tables 1 to 6.

Lower molecular weight antibody fragments, as compared with whole antibodies may have improved access to solid tumors and more rapid clearance which may be particularly useful in therapeutic and in vivo diagnostic applications.

In certain embodiments, the antigen binding protein is provided in the form of a single chain Fv fragment (scFv). Fv and scFv are suitable for reduced nonspecific binding during in vivo use as they have intact combining sites that are devoid of constant regions. Fusion proteins including scFv may be constructed to yield fusion of an effector protein at either the amino or the carboxy terminus of an scFv.

In another embodiment there is provided a diabody or triabody or other multispecific antibody including an antigen binding protein as described above. Multispecific antibodies may be assembled using polypeptide domains that allow for multimerization. Examples include the CH2 and CH3 regions of the Fc and the CH1 and Ckappa/lambda regions. Other naturally occurring protein multimerization domains may be used including leucine zipper domain (bZIP), helix-loop-helix motif, Src homology domain (SH2, SH3), an EF hand, a phosphotyrosine binding (PTB) domain, or other domains known in the art.

In another embodiment there is provided a fusion domain or heterologous protein including an antigen binding protein, immunoglobulin variable domain, antibody, dab, scFv, Fab, Fab′, F(ab′)2, Fv fragment, diabody, triabody, linear antibody, single-chain antibody molecule, or multispecific antibody as described herein.

A heterologous polypeptide may be recombinantly fused or chemically conjugated to an N- or C-terminus of an antigen binding protein or molecule containing same of the invention.

The heterologous polypeptide to which the antibody or antigen binding protein is fused may be useful to target to the CCR6 expressing cells, or useful to some other function such as purification, or increasing the in vivo half-life of the polypeptides, or for use in immunoassays using methods known in the art.

In preferred embodiments, a marker amino acid sequence such as a hexa-histidine peptide is useful for convenient purification of the fusion protein. Others include, but are not limited to, the “HA” tag, which corresponds to an epitope derived from the influenza hemagglutinin protein and the “flag” tag.

Further, the antigen binding protein, immunoglobulin variable domain, antibody, dab, scFv, Fab, Fab′, F(ab′)2, Fv fragment, diabody, triabody, linear antibody, single-chain antibody molecule, or multispecific antibody of the invention may be modified by glycosylation, acetylation, pegylation, phosphorylation, amidation, derivatization by known protecting/blocking groups, proteolytic cleavage, linkage to a cellular ligand or other protein, etc.

Antigen binding proteins of the invention can be composed of amino acids joined to each other by peptide bonds or modified peptide bonds, i.e., peptide isosteres, and may contain amino acids other than the 20 gene-encoded amino acids. Antigen binding proteins of the invention may be modified by natural processes, such as posttranslational processing, or by chemical modification techniques which are well known in the art. Such modifications are well described in basic texts, as well as in research literature. Modifications can occur anywhere in the antigen binding protein, including the peptide backbone, the amino acid side-chains and the amino or carboxyl termini, or on moieties such as carbohydrates. It will be appreciated that the same type of modification may be present in the same or varying degrees at several proteins in a given antigen binding protein. Also, a given antigen binding protein may contain many types of modifications. An antigen binding protein may be branched, for example, as a result of ubiquitination, and they may be cyclic, with or without branching. Cyclic, branched, and branched cyclic antigen binding proteins may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination.

In another embodiment there is provided a conjugate in the form of an antigen binding protein, immunoglobulin variable domain, antibody, Fab, dab, scFv, diabody, triabody or fusion protein as described above conjugated to a cytotoxic agent such as a chemo therapeutic agent, a drug, a growth inhibitory agent, a toxin (e.g., an enzymatically active toxin of bacterial, fungal, plant, or animal origin, or fragments thereof), or a label such as a radioactive isotope (i.e., a radio conjugate). In another aspect, the invention further provides methods of using the immunoconjugates. In one aspect, an immunoconjugate comprises any of the above variable domains covalently attached to a cytotoxic agent or a detectable agent.

In another embodiment there is provided an antibody for binding to an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein or conjugate as described above.

In another embodiment there is provided a nucleic acid encoding an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein or conjugate as described above.

A polynucleotide encoding an CDR or FR according to any one of the general formulae described above, or an antigen binding protein comprised of same, may be generated from a nucleic acid from any source, for example by chemical synthesis or isolation from a cDNA or genomic library. For example a cDNA library may be generated from an antibody producing cell such as a B cell, plasma cell or hybridoma cell and the relevant nucleic acid isolated by PCR amplification using oligonucleotides directed to the particular clone of interest. Isolated nucleic acids may then be cloned into vectors using any method known in the art. The relevant nucleotide sequence may then be mutagenized using methods known in the art e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N. Y. and Ausubel et al., eds., 1998, Current Protocols in Molecular Biology, John Wiley & Sons, NY), to generate antigen binding proteins having a different amino acid sequence, for example to create amino acid substitutions, deletions, and/or insertions.

Protein Production

In another embodiment there is provided a method for producing an anti CCR6 antigen binding protein as described above including expressing a nucleic acid as described above in a cell or non-human animal as described above.

The production of an antigen binding protein of the invention generally requires an expression vector containing a polynucleotide that encodes the antigen binding protein of the invention. A polynucleotide encoding an antigen binding protein of the invention may be obtained and sub cloned into a vector for the production of an antigen binding protein by recombinant DNA technology using techniques well-known in the art, including techniques described herein. Many different expression systems are contemplated including the use of mammalian cells including human cells for production and secretion of antigen binding proteins. Examples of cells include 293F, CHO and the NSO cell line.

Expression vectors containing protein coding sequences and appropriate transcriptional and translational control signals can be constructed using methods known in the art. These include in vitro recombinant DNA techniques, synthetic techniques and in vivo genetic recombination. In certain embodiments there is provided a replicable vector having a nucleic acid encoding an antigen binding protein operably linked to a promoter.

Cells transfected with an expression vector may be cultured by conventional techniques to produce an antigen binding protein. Thus, in certain embodiments, there is provided host cells or cell transfectants containing a polynucleotide encoding an antigen binding protein of the invention operably linked to a promoter. The promoter may be heterologous. A variety of host-expression vector systems may be utilized and in certain systems the transcription machinery of the vector system is particularly matched to the host cell. For example, mammalian cells such as Chinese hamster ovary cells (CHO) may be transfected with a vector including the major intermediate early gene promoter element from human cytomegalovirus. Additionally or alternatively, a host cell may be used that modulates the expression of inserted sequences, or modifies and processes the gene product as required, including various forms of post translational modification. Examples of mammalian host cells having particular post translation modification processes include CHO, VERY, BHK, Hela, COS, MDCK, 293, 3T3, W138, BT483, Hs578T, HTB2, BT2O and T47D, NSO, CRL7O3O and HsS78Bst cells.

Depending upon the use intended for the protein molecule, a number of bacterial expression vectors may be advantageously selected. In one example, vectors that cause the expression of high levels of fusion protein products that are readily purified, such as the E. coli expression vector pUR278 may be used where a large quantity of an antigen binding protein is to be produced. The expression product may be produced in the form of a fusion protein with lacZ. Other bacterial vectors include pIN vectors and the like. pGEX vectors may also be used to express foreign polypeptides as fusion proteins with glutathione-S-transferase (GST). These fusion proteins are generally soluble and can easily be purified from lysed cells by adsorption and binding to glutathione-agarose affinity matrix followed by elution in the presence of free glutathione. A thrombin and/or factor Xa protease cleavage site may be provided in the expressed polypeptide so that the cloned target gene product can be released from the GST moiety.

Autographa californica nuclear polyhedrosis virus (AcNPV) may be used as a vector to express foreign genes in an insect system including Spodoptera frugiperda cells. The particular promoter used may depend on where the protein coding is inserted into the sequence. For example, the sequence may be cloned individually into the polyhedrin gene and placed under control of the polyhedrin promoter.

Virus based expression systems may be utilized with mammalian cells such as an adenovirus whereby the coding sequence of interest may be ligated to the adenoviral late promoter and tripartite leader sequence. In vitro or in vivo recombination may then be used to insert this chimeric gene into the adenoviral genome. Insertions into region E1 or E3 will result in a viable recombinant virus that is capable of expressing the antigen binding protein in infected host cells. Specific initiation signals including the ATG initiation codon and adjacent sequences may be required for efficient translation of inserted antigen binding protein coding sequences. Initiation and translational control signals and codons can be obtained from a variety of origins, both natural and synthetic. Transcription enhancer elements and transcription terminators may be used to enhance the efficiency of expression of a viral based system.

Where long-term, high-yield production of recombinant proteins is required, stable expression is preferred. Generally a selectable marker gene is used whereby following transfection, cells are grown for 1-2 days in an enriched media and then transferred to a medium containing a selective medium in which cells containing the corresponding selectable marker, for example, antibiotic resistance can be screened. The result is that cells that have stably integrated the plasmid into their chromosomes grow and form foci that in turn can be cloned and expanded into cell lines. The herpes simplex virus thymidine kinase, hypoxanthineguanine phosphoribosyltransferase and adenine phosphoribosyltransferase genes are examples of genes that can be employed in tk-, hgprt- or aprT-cells, respectively, thereby providing appropriate selection systems. The following genes: dhfr, which confers resistance to methotrexate; gpt, which confers resistance to mycophenolic acid; neo, which confers resistance to the aminoglycoside G-418; and hygro, which confers resistance to hygromycin are examples of genes that can be used in anti-metabolite selection systems.

An antigen binding protein of the invention may be purified by a recombinant expression system by known methods including ion exchange chromatography, affinity chromatography (especially affinity for the specific antigens Protein A or Protein G) and gel filtration column chromatography), centrifugation, differential solubility, or by any other standard technique for the purification of proteins. Purification may be facilitated or assisted by providing the antigen binding protein in the form of a fusion protein.

Large quantities of the antigen binding proteins of the invention may be produced by a scalable process starting with a pilot expression system in a research laboratory that is scaled up to an analytical scale bioreactor (typically from 5 L to about 50 L bioreactors) or production scale bioreactors (for example, but not limited to 75 L, 100 L, 150 L, 300 L, or 500 L). Desirable scalable processes include those wherein there are low to undetectable levels of aggregation as measured by HPSEC or rCGE, typically no more than 5% aggregation by weight of protein down to no more than 0.5% by weight aggregation of protein. Additionally or alternatively, undetectable levels of fragmentation measured in terms of the total peak area representing the intact antigen binding protein may be desired in a scalable process so that at least 80% and as much as 99.5% or higher of the total peak area represents intact antigen binding protein. In other embodiments, the scalable process of the invention produces antigen binding proteins at production efficiency of about from 10 mg/L to about 300 mg/L or higher.

Various techniques have been developed for the production of antibody fragments including proteolytic digestion of intact antibodies and recombinant expression in host cells. With regard to the latter, as described below, Fab, Fv and scFv antibody fragments can all be expressed in and secreted from E. coli, antibody fragments can be isolated from the antibody phage libraries and Fab′-SH fragments can be directly recovered from E. coli and chemically coupled to form F(ab′)2 fragments. In another approach, F(ab′)2 fragments are isolated directly from recombinant host cell culture.

In another embodiment there is provided a vector including a nucleic acid described above. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The antigen binding site may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the antigen binding site-encoding DNA that is inserted into the vector. The signal sequence may be a prokaryotic signal sequence selected, for example, from the group of the alkaline phosphatase, penicillinase, Ipp, or heat-stable enterotoxin II leaders. For yeast secretion the signal sequence may be, e.g., the yeast invertase leader, alpha factor leader, or acid phosphatase leader or the C. albicans glucoamylase leader. In mammalian cell expression, mammalian signal sequences may be used to direct secretion of the protein, such as signal sequences from secreted polypeptides of the same or related species, as well as viral secretory leaders.

Polynucleotide sequences encoding polypeptide components of the antigen binding protein of the invention can be obtained using standard recombinant techniques as described above. Polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present invention. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides.

In general, plasmid vectors containing replicon and control sequences which are derived from species compatible with the host cell are used in connection with these hosts. Both expression and cloning vectors contain a nucleic acid sequence that enables the vector to replicate in one or more selected host cells, as well as marking sequences which are capable of providing phenotypic selection in transformed cells. Such sequences are well known for a variety of bacteria, yeast, and viruses. The origin of replication from the plasmid pBR322, which contains genes encoding ampicillin (Amp) and tetracycline (Tet) resistance and thus provides easy means for identifying transformed cells, is suitable for most Gram-negative bacteria, the 2 μm plasmid origin is suitable for yeast, and various viral origins (SV40, polyoma, adenovirus, VSV or BPV) are useful for cloning vectors in mammalian cells. pBR322, its derivatives, or other microbial plasmids or bacteriophage may also contain, or be modified to contain, promoters which can be used by the microbial organism for expression of endogenous proteins.

In addition, phage vectors containing replicon and control sequences that are compatible with the host microorganism can be used as transforming vectors in connection with these hosts. For example, bacteriophage such as λGEM.TM.-11 may be utilized in making a recombinant vector which can be used to transform susceptible host cells such as E. coli LE392.

The expression vector of the invention may comprise two or more promoter-cistron (a cistron being segment of DNA that contains all the information for production of single polypeptide) pairs. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the light or heavy chain by removing the promoter from the source DNA via restriction enzyme digestion and inserting the isolated promoter sequence into the vector of the invention. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters recognized by a variety of potential host cells are well known. Promoters suitable for use with prokaryotic hosts include the PhoA promoter, the β-galactamase and lactose promoter systems, alkaline phosphatase, a tryptophan (trp) promoter system and hybrid promoters such as the tac or the trc promoter. Promoters for use in bacterial systems also will contain a Shine-Dalgarno (S.D.) sequence operably linked to the DNA encoding an antigen binding protein of the invention. However, other promoters that are functional in bacteria (such as other known bacterial or phage promoters) are suitable as well. Their nucleotide sequences have been published, thereby enabling a skilled person operably to ligate them to cistrons encoding the target light and heavy chains using linkers or adaptors to supply any required restriction sites.

In one aspect of the invention, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PeIB, OmpA and MBP. In one embodiment of the invention, the signal sequences used in both cistrons of the expression system are STII signal sequences or variants thereof.

In another aspect, the production of the immunoglobulins according to the invention can occur in the cytoplasm of the host cell, and therefore does not require the presence of secretion signal sequences within each cistron. In that regard, immunoglobulin light and heavy chains are expressed, folded and assembled to form functional immunoglobulins within the cytoplasm. Certain host strains (e.g., the E. coli trxB strains) provide cytoplasm conditions that are favourable for disulfide bond formation, thereby permitting proper folding and assembly of expressed protein subunits.

The present invention provides an expression system in which the quantitative ratio of expressed polypeptide components can be modulated in order to maximize the yield of secreted and properly assembled antigen binding proteins of the invention. Such modulation is accomplished at least in part by simultaneously modulating translational strengths for the polypeptide components.

In terms of expression in eukaryotic host cells, the vector components generally include, but are not limited to, one or more of the following: a signal sequence, an origin of replication, one or more marker genes, an enhancer element, a promoter, and a transcription termination sequence.

A vector for use in a eukaryotic host cell may also contain a signal sequence or other polypeptide having a specific cleavage site at the N-terminus of the mature protein or polypeptide of interest. The heterologous signal sequence selected preferably is one that is recognized and processed {i.e., cleaved by a signal peptidase) by the host cell. In mammalian cell expression, mammalian signal sequences as well as viral secretory leaders, for example, the herpes simplex gD signal, are available.

The DNA for such precursor region is ligated in reading frame to DNA encoding the antibody.

Generally, an origin of replication component is not needed for mammalian expression vectors. For example, the SV40 origin may typically be used only because it contains the early promoter.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

An example of suitable selectable markers for mammalian cells are those that enable the identification of cells competent to take up the antigen binding protein-encoding nucleic acid, such as DHFR or thymidine kinase, metallothionein-I and -II, preferably primate metallothionein genes, adenosine deaminase, ornithine decarboxylase, etc. An appropriate host cell when wild-type DHFR is employed is the CHO cell line deficient in DHFR activity (e.g., ATCC CRL-9096), prepared and propagated. For example, cells transformed with the DHFR selection gene are first identified by culturing all of the transformants in a culture medium that contains methotrexate (Mtx), a competitive antagonist of DHFR. Alternatively, host cells (particularly wild-type hosts that contain endogenous DHFR) transformed or co-transformed with DNA sequences encoding an antibody, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418.

Expression and cloning vectors usually contain a promoter operably linked to the antigen binding protein encoding nucleic acid sequence to direct mRNA synthesis. Promoters recognized by a variety of potential host cells are well known.

Eukaryotic genes generally have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoting sequences for use with yeast hosts include the promoters for 3-phosphoglycerate kinase or other glycolytic enzymes including enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization.

Antigen binding protein transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the antigen binding protein by higher eukaryotes may be increased by inserting an enhancer sequence into the vector. Enhancer sequences include those known from mammalian genes (globin, elastase, albumin, a-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding an antigen binding protein.

In another embodiment there is provided a cell including a vector or nucleic acid described above. The nucleic acid molecule or vector may be present in the genetically modified host cell or host either as an independent molecule outside the genome, preferably as a molecule which is capable of replication, or it may be stably integrated into the genome of the host cell or host.

The host cell of the present invention may be any prokaryotic or eukaryotic cell.

Examples of prokaryotic cells are those generally used for cloning like E. coli or Bacillus subtilis. Furthermore, eukaryotic cells comprise, for example, fungal or animal cells.

Examples for suitable fungal cells are yeast cells, preferably those of the genus Saccharomyces and most preferably those of the species Saccharomyces cerevisiae.

Examples of animal cells are, for instance, insect cells, vertebrate cells, preferably mammalian cells, such as e.g. HEK293, NSO, CHO, MDCK, U2-OS, Hela, NIH3T3, MOLT-4, Jurkat, PC-12, PC-3, IMR, NT2N, Sk-n-sh, CaSki, C33A. These host cells, e.g. CHO-cells, may provide post-translational modifications to the antibody molecules of the invention, including leader peptide removal, folding and assembly of H (heavy) and L (light) chains, glycosylation of the molecule at correct sides and secretion of the functional molecule.

Further suitable cell lines known in the art are obtainable from cell line depositories, like the American Type Culture Collection (ATCC).

In another embodiment there is provided an animal including a cell described above. In certain embodiments, animals and tissues thereof containing a transgene are useful in producing the antigen binding proteins of the invention. The introduction of the nucleic acid molecules as transgenes into non-human hosts and their subsequent expression may be employed for the production of the antigen binding proteins, for example, the expression of such a transgene in the milk of the transgenic animal provide for means of obtaining the antigen binding proteins in quantitative amounts. Useful transgenes in this respect comprise the nucleic acid molecules of the invention, for example, coding sequences for the antigen binding proteins described herein, operatively linked to promoter and/or enhancer structures from a mammary gland specific gene, like casein or beta-lactoglobulin. The animal may be non-human mammals, most preferably mice, rats, sheep, calves, dogs, monkeys or apes.

Compositions

In some examples, an antigen binding protein as described herein can be administered orally, parenterally, by inhalation spray, adsorption, absorption, topically, rectally, nasally, bucally, vaginally, intraventricularly, via an implanted reservoir in dosage formulations containing conventional non-toxic pharmaceutically-acceptable carriers, or by any other convenient dosage form. The term “parenteral” as used herein includes subcutaneous, intravenous, intramuscular, intraperitoneal, intrathecal, intraventricular, intrasternal, and intracranial injection or infusion techniques.

Methods for preparing an antigen binding protein into a suitable form for administration to a subject (e.g. a pharmaceutical composition) are known in the art and include, for example, methods as described in Remington's Pharmaceutical Sciences (18th ed., Mack Publishing Co., Easton, Pa., 1990) and U.S. Pharmacopeia: National Formulary (Mack Publishing Company, Easton, Pa., 1984).

The pharmaceutical compositions of this invention are particularly useful for parenteral administration, such as intravenous administration or administration into a body cavity or lumen of an organ or joint. The compositions for administration will commonly comprise a solution of an antigen binding protein dissolved in a pharmaceutically acceptable carrier, for example an aqueous carrier. A variety of aqueous carriers can be used, e.g., buffered saline and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of an antigen binding protein of the present invention in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Nonaqueous vehicles such as mixed oils and ethyl oleate may also be used. Liposomes may also be used as carriers. The vehicles may contain minor amounts of additives that enhance isotonicity and chemical stability, e.g., buffers and preservatives.

Upon formulation, an antigen binding protein of the present invention will be administered in a manner compatible with the dosage formulation and in such amount as is therapeutically/prophylactically effective. Formulations are easily administered in a variety of dosage forms, such as the type of injectable solutions described above, but other pharmaceutically acceptable forms are also contemplated, e.g., tablets, pills, capsules or other solids for oral administration, suppositories, pessaries, nasal solutions or sprays, aerosols, inhalants, liposomal forms and the like. Pharmaceutical “slow release” capsules or compositions may also be used. Slow release formulations are generally designed to give a constant drug level over an extended period and may be used to deliver an antigen binding protein of the present invention.

WO2002/080967 describes compositions and methods for administering aerosolized compositions comprising antibodies for the treatment of, e.g., asthma, which are also suitable for administration of an antigen binding protein of the present invention.

In another embodiment there is provided a pharmaceutical composition including an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein or conjugate as described above and a pharmaceutically acceptable carrier, diluent or excipient.

Although the invention finds application in humans, the invention is also useful for diagnostic or therapeutic veterinary purposes. The invention is useful for domestic or farm animals such as cattle, sheep, horses and poultry; for companion animals such as cats and dogs; and for zoo animals.

Methods of preparing and administering antigen binding proteins thereof to a subject in need thereof are well known to or are readily determined by those skilled in the art. The route of administration of the antigen binding protein may be oral, parenteral, by inhalation or topical.

While all these forms of administration are clearly contemplated as being within the scope of the invention, a form for administration would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumin), etc.

Preparations for parenteral administration includes sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions, in such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences, Mack Publishing Co., 16th ed. (1980).

Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminium monostearate and gelatin.

Sterile injectable solutions can be prepared by incorporating an active compound (e.g., antigen binding protein) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit. Such articles of manufacture will preferably have labels or package inserts indicating that the associated compositions are useful for treating a subject suffering from, or predisposed disorders.

Dosages and Timing of Administration

Suitable dosages of an antigen binding protein of the present invention will vary depending on the specific an antigen binding protein, the condition to be treated and/or the subject being treated. It is within the ability of a skilled physician to determine a suitable dosage, e.g., by commencing with a sub-optimal dosage and incrementally modifying the dosage to determine an optimal or useful dosage. Alternatively, to determine an appropriate dosage for treatment/prophylaxis, data from the cell culture assays or animal studies are used, wherein a suitable dose is within a range of circulating concentrations that include the ED₅₀ of the active compound with little or no toxicity. The dosage may vary within this range depending upon the dosage form employed and the route of administration utilized. A therapeutically/prophylactically effective dose can be estimated initially from cell culture assays. A dose may be formulated in animal models to achieve a circulating plasma concentration range that includes the IC₅₀ (i.e., the concentration or amount of the compound which achieves a half-maximal inhibition of symptoms) as determined in cell culture. Such information can be used to more accurately determine useful doses in humans. Levels in plasma maybe measured, for example, by high performance liquid chromatography.

In some examples, a method of the present invention comprises administering a prophylactically or therapeutically effective amount of a protein described herein.

The term “therapeutically effective amount” is the quantity which, when administered to a subject in need of treatment, improves the prognosis and/or state of the subject and/or that reduces or inhibits one or more symptoms of a clinical condition described herein to a level that is below that observed and accepted as clinically diagnostic or clinically characteristic of that condition. The amount to be administered to a subject will depend on the particular characteristics of the condition to be treated, the type and stage of condition being treated, the mode of administration, and the characteristics of the subject, such as general health, other diseases, age, sex, genotype, and body weight. A person skilled in the art will be able to determine appropriate dosages depending on these and other factors. Accordingly, this term is not to be construed to limit the present invention to a specific quantity, e.g., weight or amount of protein(s), rather the present invention encompasses any amount of the antigen binding protein(s) sufficient to achieve the stated result in a subject.

As used herein, the term “prophylactically effective amount” shall be taken to mean a sufficient quantity of a protein to prevent or inhibit or delay the onset of one or more detectable symptoms of a clinical condition. The skilled artisan will be aware that such an amount will vary depending on, for example, the specific antigen binding protein(s) administered and/or the particular subject and/or the type or severity or level of condition and/or predisposition (genetic or otherwise) to the condition. Accordingly, this term is not to be construed to limit the present invention to a specific quantity, e.g., weight or amount of antigen binding protein(s), rather the present invention encompasses any amount of the antigen binding protein(s) sufficient to achieve the stated result in a subject.

Effective doses of the compositions of the present invention, for treatment of disorders as described herein vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages may be titrated using routine methods known to those of skill in the art to optimize safety and efficacy.

For treatment of certain disorders with an antigen binding protein, the dosage can range, e.g., from about 0.0001 to 100 mg/kg, and more usually 0.01 to 5 mg/kg (e.g., 0.02 mg/kg, 0.25 mg/kg, 0.5 mg/kg, 0.75 mg/kg, 1 mg/kg, 2 mg/kg, etc.), of the host body weight. For example dosages can be 1 mg/kg body weight or 10 mg/kg body weight or within the range of 1-10 mg/kg, preferably at least 1 mg/kg. Doses intermediate in the above ranges are also intended to be within the scope of the invention. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly. In some methods, two or more antigen binding proteins with different binding specificities are administered simultaneously, in which case the dosage of each antigen binding proteins administered falls within the ranges indicated.

An antigen binding protein disclosed herein can be administered on multiple occasions. Intervals between single dosages can be weekly, monthly or yearly. Intervals can also be irregular as indicated by measuring blood levels of target polypeptide or target molecule in the patient. In some methods, dosage is adjusted to achieve a plasma polypeptide concentration of 1-1000 μg/ml and in some methods 25-300 μg/ml. Alternatively, antigen binding proteins can be administered as a sustained release formulation, in which case less frequent administration is required. Dosage and frequency vary depending on the half-life of the antigen binding protein in the patient. The half-life of an antigen binding protein can also be prolonged via fusion to a stable polypeptide or moiety, e.g., albumin or PEG. In general, humanized antibodies show the longest half-life, followed by chimeric antibodies and nonhuman antibodies. In one embodiment, the antigen binding protein of the invention can be administered in unconjugated form. In another embodiment the antigen binding proteins for use in the methods disclosed herein can be administered multiple times in conjugated form. In still another embodiment, the antigen binding proteins of the invention can be administered in unconjugated form, then in conjugated form, or vice versa.

The dosage and frequency of administration can vary depending on whether the treatment is prophylactic or therapeutic. In prophylactic applications, compositions comprising antibodies or a cocktail thereof are administered to a patient not already in the disease state or in a pre-disease state to enhance the patient's resistance. Such an amount is defined to be a “prophylactic effective dose.” In this use, the precise amounts again depend upon the patient's state of health and general immunity, but generally range from 0.1 to 25 mg per dose, especially 0.5 to 2.5 mg per dose. A relatively low dosage is administered at relatively infrequent intervals over a long period of time. Some patients continue to receive treatment for the rest of their lives.

In therapeutic applications, a relatively high dosage (e.g., from about 1 to 400 mg/kg of binding molecule, e.g., antigen binding protein per dose, with dosages of from 5 to 25 mg being more commonly used for radioimmunoconjugates and higher doses for cytotoxin-drug conjugated molecules) at relatively short intervals is sometimes required until progression of the disease is reduced or terminated, and preferably until the patient shows partial or complete amelioration of symptoms of disease. Thereafter, the patent can be administered a prophylactic regime.

In one embodiment, a subject can be treated with a nucleic acid molecule encoding an antigen binding protein (e.g., in a vector). Doses for nucleic acids encoding polypeptides range from about 10 ng to 1 g, 100 ng to 100 mg, 1 μg to 10 mg, or 30-300 μg DNA per patient. Doses for infectious viral vectors vary from 10-100, or more, virions per dose.

Therapeutic agents can be administered by parenteral, topical, intravenous, oral, subcutaneous, intraarterial, intracranial, intraperitoneal, intranasal or intramuscular means for prophylactic and/or therapeutic treatment, in some methods, agents are injected directly into a particular tissue where CCR6 cells have accumulated, for example intracranial injection. Intramuscular injection or intravenous infusion are preferred for administration of antibody, in some methods, particular therapeutic antibodies are injected directly into the cranium, in some methods, antibodies are administered as a sustained release composition or device.

An antigen binding protein of the invention can optionally be administered in combination with other agents that are effective in treating the disorder or condition in need of treatment (e.g., prophylactic or therapeutic).

In another embodiment there is provided a pharmaceutical composition including an antigen binding protein, immunoglobulin variable domain, antibody, Fab, dab, scFv, diabody, triabody, fusion protein or conjugate as described above, a diluent and optionally a label. For treatment or prevention of psoriasis the pharmaceutical composition is preferably adapted for topical administration.

In certain embodiments, the antigen binding proteins or molecule including same are detectably labelled. Many different labels can be used including enzymes, radioisotopes, colloidal metals, fluorescent compounds, chemiluminescent compounds, and bioluminescent compounds. Fluorochromes (fluorescein, rhodamine, Texas Red, etc.), enzymes (horse radish peroxidase, β-galactosidase, alkaline phosphatase etc.), radioactive isotopes (32P or 125I), biotin, digoxygenin, colloidal metals, chemi- or bioluminescent compounds (dioxetanes, luminol or acridiniums) may be used.

Detection methods depend on the type of label used and include autoradiography, fluorescence microscopy, direct and indirect enzymatic reactions. Examples include Westernblotting, overlay-assays, RIA (Radioimmuno Assay) and IRMA (Immune Radioimmunometric Assay), EIA (Enzyme Immuno Assay), ELISA (Enzyme Linked Immuno Sorbent Assay), FIA (Fluorescent Immuno Assay), and CLIA (Chemioluminescent Immune Assay).

Kits

In another embodiment there is provided a kit or article of manufacture including an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein, conjugate or pharmaceutical composition as described above.

In other embodiments there is provided a kit for use in a therapeutic application mentioned above, the kit including:
a container holding a therapeutic composition in the form of one or more of an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein, conjugate or pharmaceutical composition;
a label or package insert with instructions for use.

In certain embodiments the kit may contain one or more further active principles or ingredients for treatment of a cancer or for preventing a cancer-related complication described above, or a condition or disease associated with CCR6 expression.

The kit or “article of manufacture” may comprise a container and a label or package insert on or associated with the container. Suitable containers include, for example, bottles, vials, syringes, blister pack, etc. The containers may be formed from a variety of materials such as glass or plastic. The container holds a therapeutic composition which is effective for treating the condition and may have a sterile access port (for example the container may be an intravenous solution bag or a vial having a stopper pierceable by a hypodermic injection needle). The label or package insert indicates that the therapeutic composition is used for treating the condition of choice. In one embodiment, the label or package insert includes instructions for use and indicates that the therapeutic composition can be used to treat, prevent or detect a disease or condition characterised by CCR6 expression.

The kit may comprise (a) a therapeutic composition; and (b) a second container with a second active principle or ingredient contained therein. The kit in this embodiment of the invention may further comprise a package insert indicating that the and other active principle can be used to treat a disorder or prevent a complication stemming from cancer. Alternatively, or additionally, the kit may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In certain embodiments the therapeutic composition may be provided in the form of a device, disposable or reusable, including a receptacle for holding the therapeutic composition. In one embodiment, the device is a syringe. The device may hold 1-2 mL of the therapeutic composition. The therapeutic composition may be provided in the device in a state that is ready for use or in a state requiring mixing or addition of further components.

In other embodiments there is provided a kit for use in a diagnostic application mentioned above, the kit including:
a container holding a diagnostic composition in the form of one or more of an antigen binding protein, immunoglobulin variable domain, antibody, Fab, dab, scFv, diabody, triabody, fusion protein or conjugate;
a label or package insert with instructions for use.

The kit may comprise (a) a diagnostic composition; and (b) a second container with a second diagnostic agent or second label contained therein. It may further include other materials desirable from a commercial and user standpoint, including other buffers, diluents, filters etc.

Conditions to be Treated or Diagnosed

In another embodiment there is provided a method for the treatment of a disease or condition characterised by CCR6 expression in an individual including the step of providing an antigen binding protein, immunoglobulin variable domain, antibody, Fab, dab, scFv, diabody, triabody, fusion protein, conjugate or pharmaceutical composition as described above to an individual requiring treatment for said condition. Typically the condition is an inflammatory condition, infection, fibrosis or cancer, especially an epithelial cancer as described herein, or pulmonary disorders such as Chronic obstructive pulmonary disease (COPD), asthma, and Respiratory syncytial virus (RSV).

Other diseases and conditions include various inflammatory conditions. Examples may include a proliferative component. Particular examples include acne, angina, arthritis, aspiration pneumonia, disease, empyema, gastroenteritis, inflammation, intestinal flu, necrotizing enterocolitis, colitis, pelvic inflammatory disease, pharyngitis, pleurisy, raw throat, redness, rubor, sore throat, stomach flu and urinary tract infections, chronic inflammatory demyelinating polyneuropathy, chronic inflammatory demyelinating polyradiculoneuropathy, chronic inflammatory demyelinating polyneuropathy or chronic inflammatory demyelinating polyradiculoneuropathy.

In another embodiment there is provided a use of an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein, conjugate or pharmaceutical composition as described above in the manufacture of a medicament for the treatment of cancer, chronic inflammation, autoimmune disease, infection or fibrosis.

The invention finds application in the diagnosis or treatment of various autoimmune diseases and inflammatory diseases. The inflammatory disorder may be acute or chronic. Inflammatory disorders include cardiovascular inflammation (e.g., atherosclerosis, stroke), gastrointestinal inflammation, hepatic inflammatory disorders, pulmonary inflammation (e.g. asthma, ventilator induced lung injury), kidney inflammation, ocular inflammation (e.g., uveitis), pancreatic inflammation, genitourinary inflammation, neuroinflammatory disorders (e.g., multiple sclerosis, Alzheimer's disease), allergy (e.g., allergic rhinitis/sinusitis, skin allergies and disorders (e.g., urticaria/hives, angioedema, atopic dermatitis, contact dermatitis, psoriasis), food allergies, drug allergies, insect allergies, mastocytosis), skeletal inflammation (e.g., arthritis, osteoarthritis, rheumatoid arthritis, spondyloarthropathies), infection (e.g., bacterial or viral infections oral inflammatory disorders (i.e., periodontis, gingivitis or stomatitis); and transplantation (e.g., allograft or xenograft rejection or maternal-fetal tolerance).

Autoimmune diseases include, for example, Acquired Immunodeficiency Syndrome. (AIDS, which is a viral disease with an autoimmune component), alopecia areata, ankylosing, spondylitis, antiphospholipid syndrome, autoimmune Addison's disease, autoimmune haemolytic, anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune, dysfunction syndrome (CFIDS), chronic inflammatory demyelinating polyneuropathy (CIPD), cicatricial pemphigoid, cold agglutinin disease, crest syndrome, Crohn's disease, Degos' disease, dermatomyositis-juvenile, discoid lupus, essential mixed cryoglobulinemia, fibromyalgia fibromyositis, Graves' disease, Guillain-Barre syndrome, Hashimoto's thyroiditis, idiopathic pulmonary fibrosis, idiopathic thrombocytopenia purpura (ITP), IgA nephropathy, insulin dependent diabetes mellitus, juvenile chronic arthritis (Still's disease), juvenile rheumatoid arthritis, Meniere's disease, mixed connective tissue disease, multiple sclerosis, myasthenia gravis, pemacious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma (progressive systemic sclerosis (PSS), also known as systemic sclerosis (SS)), Sjogren's syndrome, stiff-man syndrome, systemic lupus erythematosus, Takayasu arteritis, temporal arteritis/giant cell arteritis, ulcerative colitis, uveitis, vitiligo and Wegener's granulomatosis.

Preferably, the autoimmune or inflammatory condition is multiple sclerosis, rheumatoid arthritis, skin hypersensitivity such as atopic dermatitis, contact dermatitis, psoriasis, inflammatory bowel disease, uveitis, dry eye disease, Systemic Sclerosis (scleroderma), periodontal disease, vitiligo, SLE/Discoid Lupus/Grave disease, atherosclerosis, asthma, or delayed-type hypersensitivity.

Multiple sclerosis (MS) is an inflammatory disease involving demyelination of myelin sheaths surrounding brain and spinal cord axons. MS symptoms include, but are not limited to scarring of white matter in the brain and/or spinal cord and a wide variety of neurological symptoms, including but not limited to changes in sensation such as loss of sensitivity or tingling, pricking or numbness (hypoesthesia and parasthesia), muscle weakness, clonus, muscle spasms or difficulty in moving; difficulties with coordination and balance (ataxia); problems in speech (dysarthria) or swallowing (dysphagia), visual problems (nystagmus, optic neuritis, etc.), fatigue, acute/chronic pain, and bladder and bowel difficulties. Cognitive impairment of varying degrees and depression are also common. Symptoms of MS usually appear in episodic acute periods of worsening in a gradually progressive deterioration of neurologic function, or in a combination of both.

Rheumatoid arthritis is a chronic systemic inflammatory disorder that may affect many tissues and organs, but principally attacks synovial joints. The process involves an inflammatory response of the synovial capsule around the joints secondary to hyperplasia of synovial cells, excess synovial fluid, and the development of fibrous tissue in the synovia. The pathology of the disease process often leads to the destruction of articular cartilage and ankylosis of the joints. Rheumatoid arthritis can also produce diffuse inflammation in the lungs, pericardium, lung pleura, sclera, and nodular lesions, most common in subcutaneous tissue.

As used herein fibrosis includes any one or more of the following conditions Pulmonary fibrosis, Idiopathic pulmonary fibrosis, Cystic fibrosis, Cirrhosis, Endomyocardial fibrosis, Old myocardial infarction, Atrial Fibrosis, Mediastinal fibrosis, Myelofibrosis, Retroperitoneal fibrosis, Progressive massive fibrosis, Nephrogenic systemic fibrosis, Crohn's Disease, Keloid, Scleroderma/systemic sclerosis, Arthrofibrosis, Peyronie's disease, Dupuytren's contracture, some forms of adhesive capsulitis.

Pre-neoplastic and neoplastic diseases are particular examples to which the methods of the invention may be applied. Broad examples include breast tumors, colorectal tumors, adenocarcinomas, mesothelioma, bladder tumors, prostate tumors, germ cell tumor, hepatoma/cholongio, carcinoma, neuroendocrine tumors, pituitary neoplasm, small 20 round cell tumor, squamous cell cancer, melanoma, atypical fibroxanthoma, seminomas, nonseminomas, stromal leydig cell tumors, Sertoli cell tumors, skin tumors, kidney tumors, testicular tumors, brain tumors, ovarian tumors, stomach tumors, oral tumors, bladder tumors, bone tumors, cervical tumors, esophageal tumors, laryngeal tumors, liver tumors, lung tumors, vaginal tumors and Wilm's tumor.

Examples of particular cancers include but are not limited to adenocarcinoma, adenoma, adenofibroma, adenolymphoma, adontoma, AIDS related. cancers, acoustic neuroma, acute lymphocytic leukemia, acute myeloid leukemia, adenocystic carcinoma, adrenocortical cancer, agnogenic myeloid metaplasia, alopecia, alveolar soft-part sarcoma, ameloblastoma, angiokeratoma, angiolymphoid hyperplasia with eosinophilia, angioma sclerosing, angiomatosis, apudoma, anal cancer, angiosarcoma, aplastic anaemia, astrocytoma, ataxia-telangiectasia, basal cell carcinoma (skin), bladder cancer, bone cancers, bowel cancer, brain stem glioma, brain and CNS tumors, breast cancer, branchioma, CNS tumors, carcinoid tumors, cervical cancer, childhood brain tumors, childhood cancer, childhood leukemia, childhood soft tissue sarcoma, chondrosarcoma, choriocarcinoma, chronic lymphocytic leukemia, chronic myeloid leukemia, colorectal cancers, cutaneous T-cell lymphoma, carcinoma (e.g. Walker, basal cell, basosquamous, Brown-Pearce, ductal, Ehrlich tumor, Krebs 2, Merkel cell, mucinous, non-small cell lung, oat cell, papillary, scirrhous, bronchiolar, bronchogenic, squamous cell, and transitional cell), carcinosarcoma, cervical dysplasia, cystosarcoma phyllodies, cementoma, chordoma, choristoma, chondrosarcoma, chondroblastoma, craniopharyngioma, cholangioma, cholesteatoma, cylindroma, cystadenocarcinoma, cystadenoma, dermatofibrosarcoma-protuberans, desmoplastic-small-round-cell-tumor, ductal carcinoma, dysgerminoam, endocrine cancers, endometrial cancer, ependymoma, esophageal cancer, Ewing's sarcoma, extra-hepatic bile duct cancer, eye cancer, eye: melanoma, retinoblastoma, fallopian tube cancer, fanconi anaemia, fibroma, fibrosarcoma, gall bladder cancer, gastric cancer, gastrointestinal cancers, gastrointestinal-carcinoid-tumor, genitourinary cancers, germ cell tumors, gestationaltrophoblastic-disease, glioma, gynaecological cancers, giant cell tumors, ganglioneuroma, glioma, glomangioma, granulosa cell tumor, gynandroblastoma, haematological malignancies, hairy cell leukemia, head and neck cancer, hepatocellular cancer, hereditary breast cancer, histiocytosis, Hodgkin's disease, human papillomavirus, hydatidiform mole, hypercalcemia, hypopharynx cancer, hamartoma, hemangioendothelioma, hemangioma, hemangiopericytoma, hemangiosarcoma, hemangiosarcoma, histiocytic disorders, histiocytosis malignant, histiocytoma, hepatoma, hidradenoma, hondrosarcoma, immunoproliferative small, opoma, ontraocular melanoma, islet cell cancer, Kaposi's sarcoma, kidney cancer, langerhan's cell-histiocytosis, laryngeal cancer, leiomyosarcoma, leukemia, li-fraumeni syndrome, lip cancer, liposarcoma, liver cancer, lung cancer, lymphedema, lymphoma, Hodgkin's lymphoma, non-Hodgkin's lymphoma, leigomyosarcoma, leukemia (e.g. b-cell, mixed cell, null-cell, t-cell, t-cell chronic, lymphangiosarcoma, lymphocytic acute, lymphocytic chronic, mast-cell and myeloid), leukosarcoma, leydig cell tumor, liposarcoma, leiomyoma, leiomyosarcoma, lymphangioma, lymphangiocytoma, lymphagioma, lymphagiomyoma, lymphangiosarcoma, male breast cancer, malignant-rhabdoid-tumor-of-kidney, medulloblastoma, melanoma, Merkel cell cancer, mesothelioma, metastatic cancer, mouth cancer, multiple endocrine neoplasia, mycosis fungoides, myelodysplastic syndromes, myeloma, myeloproliferative disorders, malignant carcinoid syndrome carcinoid heart disease, medulloblastoma, meningioma, melanoma, mesenchymoma, mesonephroma, mesothelioma, myoblastoma, myoma, myosarcoma, myxoma, myxosarcoma, nasal cancer, nasopharyngeal cancer, nephroblastoma, neuroblastoma, neurofibromatosis, Nijmegen breakage syndrome, non-melanoma skin cancer, non-small-cell-lung-cancer-(nscic), neurilemmoma, neuroblastoma, neuroepithelioma, neurofibromatosis, neurofibroma, neuroma, neoplasms (e.g. bone, breast, digestive system, colorectal, liver), ocular cancers, oesophageal cancer, oral cavity cancer, oropharynx cancer, osteosarcoma, ostomy ovarian cancer, pancreas cancer, paranasal cancer, parathyroid cancer, parotid gland cancer, penile cancer, peripheral-neuroectodermal-tumors, pituitary cancer, polycythemia vera, prostate cancer, osteoma, osteosarcoma, ovarian carcinoma, papilloma, paraganglioma, paraganglioma nonchromaffin, pinealoma, plasmacytoma, protooncogene, rare-cancers-and-associated-disorders, renal cell carcinoma, retinoblastoma, rhabdomyosarcoma, Rothmund-Thomson syndrome, reticuloendotheliosis, rhabdomyoma, salivary gland cancer, sarcoma, schwannoma, Sezary syndrome, skin cancer, small cell lung cancer (scic), small intestine cancer, soft tissue sarcoma, spinal cord tumors, squamous-cell-carcinoma-(skin), stomach cancer, synovial sarcoma, sarcoma (e.g. Ewing's experimental, Kaposi's and mast-cell sarcomas), Sertoli cell tumor, synovioma, testicular cancer, thymus cancer, thyroid cancer, transitional-cell-cancer-(bladder), transitional-cell-cancer-(renal-pelvis-/-ureter), trophoblastic cancer, teratoma, theca cell tumor, thymoma, trophoblastic tumor, urethral cancer, urinary system cancer, uroplakins, uterine sarcoma, uterus cancer, vaginal cancer, vulva cancer, Waldenstrom's-macroglobulinemia and Wilms' tumor.

The invention provides for a method of preventing psoriasis in an individual including the step of providing an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein, conjugate or pharmaceutical composition as described herein to an individual at risk of developing psoriasis. Preferably, the psoriasis is plaque type psoriasis. Prevention of psoriasis may be measured by the absence of erythema, scaling or thickening of the skin.

The invention provides for a method of treating psoriasis or arthritis in an individual including the step of providing an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein, conjugate or pharmaceutical composition as described herein to an individual requiring treatment for psoriasis. Preferably, the psoriasis is plaque type psoriasis.

Treatment of psoriasis may be determined by any clinically or biochemically observable or measurable trait. Preferably, treatment of psoriasis is determined by a reduction in erythema, scaling or thickening of the skin.

Successful treatment of arthritis may be determined by any clinically or biochemically observable or measurable trait. For example, the treatment of rheumatoid arthritis can be assessed by observing an improvement in the subject with respect to the severity of duration of a symptom associated with rheumatoid arthritis. For example, identifying an improvement comprises using a score, a test, or a metric for RA or inflammation, including determining whether a subject has an improved a score for one or more rheumatoid arthritis metrics. The score, the test, or the metric may be selected from the group consisting of one or more of American College of Rheumatology Response Rate (ACR for example ACR20, ACR50, and ACR70), proportion of subjects achieving Low Disease Activity (LDA); Disease Activity Score 28 (DAS28, e.g., based on C-reactive protein); swollen joints; tender joints patient assessments of pain; global disease activity and physical function; physician global assessment of disease activity and acute phase reactant levels; and proportion of subjects achieving ACR70 responder status. Further, the rheumatoid arthritis metric is preferably selected from the group consisting of: Physician Global Assessment of Disease Activity; Patient Reported Outcome; a Health Assessment Questionnaire (HAQ-DI), a patient global assessment of disease activity (VAS)); measurement or presence of an anti-drug antibody (ADA); tender joint count (TJC); swollen joint count (SJC); patient's assessment of pain; Work Instability Scale for Rheumatoid Arthritis; Short Form Health Survey (SF-36); American College of Rheumatology, ACR, (e.g., ACR20, ACR50, and ACR70), proportion of subjects achieving Low Disease Activity (LDA); Disease Activity Score 28 (DAS28, e.g., DAS28 based on C-reactive protein); Clinical Disease Activity Index (CDAI), simple disease activity index (SDAI); and Clinical Remission criteria. The skilled person will be familiar with standard methods for assessing the score for a rheumatoid arthritis metric.

In an embodiment, the method of the present invention reduces the RA metric by at least about 1%, 3%, 5%, 7% 10%, 15%, 20%, 25%, 30%, 35%, 40%, 45%, 50%, 55%, 60%, 65%, 70%, 75%, 80%, 85%, 90%, 95%, 99% or more.

Whether or not the treatment of osteoarthritis has been successful will also be familiar to a person skilled in the art. For example, treatment may be assessed by observing an improvement in one or more metrics selected from the group consisting of Western Ontario and McMaster Universities Arthritis Index (WOMAC), Whole-Organ Magnetic Imaging Score (WORMS), Intermittent and Constant Osteoarthritis Pain (ICOAP) score; 11-point Numeric Rating Score (NRS) score, Physician Global Assessment of Disease Activity, Patient Reported Outcome, a Health Assessment Questionnaire (HAQ-DI), pain levels using a patient global assessment of disease activity (VAS)), measurement or presence of an anti-drug antibody (ADA), tender joint count (TJC), swollen joint count (SJC), patient's assessment of pain, Work Instability Scale for Rheumatoid Arthritis, Short Form Health Survey (SF-36), American College of Rheumatology, ACR, (e.g., ACR20, ACR50, and ACR70), proportion of subjects achieving Low Disease Activity (LDA); Disease Activity Score 28 (DAS28, e.g., DAS28 based on C-reactive protein), Clinical Disease Activity Index (CDAI), simple disease activity index (SDAI), Clinical Remission criteria, and the individual's assessment (for example a questionnaire or a patient's global assessment) is observed in the subject treated according to the present invention.

Further, the treatment of osteoarthritis may be assessed by observing reduced pain associated with osteoarthritis (e.g., moderate-to-severe knee osteoarthritis and/or moderate-to-severe erosive hand osteoarthritis) in an individual. The pain condition may be selected from the group consisting of allodynia, hyperalgesia, and a combination of allodynia and hyperalgesia. Further, the treatment may be assessed by determining knee synovitis/effusion volume, knee bone marrow lesions and extent of osteoarthritis as indicated by magnetic resonance imaging.

Psoriatic arthritis (PsA) refers to chronic inflammatory arthritis which is associated with psoriasis, a common chronic skin condition that causes red patches on the body. About 1 in 20 individuals with psoriasis will develop arthritis along with the skin condition, and in about 75% of cases, psoriasis precedes the arthritis. PsA exhibits itself in a variety of ways, ranging from mild to severe arthritis, wherein the arthritis usually affects the fingers and the spine. PsA is sometimes associated with arthritis mutilans. Arthritis mutilans refers to a disorder which is characterized by excessive bone erosion resulting in a gross, erosive deformity which mutilates the joint.

When the spine is affected, the symptoms of PsA are similar to those of ankylosing spondylitis. Ankylosing spondylitis (AS) is an inflammatory disorder involving inflammation of one or more vertebrae. AS is a chronic inflammatory disease that affects the axial skeleton and/or peripheral joints, including joints between the vertebrae of the spine and sacroiliac joints and the joints between the spine and the pelvis. AS can eventually cause the affected vertebrae to fuse or grow together. Spondyarthropathies, including AS, can be associated with psoriatic arthritis (PsA) and/or inflammatory bowel disease (IBD), including ulcerative colitis and Crohn's disease.

Early manifestations of AS can be determined by radiographic tests, including CT scans and MRI scans. Early manifestations of AS often include sacroiliitis and changes in the sacroiliac joints as evidenced by the blurring of the cortical margins of the subchrondral bone, followed by erosions and sclerosis. Fatigue has also been noted as a common symptom of AS.

Characteristic radiographic features of PsA include joint erosions, joint space narrowing, bony proliferation including periarticular and shaft periostitis, osteolysis including “pencil in cup” deformity and acro-osteolysis, ankylosis, spur formation, and spondylitis (Wassenberg et al. (2001) Z Rheumatol 60:156). Unlike rheumatoid arthritis (RA), joint involvement in PsA is often asymmetrical and may be oligoarticular; osteoporosis is atypical. Although erosive changes in early PsA are marginal as in RA, they become irregular and ill defined with disease progression because of periosteal bone formation adjacent to the erosions. In severe cases, erosive changes may progress to development of pencil in cup deformity or gross osteolysis (Gold et al. (1988) Radiol Clin North Am 26:1195; Resnick et al. (1977)) J Can Assoc Radiol 28:187). Asymmetrical erosions may be visible radiographically in the carpus and in the metacarpophalangeal (MCP), proximal interphalangeal (PIP), and distal interphalangeal (DIP) joints of the hands, but the DIP joints are often the first to be affected. Abnormalities are seen in the phalangeal tufts and at the sites of attachments of tendons and ligaments to the bone. The presence of DIP erosive changes may provide both sensitive and specific radiographic findings to support the diagnosis of PsA. Also, the hands tend to be involved much more frequently than the feet with a ratio of nearly 2:1.

Successful treatment of PsA therefore includes the improvement or resolution of any one or more of the symptoms associated with PsA (including improvement in symptoms of or metrics associated with arthritis, psoriasis, and ankylosing spondylitis).

Dosage amount, dosage frequency, routes of administration etc are described in detail above.

In another embodiment there is provided a method for the diagnosis of cancer or inflammatory disorder including the step of contacting tissues or cells for which the presence or absence of cancer or inflammatory disorder is to be determined with a reagent in the form of an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein, conjugate or diagnostic composition as described above and detecting for the binding of the reagent with the tissues or cells. The method may be operated in vivo or in vitro.

For in situ diagnosis, the antigen binding protein may be administered to the organism to be diagnosed by intravenous, intranasal, intraperitoneal, intracerebral, intraarterial injection or other routes such that a specific binding between an antigen binding protein according to the invention with an epitopic region on the CCR6 may occur. The antibody/antigen complex may conveniently be detected through a label attached to the antigen binding protein or a functional fragment thereof or any other art-known method of detection.

The immunoassays used in diagnostic applications according to the invention and as described herein typically rely on labelled antigens, antibodies, or secondary reagents for detection. These proteins or reagents can be labelled with compounds generally known to those of ordinary skill in the art including enzymes, radioisotopes, and fluorescent, luminescent and chromogenic substances including, but not limited to coloured particles, such as colloidal gold and latex beads. Of these, radioactive labelling can be used for almost all types of assays and with most variations. Enzyme-conjugated labels are particularly useful when radioactivity must be avoided or when quick results are needed. Fluorochromes, although requiring expensive equipment for their use, provide a very sensitive method of detection. Antibodies useful in these assays include monoclonal antibodies, polyclonal antibodies, and affinity purified polyclonal antibodies.

Alternatively, the antigen binding protein may be labelled indirectly by reaction with labelled substances that have an affinity for immunoglobulin, such as protein A or G or second antibodies. The antigen binding protein may be conjugated with a second substance and detected with a labelled third substance having an affinity for the second substance conjugated to the antigen binding protein. For example, the antigen binding protein may be conjugated to biotin and the antigen binding protein-biotin conjugate detected using labelled avidin or streptavidin. Similarly, the antigen binding protein may be conjugated to a hapten and the antigen binding protein-hapten conjugate detected using labelled anti-hapten antibody.

In certain embodiments, immunoassays utilize a double antibody method for detecting the presence of an analyte, wherein, the antigen binding protein is labelled indirectly by reactivity with a second antibody that has been labelled with a detectable label. The second antibody is preferably one that binds to antibodies of the animal from which the antigen binding protein is derived. In other words, if the antigen binding protein is a mouse antibody, then the labelled, second antibody is an anti-mouse antibody. For the antigen binding protein to be used in the assay described herein, this label is preferably an antibody-coated bead, particularly a magnetic bead. For the antigen binding protein to be employed in the immunoassay described herein, the label is preferably a detectable molecule such as a radioactive, fluorescent or an electrochemiluminescent substance.

An alternative double antibody system, often referred to as fast format systems because they are adapted to rapid determinations of the presence of an analyte, may also be employed within the scope of the present invention. The system requires high affinity between the antigen binding protein and the analyte. According to one embodiment of the present invention, the presence of the CCR6 is determined using a pair of antigen binding proteins, each specific for CCR6 protein. One of said pairs of antigen binding proteins is referred to herein as a “detector antigen binding protein” and the other of said pair of antigen binding proteins is referred to herein as a “capture antigen binding protein”. The antigen binding protein of the present invention can be used as either a capture antigen binding protein or a detector antigen binding protein. The antigen binding protein of the present invention can also be used as both capture and detector antigen binding protein, together in a single assay. One embodiment of the present invention thus uses the double antigen binding protein sandwich method for detecting CCR6 in a sample of biological fluid. In this method, the analyte (CCR6 protein) is sandwiched between the detector antigen binding protein and the capture antigen binding protein, the capture antigen binding protein being irreversibly immobilized onto a solid support. The detector antigen binding protein would contain a detectable label, in order to identify the presence of the antigen binding protein-analyte sandwich and thus the presence of the analyte.

Exemplary solid phase substances include, but are not limited to, microtiter plates, test tubes of polystyrene, magnetic, plastic or glass beads and slides which are well known in the field of radioimmunoassay and enzyme immunoassay. Methods for coupling antigen binding proteins to solid phases are also well known to those of ordinary skill in the art. More recently, a number of porous material such as nylon, nitrocellulose, cellulose acetate, glass fibers and other porous polymers have been employed as solid supports.

It will be understood that the invention disclosed and defined in this specification extends to all alternative combinations of two or more of the individual features mentioned or evident from the text or drawings. All of these different combinations constitute various alternative aspects of the invention.

The examples that follow are intended to illustrate but in no way limit the present invention.

### EXAMPLES

### Example 1—Generation of Anti-Human CCR6 Monoclonal Antibody

Monoclonal antibodies reactive with human CCR6 (hCCR6) were generated by immunising C57BL/6 mice with 2×10⁷ L1.2/hCCR6 transfected cells stimulated 20 hours prior to harvest with 5 mM butyric acid and emulsified in Complete Freund's Adjuvant (1st immunization intraperitoneal) or Incomplete Freund's Adjuvant (2nd-6th immunizations intraperitoneal), for a total five to six times at 2-wk intervals. The final immunisation was injected intravenously in PBS. Four days later, the spleen was removed and cells were fused with the SP2/0 cell line using standard methods. Hybridomas were grown in DMEM (Gibco/Invitrogen) containing 10% Fetalclone (HyClone), 1×HAT supplement (Sigma Aldrich) plus mouse IL-6. After 10-14 days growth culture supernatant was taken for initial screening.

Monoclonal antibodies reactive with CCR6 were identified using human CCR6 transfected L1.2 cells, and untransfected L1.2 cells, or L1.2 cells transfected with unrelated or closely receptors such as hCXCRI, hCXCR2 or hCXCR3 using immunofluorescent staining and analysis using a FACSCalibur (BD Biosciences). Monoclonal antibody staining of cells was performed using standard procedures as described previously (Lee et al., 2006, Nat. Biotech. 24:1279-1284).

Production of antibodies involved growing hybridomas in tissue culture flasks and harvesting the culture medium. For some experiments, the concentration of antibody in the culture supernatant was sufficient to proceed without further purification. Production of selected antibodies was scaled up and monoclonal antibodies were purified by protein G chromatography, concentrated and buffer exchanged into PBS. Monoclonal antibody concentration was determined using a total IgG ELISA.

L1.2 transfectants expressing high levels of hCCR6 were used to immunize mice, and approximately 40 monoclonal antibodies were initially identified via flow cytometry that reacted with L1.2 cells transfected with hCCR6, of which approximately 10 reacted specifically with L1.2/hCCR6 transfectants but not with untransfected L1.2 cells or with L1.2 cells transfected with the closely related receptors hCXCRI, hCXCR2 or hCXCR3 (FIG. 3).

To ensure clonality, selected hybridomas were subcloned using dilution plating into a 384-well plate (shown in Table 7 below). The specificity of cross-reactivity of the subclones was confirmed by flow cytometry with L1.2/hCCR6 transfectants and untransfected L1.2 cells.

| | # hCCR6 +ve clones | Selected | |
|---|---|---|---|
| Hybridoma | (transfectants) | clone # | Isotype |
| AB1 | 6/28 | AB1-O17 | IgG1 |
| AB3 | 6/11 | AB3-N21 | IgG1 |
| AB8 | 4/30 | ** | |
| AB9 | 9/9 | AB9-K16 | IgG1 |
| AB2 | 6/16 | AB2-O17 | IgG1 |
| AB5 | 4/20 | AB5-M19 | IgG1 |
| AB10 | 5/16 | AB10-N16 | IgG1 |
| AB11 | 21/21 | AB11-M9 | IgG1 |
| AB6 | 14/23 | AB6-K5 | IgG2b |
| AB4 | 2/21 | AB4-K15 | IgG2b |
| AB12 | 2/15 | ** | |

** all subclones negative on human lymphocytes

### Example 2—Sequencing of Anti-Human CCR6 Variable Region Genes

Total RNA from the anti-hCCR6 hybridomas were used to synthesize cDNA for sequencing analysis. The variable region genes were amplified by RT-PCR using primers annealing to the mouse light (mIgCk) and heavy (mIgG2a) constant regions, and the variable heavy chain (VH) and variable light chain (VL) genes were sequenced.

### Example 3—Competitive Inhibition of Ligand Binding to CCR6 by Monoclonal Antibodies

For ligand binding analysis, recombinant human CCL20 (MIP3α) (“ligand”) was obtained from Peprotech (New Jersey, USA). 125I-Bolton-Hunter-labelled MIP3α was purchased from Perkin-Elmer (Boston, MA, USA), with a specific activity of 2200 Ci/mM. Cells were washed once in binding buffer (50 mM Hepes, pH 7.5, 1 mM CaCl, 5 mM MgCb, 0.5% BSA) and resuspended in binding buffer at a concentration of 2.5×106 cells/ml. Cold Purified monoclonal antibody or diluted hybridoma culture medium (cold competitor) was added to a 96-well plate followed by an equal volume (40 μl) binding buffer containing 1×105 cells. Cells and competitor were preincubated at room temperature for 15 min. Then radiolabeled ligand (final concentration 0.5-2 nM) was added to each well to give a final reaction volume of 120 μl. After a 60-min incubation at room temperature, the cells were washed three times with 1 ml of binding buffer containing 150 mM NaCl. The radioactivity (amount of bound label) in the cell pellets was counted in a TopCount liquid scintillation counter (Packard). Non-specific background binding was calculated by incubating cells without radiolabelled-ligand. Samples were assayed in duplicate.

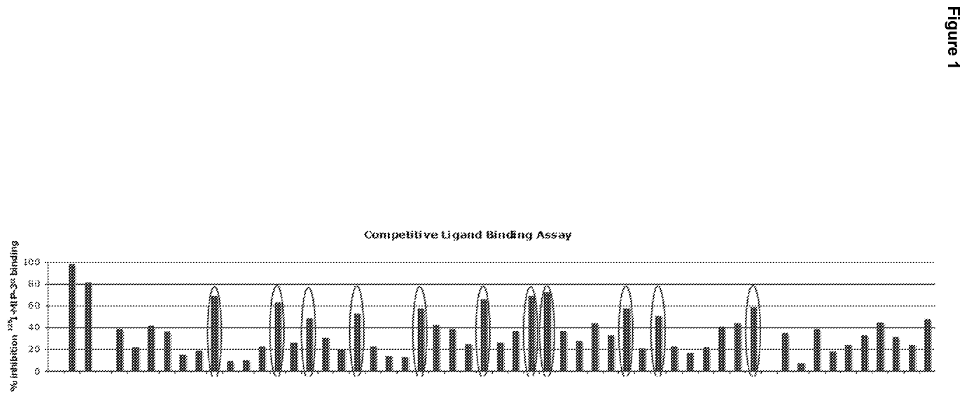

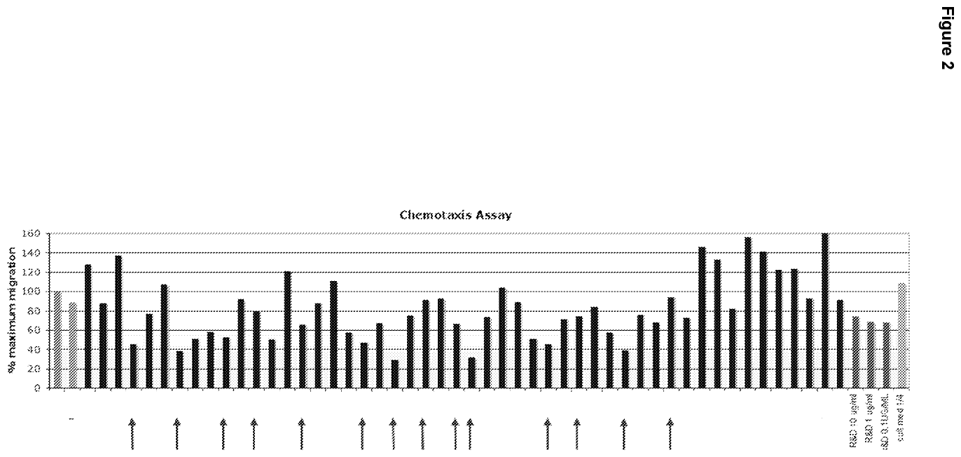

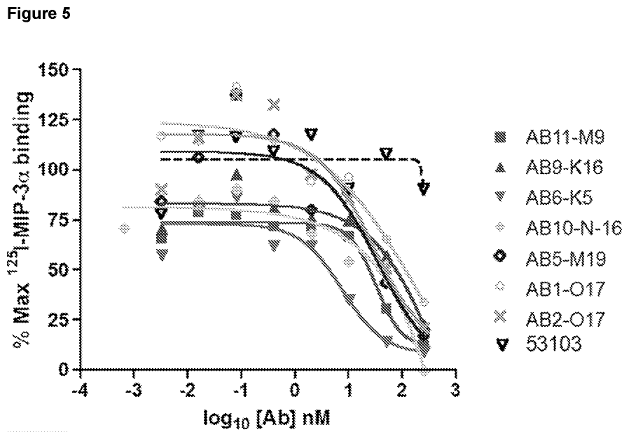

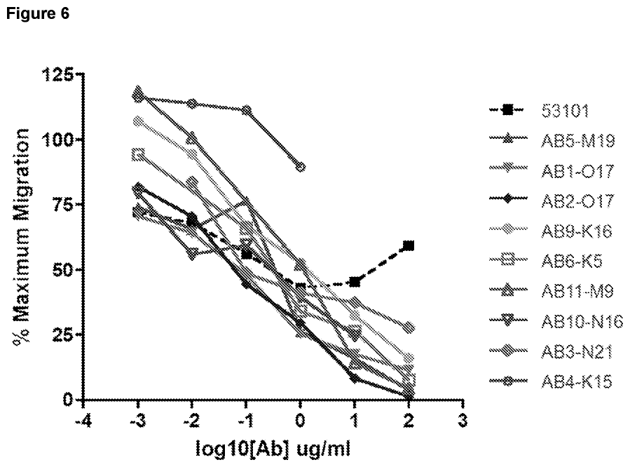

Initially, a panel of anti-CCR6 monoclonal antibodies identified as binding to human CCCR6 transfectants was screened for their ability to competitively inhibit binding of 125I-labelled ligand to hCCR6/L1.2 transfectants treated with 5 mM butyric acid for 20 hr prior to assay. After incubation and washing the amount of label bound to cells was measured and the percentage inhibition determined by comparison to a control reaction with no added antibody (FIGS. 1 and 5).

### Example 4—Transfectant Chemotaxis Assay

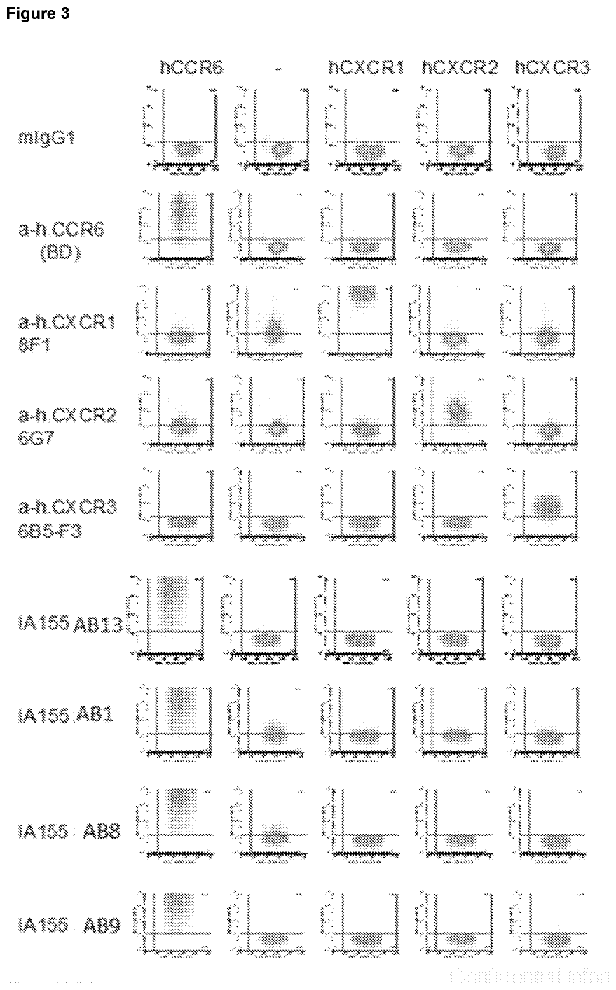

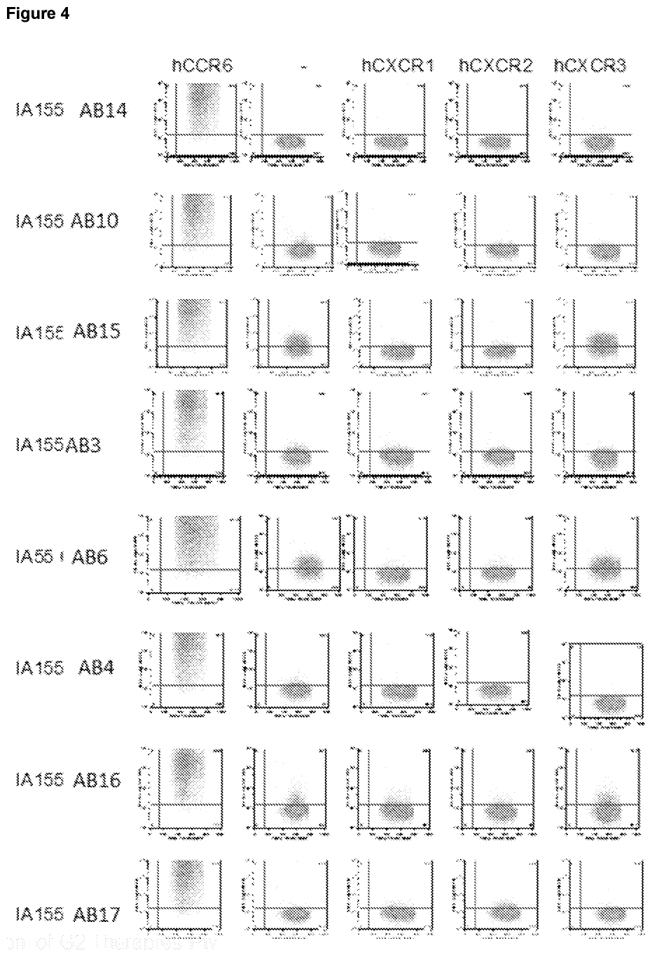

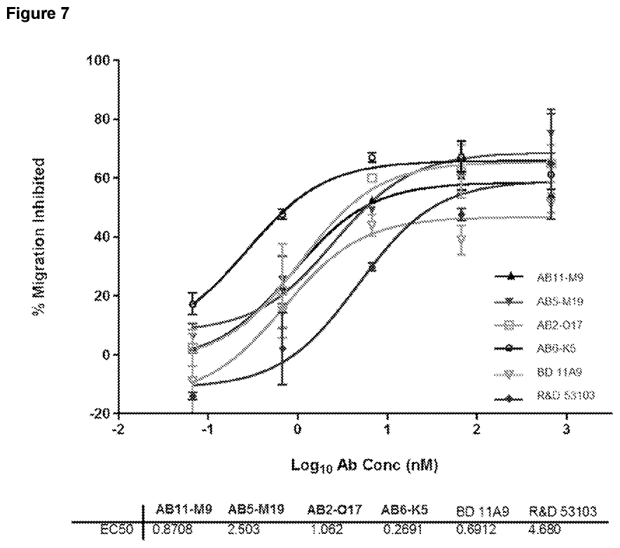

Human CCR6 transfected L1.2 cells were spun down and washed in migration medium (MM=RPMI 1640, 0.5% BSA) and resuspended at 107 cells/ml. Tissue culture inserts (Becton Dickinson & Co., Mountain View, Calif.) were placed in each of the wells of 24-well tissue-culture plates, forming an upper and lower chamber separated by a polyethylene terephtalate membrane bearing 3-mm-diameter pores. Chemotactic MIP3α (diluted in assay medium) was added to 600 μl of assay medium in the 24-well tissue culture plates. One million cells in 100 μl were pre-incubated for 30 mins with the antibodies. The purified mAb was added to the upper chamber in the wells and the cells were allowed to migrate through to the lower chamber in an 5% CO2, 37° C. incubator for 18 h. The inserts were removed after migration and the cells were counted by the LSRII cytometer (BD Biosciences). Relative cell counts were obtained by acquiring events for a set time period of 30 seconds. This method was found to be highly reproducible, and enabled gating on the live cells and the exclusion of debris (FIGS. 2, 6 and 7).

### Example 5—Epitope Mapping

Epitope mapping studies were performed to determine the region within CCR6 that is recognized by anti-CCR6 mAb. Initially, biotinylated peptides corresponding to the N-terminal region and the first, second and third extracellular loops of human CCR6 were used in an ELISA. The results of this preliminary mapping study indicated that all the anti-human CCR6 mAb recognized the N-terminal region of CCR6.

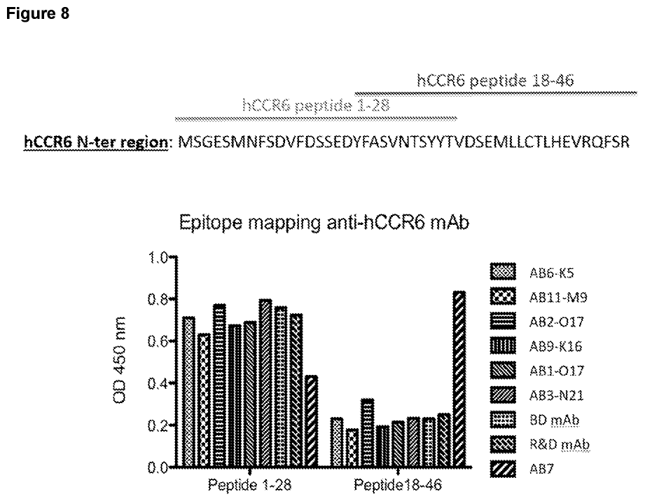

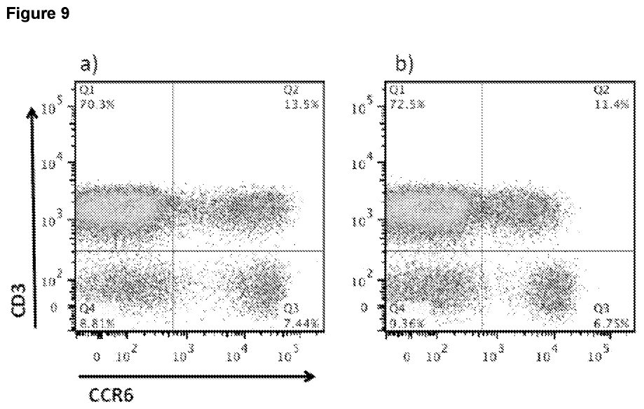

Two overlapping biotinylated peptides spanning the entire N-terminal region of human CCR6 were then synthesized and used in more defined anti-CCR6 mAb epitope mapping studies. Peptide 1 (MSGESMNFSDVFDSSEDYFASVNTSYYT, SEQ ID NO: 2) corresponds to amino acid position 1-28 of the human CCR6 and Peptide 2 (YFASVNTSYYTVDSEMLLCTLHEVRQFSR, SEQ ID NO: 101) corresponds to amino acid position 18-46 of the human CCR6. Briefly, multiwell plates were coated with streptavidin and washed before the biotinylated peptides were added to separate wells and incubated to facilitate binding of the peptides to the plate. Different anti-human CCR6 antibodies were then tested by adding the respective antibodies to the wells of the plate and incubating the plate. An isotype control and buffer only were included as negative controls. Following washing, appropriate conjugated antibodies were added and the plates were incubated. The plates were washed again and binding of the antibodies to the immobilised peptides was visualised (FIGS. 8 and 9).

Results: Most of the anti-hCCR6 antibodies recognize the N-terminal region of human CCR6. More precisely, the majority of antibodies react with a region involving the first 28 AA. Only the clone AB7 recognize an epitope involving the AA 18 to 46.

### Example 6—Generation of Humanized AB6 mAb

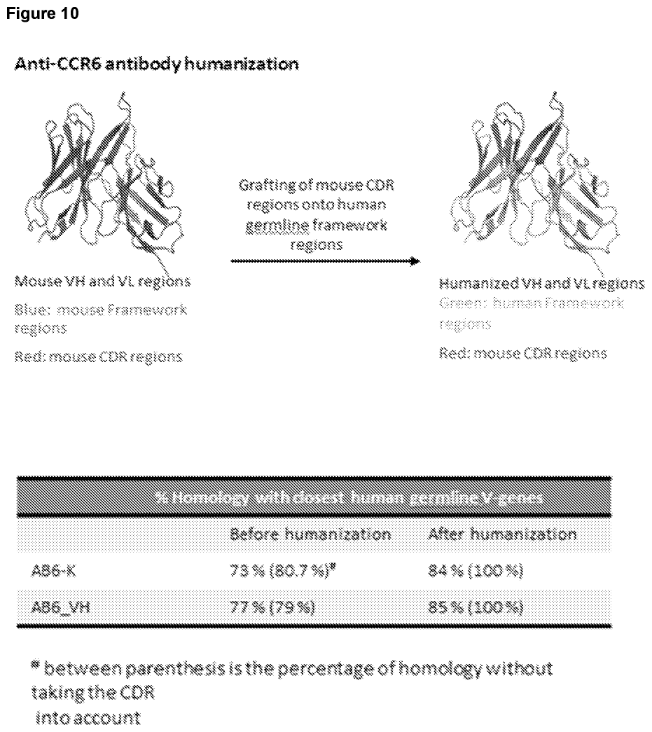

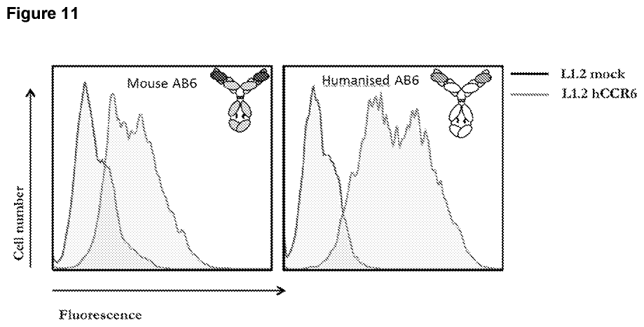

Humanized AB6 mAbs were generated by transferring the CDRs of the AB6 mAb (CDR-H1; CDR-H2; CDR-H3; CDR-L1; CDR-L2; and CDR-L3) onto human framework regions using standard molecular techniques (FIGS. 10 and 11). IMGT/V-QUEST and IMGT/Junctions analysis tools were used to identify human germline genes in which sequences from the variable regions of both the heavy and light chains were closely aligned with those of murine antibody. Framework sequences of these selected human germline genes were used as acceptor sequences for the mouse AB6 CDRs (IGHV3-48*02 and IGKV2-28*01 human genes according to IMGT database). However, murine residues were retained in the critical “Vernier” zone. The humanized VH and VL genes, which were also codon optimized for expressed in CHO cells, were synthesized by Genescript.

Fc variants of the humanized AB6 antibody were generated by standard site directed mutagenesis techniques in order to enhance or decrease antibody-dependent cell-mediated cytotoxicity (ADCC). The triple mutation S239D/A330L/I332E (Eu numbering system), known as “3M”, was introduced into the Fc region to enhance ADCC, resulting in the humanized AB6-3MFc antibody. The triple mutation L234F/L235E/P331S (Eu numbering system) was also introduced into the Fc region to reduce ADCC, resulting in the humanized AB6-Fc-KO antibody (3SFC).

### Example 7—Antibody Dependent Cell Cytotoxicity ADCC Analysis by Flow Cytometry

The capacity of hAB6 (3MFc and 3SFc) to induce effector cell—dependent lysis of L1.2 hCCR6 transfected cells was evaluated by flow cytometry. Briefly, L1.2 hCCR6 transfected cells were labeled with membrane dye, PKH26, to allow discrimination when incubated with effector cells and antibodies. Labeled target cells were washed 3 times with culture medium and resuspended in culture medium at a concentration of 1×106/ml. Labeled target cells were dispensed in round-bottomed 96-well plates (1×105 in 100 μl/well) and preincubated with 20 μg/ml of hAB6 or human IgG1 isotype control (Sigma) at 37° C. for 30 minutes. PBMCs were prepared from heparinized blood (obtained from healthy individuals) by centrifugation on Ficoll. Thereafter, PBMCs (effector cells) were added to the 96-well plates containing target cells with effector cell:target cell (E:T) ratios of 1:50 and were incubated at 37° C. for 3 hours. Just before analysis on a LSRII cytometer (BD Biosciences), TO-PRO 3 iodide was added to detect cell death.

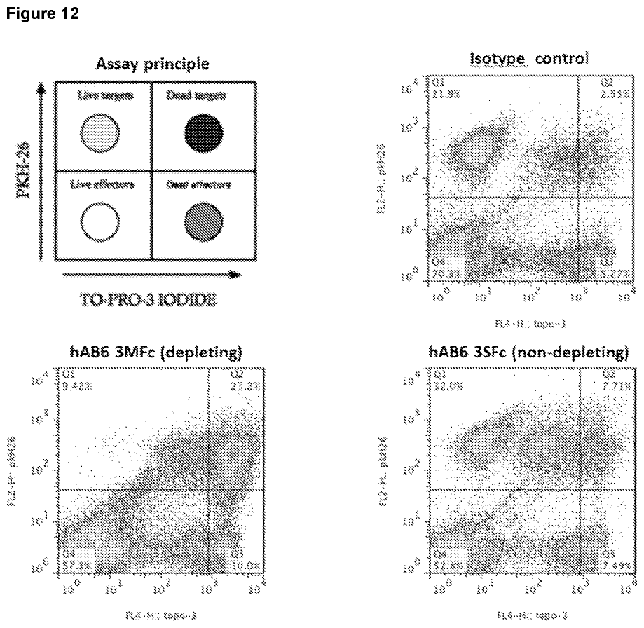

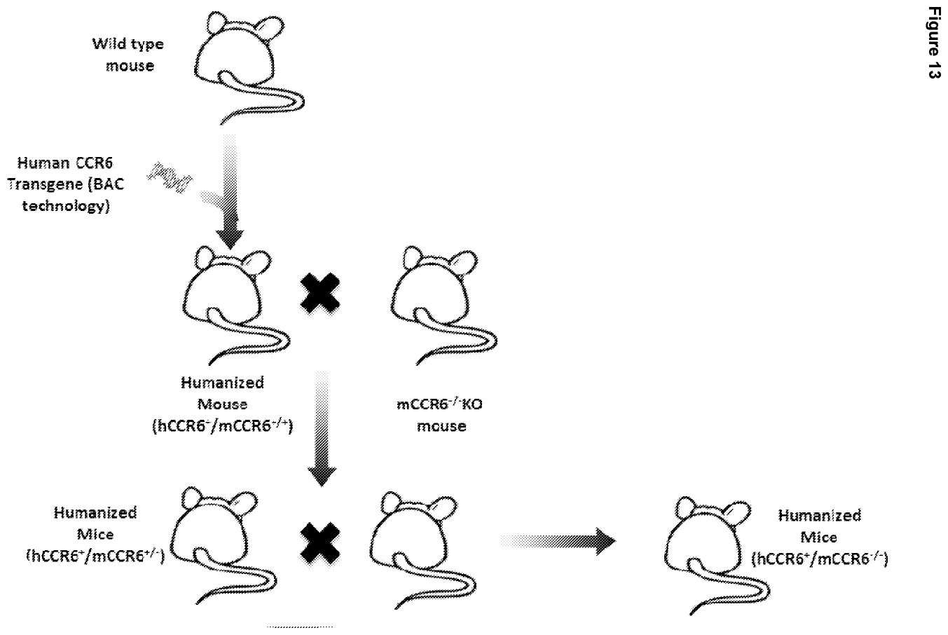

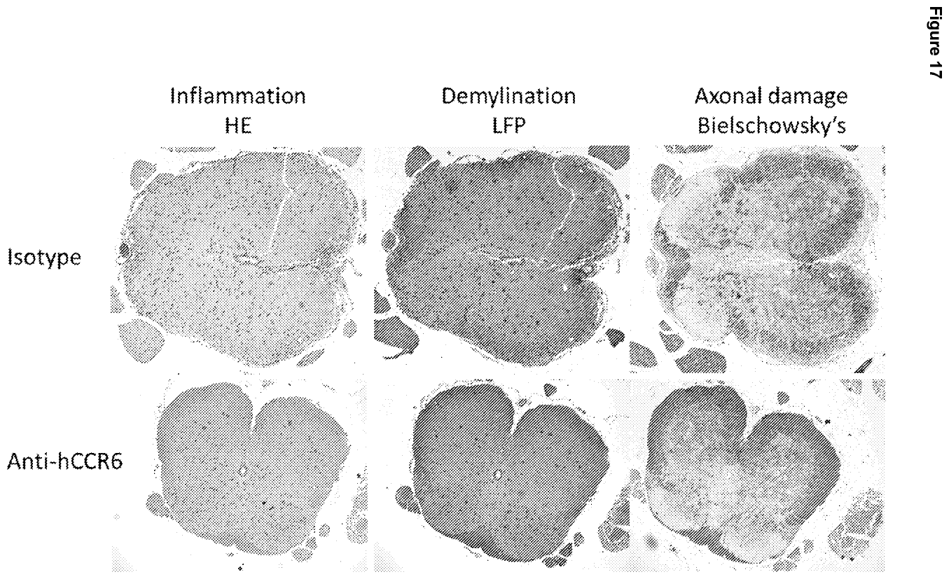

Results: Humanized anti-hCCR6 antibody effector functions can be engineered for depletion (3MFc) or blocking (3SFc) of human CCR6 positive cells (FIGS. 12 and 16).

### Example 8—Generation of Humanized hCCR6 Transgenic Mice

Human CCR6 transgenic mice were created using the BAC clone RP11-319P19 containing the human CCR6 gene. The BAC was linearized by restriction endonuclease. The human CCR6 gene fragment was purified and injected into one-day-old C57BL/6 embryos via pronuclear microinjection. The embryos were then implanted into ICR surrogate females and the resulting progeny were screened by PCR for the presence of the human CCR6 transgene. hCCR6+ mice were crossed to mCCR6−/− mice to generate hCCR6+/mCCR6−/− lines (FIG. 13).

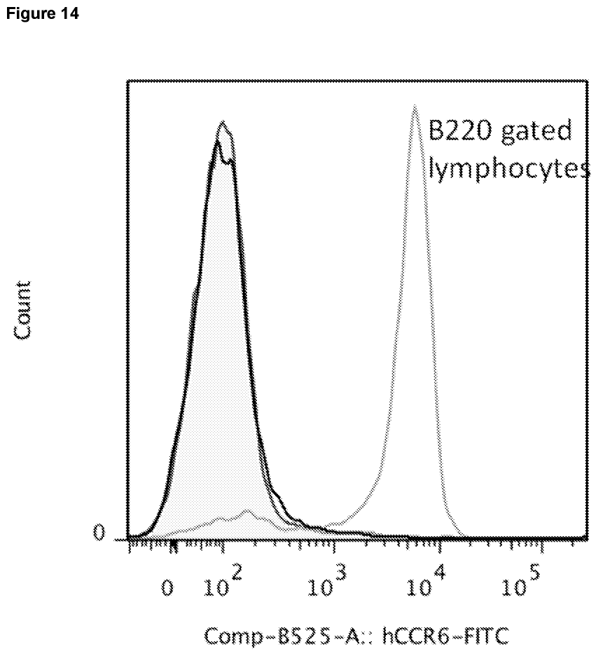

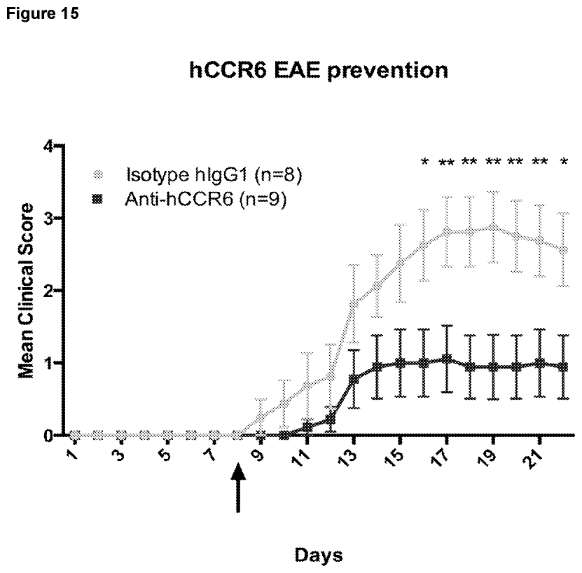

Results: To study human CCR6 in the context of anti-human CCR6 antibody anti-inflammatory activity, we expressed hCCR6—driven by its endogenous promoter to reproduce the characteristically in vivo expression pattern of hCCR6—in a mouse. Human bacterial artificial chromosome (BAC) clones encoding the hCCR6 gene and its regulatory regions were introduced as a transgene into mice. Transgenic mice showed surface expression of this human chemokine receptor on lymphocytes in the peripheral blood, and spleen, resembling the human expression pattern of CCR6 (FIG. 14).

### Example 9—In Vivo Effect of Humanized AB6 mAb on Experimental Autoimmune Encephalomyelitis (EAE)

Several studies have demonstrated that CCR6, a receptor preferentially expressed by CD4+ Th17 cells, as well as its corresponding ligand (CCL20, MIP-3α), are involved in multiple sclerosis. Accordingly, experiments were performed to determine whether blocking CCR6+ cells using the humanized AB6 mAb, would in a EAE mouse model would result in immunosuppression and amelioration of the disease outcomes.

To induce EAE, 8 to 12-week-old female hCCR6 Tg C57BL/6 mice were injected subcutaneously with 100 μg recombinant mouse MOG 1-117 (Clements C S et al. Proc Natl Acad Sci USA 2003; 100: 11059-11064) in complete Freund adjuvant (DIFCO Laboratories, Detroit, MI). After immunization and 48 hours later, mice received an intravenous injection of 200 ng pertussis toxin. Individual animals were observed daily and clinical scores were assessed as follows: 0=no clinical disease, 1=loss of tail tone only, 2=mild monoparesis or paraparesis, 3=severe paraparesis, 4=paraplegia and/or quadraparesis, and 5=moribund or death. The timing of the administration of the antibodies are prior to the clinically observable onset of disease and therefore provides the opportunity to test whether the anti-CCR6 antibodies can delay onset or reduce the severity of the disease.

One single injection of PBS, the purified humanized anti-CCR6 mab or an isotype control (5 mg/kg) was performed on day 8 post-immunization.

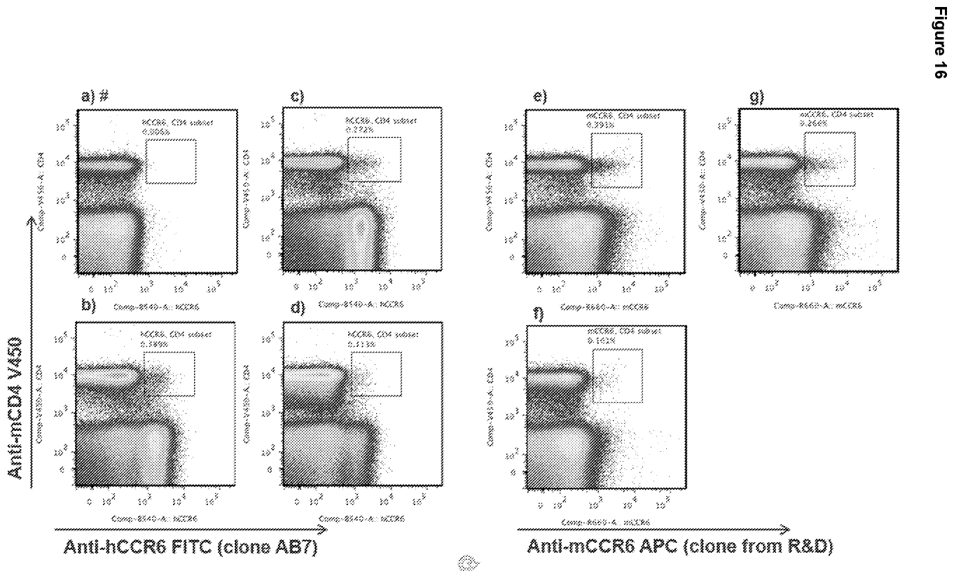

Results: Treatment of hCCR6 transgenic mice with a single injection of the humanized anti-hCCR6 antibody (hAB6-3SFc) significantly reduced the development of EAE (FIG. 15).

### Example 10—Histological Analysis of Animals from EAE Study Described in Example 9 Above

Representative stained histological sections of spinal cords from immunized animal treated with isotype or anti-ccr6 mAb (preventive study). Serial sections were stained with hematoxylin and eosin (H&E) to determine the degree of inflammatory cell infiltrates, luxol fast blue (LFB: arrow heads) to establish myelin integrity and Bielschowski silver stain to ascertain for axonal loss and damage (arrows) (see FIG. 17). Administration of the hAB6 antibody resulted in blocking the infiltration of inflammatory cells, such as T cells, B cells and macrophages. The antibody also reduced myelin degradation and prevented axonal loss and damage.

### Example 11—In Vivo Effect of Humanized AB6 mAb on Experimental Autoimmune Encephalomyelitis (EAE)

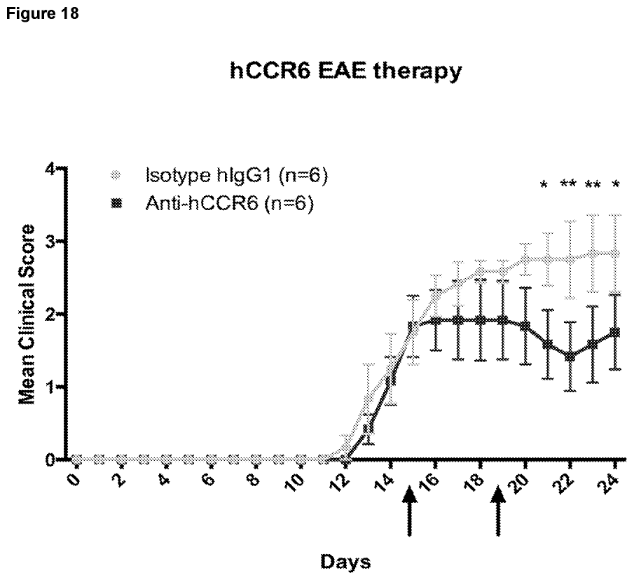

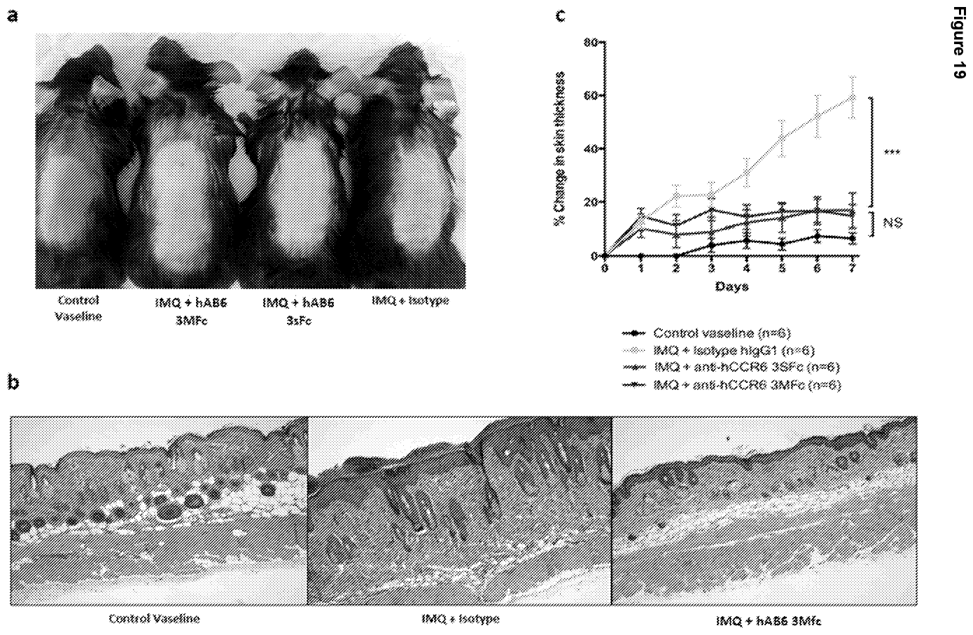

Eight to 12-week-old female hCCR6 Tg mice were injected subcutaneously with 100 μg rMOG 1-117 in complete Freund adjuvant. After immunization and 48 hours later, mice received an intravenous injection of 200 ng pertussis toxin. When an average clinical score of 2 was reached (Day 15), the animals were treated with 2 mg/kg of humanized anti-hCCR6 or humanized anti-hCXCR3 mab (isotype group). Animals received a second injection on day 19. Results are shown in FIG. 18.

Results: Administration of humanized AB6 mAb at day 15 lead to stabilization of clinical disease and subsequent administration at day 19 resulted in an observable improvement in monoparesis or paraparesis, suggesting that targeting CCR6 can not only stabilize disease but can reverse symptoms of diseases characterised by demyelination and/or mononuclear cell infiltration into the CNS, such as multiple sclerosis.

### Example 12—In Vivo Effect of Humanized AB6 mAb on IMIQUIMOD (IMQ)-Induced Psoriasis Model

IMQ-induced skin inflammation in mice phenotypically resembles psoriasis (IMIQUIMOD-induced psoriasis model (van der Fits, et al. The Journal of Immunology 2009 vol. 182 no. 9 5836-5845)). Upon application of IMQ to the skin, the site of application, typically the back, will display signs of erythema, scaling, and thickening. IMQ-treated skin also shows increased epidermal thickening which is caused by hyperproliferation of keratinocytes (van der Fits, et al. 2009). IMQ treatment in a mouse model results in hyperproliferative keratinocytes and a disturbed epidermal differentiation (parakeratosis) which is commonly displayed by the retention of nuclei in the stratum corneum, the absence of a granular layer, and an altered involucrin expression pattern (van der Fits et al. 2009). These observable and measurable traits match the characteristic histological picture of plaque type psoriasis.

Preventative Study

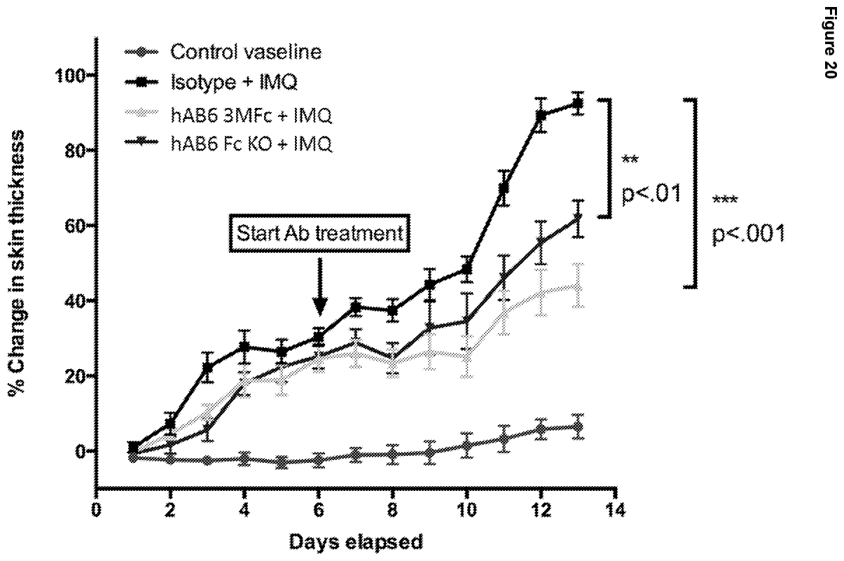

hCCR6 Tg mice were treated daily with IMQ cream or control cream (vaseline) on the shaved back skin. FIG. 19 shows a phenotypical presentation of mouse back skin after 7 days of treatment with treatment beginning on the same day as the first application of IMQ cream (i.e. day 0). Mice were treated daily with Isotype control antibody (5 mg/kg) displayed epidermal thickening, erythema and scaling (far right), however treatment with humanized anti-hCCR6 mab, hAB6, (3Mfc or 3SFc at 5 mg/kg) prevented formation of epidermal thickening, erythema and scaling. (B) IMQ treatment alters keratinocyte proliferation and differentiation. Mice were treated for 7 days with IMQ or vaseline cream. H&E staining of back skin of mice (Vaseline control; IMQ+Isotype or IMQ+hAB6 3MFc) showed that IMQ causes hyperproliferation of keratinocytes, and altered differentiation, which was prevented by hAB6 3MFc. (C) IMQ induced back skin thickening. Anti-hCCR6, either with 3Mfc or 3SFc, significantly reduced back skin thickening compared to isotype control. Results suggest that the anti-CCR6 antibodies prevent formation of psoriasis and that this effect is independent of Fc function.

Therapeutic Study

hCCR6 Tg mice were treated daily with IMQ cream or control cream (vaseline) on the shaved back skin and treated daily with Isotype control antibody (5 mg/kg) or humanized anti-hCCR6 mab (3Mfc or 3SFc at 5 mg/kg) beginning on day 6 after the first application of IMQ cream. This therapeutic study measured IMQ induced back skin thickening with both anti-hCCR6 antibodies, hAB6 3Mfc or Fc KO (3SFc) significantly reduced back skin thickening compared to isotype control. These results show that the anti-CCR6 antibodies of the invention can treat psoriasis and slow its progression (FIG. 20).

### Example 13—In Vitro ADCC Assay

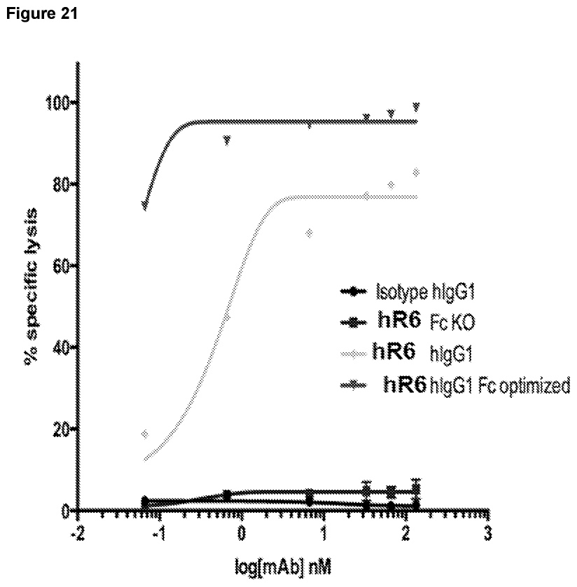

The cytolytic capacity of hAB6 depleting antibodies (IgG1 and IgG1 Fc optimised), was compared to non-depleting hAB6 (Fc KO) and isotype control. hAb6 depleting antibodies were shown to have significantly increased cell killing capacity compared to non-depleting or control (FIG. 21).

### Example 14—Therapeutic Arthritis Study

Human CCR6 Transgenic mice were injected (i.p.) with 200 μL of K/BXN serum on day 0 and 1. The development of arthritis was assessed by measuring the ankle thickness and clinical index score every day until the experimental endpoint. When mice exhibiting symptoms of arthritis and cumulative clinical score reached 4 (on day 4), mice split were into two groups: those injected with isotype control mAb antibody; those that were injected with anti-hCCR6-FcKO antibody (blue); and those injected with anti-hCCR6-depleting antibody (green) at 20 mg/kg of body weight, followed by 5 mg/kg every other day for 1 week. As a control, mice that don't express human CCR6 (WT) were injected intraperitoneally with 200 μL of K/BXN serum on day 0 and 1 and treated with anti-hCCR6-depleting antibody (red).

Representative images of mouse ankles at the experimental endpoint.

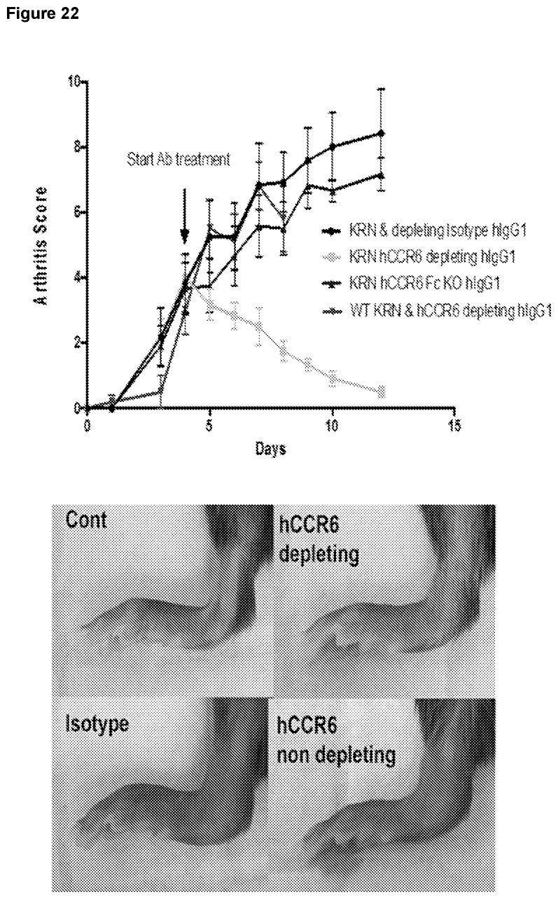

Results, shown in FIG. 22, demonstrate that anti-CCR6-depleting antibody significantly reduces symptoms and signs of arthritis in mice.

### Example 15—Affinity Maturation of h6H12

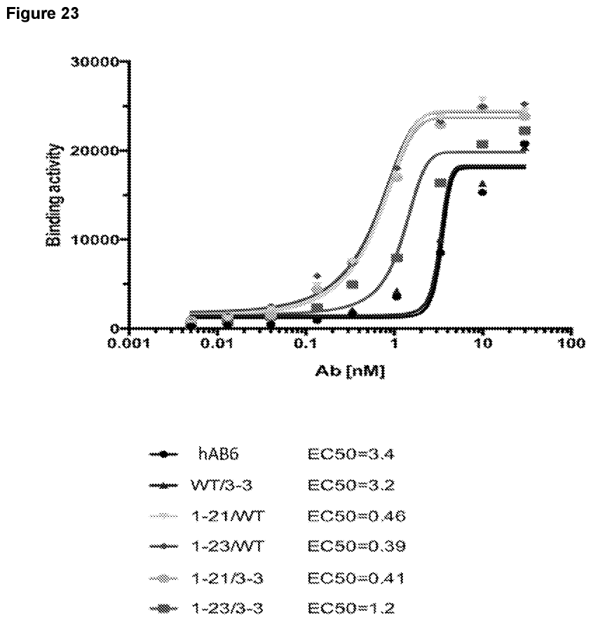

The VH and VK nucleic acid sequences of hAB6 underwent mutation to produce VK sequences 1-21, 1-23 and VH sequence 3-3. Those sequences were arranged to produce various mutated antibodies (FIG. 23) with either wild-type hAB6 VH and VL, and/or various combinations of mutated 1-21, 1-23 VH or 3-3 VL, respectively. The antibodies as described herein are shown as VH/VL: WT/3-3, 1-21/WT, 1-23/WT, 1-21/3-3, and 1-23/3-3. The affinity of these resulting antibodies were tested by flow cytometry cell binding assay on human CCR6 L1.2 cells.

Binding characteristics of hAB6 and mutants hAB6-IgG1 to human CCR6 was performed using a FACS binding assay using cell line expressing human CCR6 (L1.2 human CCR6). Approximately 2.5 10⁵ hCCR6 L1.2 cells/test were washed with FACS binding buffer (FBB) (PBS, 0.5% BSA, 0.1% NaN3 (pH 7.4)), and stained with hAB6 or hAB6 mutants (4.4 μg/ml) and serial 3-fold dilutions of antibodies. After 1 h on ice, the cells were washed with FBB (pH 7.4) and with an anti-human Fc antibody PE-conjugated (Jackson ImmunoResearch). After 30 min on ice, the cells were washed 3 times with FBB (pH 7.4), and resuspended in 1% formaldehyde, and analyzed using a FACS LSR flow cytometer (BD Immunocytometry Systems). EC₅₀ values were calculated using GraphPad Prism. EC50 values (in nM) were 3.4 (hAB6), 3.2 (WT/3.3), 0.46 (1-21/WT), 0.39 (1-23/WT), 0.41 (1-21/3-3) and 1.2 (1-23/3.3).

### Example 16—Therapeutic Scleroderma Study

The therapeutic efficacy of anti-CCR6 depleting antibodies was assessed in a model of bleomycin-induced scleroderma. Briefly, C57/BL6 mice at age 6 weeks were acclimatized for 7 days at the animal house.

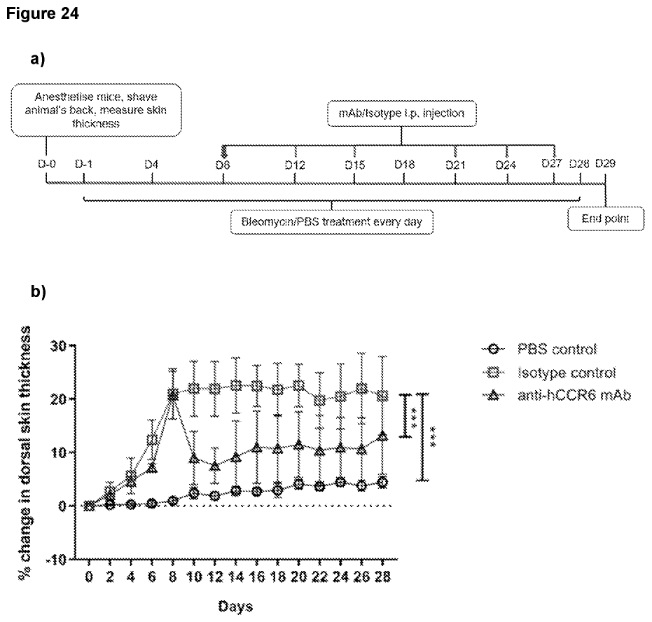

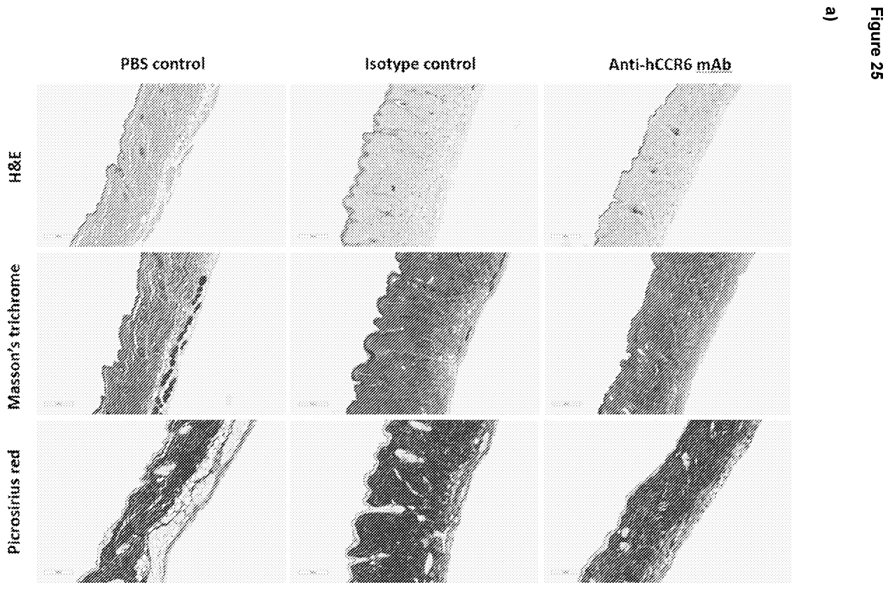

Bleomycin (BLM) (Sigma) was diluted to 200 μg/ml with PBS. 100 μl bleomycin or PBS (vehicle) were injected subcutaneously into a single location on the shaved back of mice once daily for 28 days. Mice were treated subsequently with i.p. injections of anti-human CCR6 mAb (hAB6, as herein described) at 5 mg/kg, 3 times a week from day 8 until day 27. Control mice were treated by i.p. injections of Isotype control or PBS. A schematic of the experimental protocol is shown in FIG. 24 a).

Increased thickness is typically observed following treatment with bleomycin, indicative of scleroderma. This thickness increases and persists when isotype control antibody is administered. However, as shown in FIG. 24 b), dorsal skin thickness was significantly reduced in mice that received anti-human CCR6 antibody following bleomycin injection.

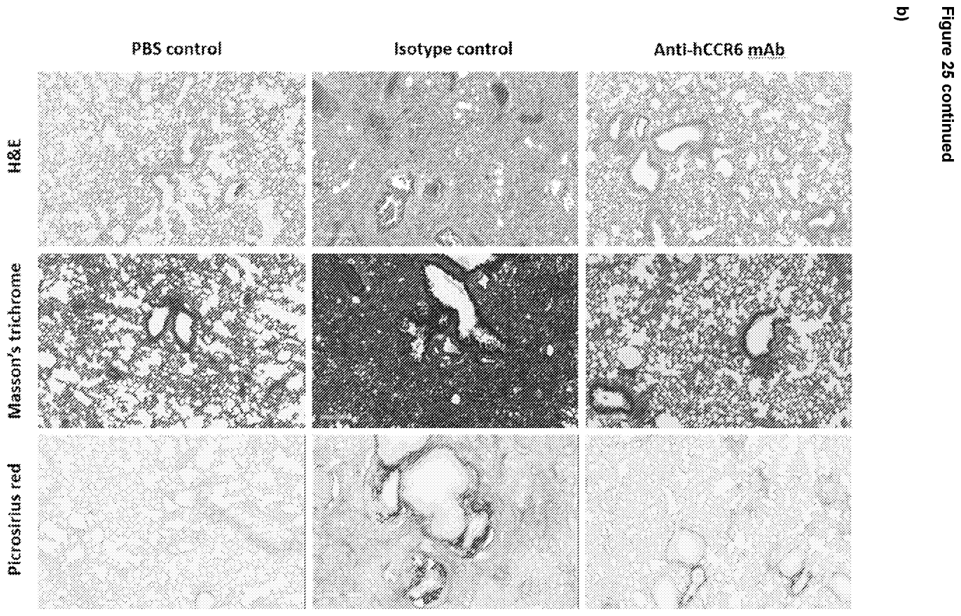

FIG. 25 shows the results of histological evaluation from the mice. FIG. 25 a) shows H&E, Mason's trichome and Picosirus red staining of skin tissue and FIG. 25 b) shows the same staining of lung tissue.

These results indicate that the anti-CCR6 depleting antibodies of the invention are useful for treating and reducing symptoms of scleroderma, including systemic scleroderma.

1. An antigen binding protein that comprises an antigen binding domain that binds to CCR6, wherein preferably the antigen binding protein inhibits binding of MIP-3α to CCR6.

2. The antigen binding protein of claim 1, wherein the antigen binding domain binds to a peptide, wherein the peptide:
consists of the sequence of SEQ ID NO: 2; or
consists of a sequence within the sequence of SEQ ID NO: 2, said peptide being useful as an immunogen to generate an antibody that is capable of binding to a CCR6.

3. The antigen binding protein of claim 1, wherein the antigen binding domain binds to:
a peptide consisting of amino acids 1 to 28 of CCR6, and/or
a peptide consisting of amino acids 18 to 46 of CCR6

4. The antigen binding protein of any one of claims 1 to 3, wherein the CCR6 is human CCR6.

5. The antigen binding protein of any one of claims 1 to 3, wherein the antigen binding protein comprises:
FR1-CDR1-FR2-CDR2-FR3-CDR3-FR4, and
FR1a-CDR1a-FR2a-CDR2a-FR3a-CDR3a-FR4a,
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the sequence of any of the framework regions or complementarity determining regions are as described herein.

6. The antigen binding protein of any one of claims 1 to 5, wherein the antigen binding protein comprises:
FR1-CDR1-FR2-CDR2-FR3-CDR3-FR4, and
FR1a-CDR1a-FR2a-CDR2a-FR3a-CDR3a-FR4a,
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the sequence of any of the complementarity determining regions have an amino acid sequence as described in Table 1 or 2 herein and wherein preferably, the framework regions have an amino acid sequence as described in Table 3 or 4.

7. The antigen binding protein of any one of claims 1 to 6, wherein the antigen binding protein comprises:
FR1-CDR1-FR2-CDR2-FR3-CDR3-FR4-linker-FR1a-CDR1a-FR2a-CDR2a-FR3a-CDR3a-FR4a.

8. The antigen binding protein of claim 7, wherein the linker is a chemical, one or more amino acids, or a disulphide bond formed between two cysteine residues.

9. The antigen binding protein of any one of claims 5 to 8, wherein the antigen binding domain binds to or specifically binds to CCR6 and wherein the antigen binding domain comprises at least one of:
(i) a VH comprising a complementarity determining region (CDR) 1 comprising a sequence at least about 60%, at least 70%, at least 75%, at least 80%, at least 85%, at least 90%, at least 92%, at least 95%, at least 97%, at least 99% identical to the sequence set forth in SEQ ID NO:11, a CDR2 comprising a sequence at least about 60%, at least 70%, at least 75%, at least 80%, at least 85%, at least 90%, at least 92%, at least 95%, at least 97%, at least 99% identical to the sequence set in SEQ ID NO:12 and a CDR3 comprising a sequence at least about 60%, at least 70%, at least 75%, at least 80%, at least 85%, at least 90%, at least 92%, at least 95%, at least 97%, at least 99% identical to the sequence set forth in SEQ ID NO: 13;
(ii) a VH comprising a sequence at least about 80%, at least 85%, at least 90%, at least 92%, at least 95% at least 96%, at least 97%, at least 98%, at least 99% identical to the sequence set forth in SEQ ID NO: 88;
(iii) a VL comprising a CDR1 comprising a sequence at least about 60%, at least 70%, at least 75%, at least 80%, at least 85%, at least 90%, at least 92%, at least 95%, at least 97%, at least 99% identical to the sequence set forth in SEQ ID NO: 17, a CDR2 comprising a sequence at least about 65%, at least about 66%, at least 80%, at least 85%, at least 90%, at least 92%, at least 95%, at least 97%, at least 99% identical to the sequence set forth in SEQ ID NO: 18 and a CDR3 comprising a sequence at least about 60%, at least about 70%, at least about 75%, at least 80%, at least 85%, at least 90%, at least 92%, at least 95%, at least 97%, at least 99% identical to the sequence set forth in SEQ ID NO: 19;
(iv) a VL comprising a sequence at least about at least about 80%, at least 85%, at least 90%, at least 92%, at least 95% at least 96%, at least 97%, at least 98%, at least 99% identical to the sequence set forth in SEQ ID NO: 89;
(v) a VH comprising a CDR1 comprising the sequence set forth in SEQ ID NO: 11, a CDR2 comprising the sequence set forth in SEQ ID NO: 12 and a CDR3 comprising the sequence set forth in SEQ ID NO: 13;
(vi) a VH comprising the sequence set forth in SEQ ID NO: 88;
(vii) a VL comprising a CDR1 comprising the sequence set SEQ ID NO: 17, a CDR2 comprising the sequence set forth in SEQ ID NO: 18 and a CDR3 comprising the sequence set forth in SEQ ID NO: 19;
(viii) a VL comprising the sequence set forth in SEQ ID NO: 89;
(ix) a VH comprising a CDR1 comprising the sequence set forth in SEQ ID NO: 11, a CDR2 comprising the sequence set forth in SEQ ID NO: 12 and a CDR3 comprising a sequence set forth in SEQ ID NO: 13; and a VL comprising a CDR1 comprising the sequence set SEQ ID NO: 17, a CDR2 comprising the sequence set forth in SEQ ID NO: 18 and a CDR3 comprising the sequence set forth in SEQ ID NO: 19; or
(x) a VH comprising the sequence set forth in SEQ ID NO: 88 and a VL comprising the sequence set forth in SEQ ID NO: 89.

10. The antigen binding protein of claim 9 wherein the antigen binding protein further comprises at least one of:
(i) a VH comprising a framework region (FR) 1 comprising a sequence at least about 80%, at least 85%, at least 90%, at least 92%, at least 95%, at least 97%, at least 99% identical to the sequence set forth in SEQ ID NO:80, a FR2 comprising a sequence at least about 80%, at least 85%, at least 90%, at least 92%, at least 95%, at least 97%, at least 99% identical to the sequence set in SEQ ID NO:81, a FR3 comprising a sequence at least about 80%, at least 85%, at least 90%, at least 92%, at least 95%, at least 97%, at least 99% identical to the sequence set forth in SEQ ID NO: 82, and a FR4 comprising a sequence at least about 80%, at least 85%, at least 90%, at least 92%, at least 95%, at least 97%, at least 99% identical to the sequence set forth in SEQ ID NO: 83;
(ii) a VL comprising a FR1 comprising a sequence at least about 80%, at least 85%, at least 90%, at least 92%, at least 95%, at least 97%, at least 99% identical to the sequence set forth in SEQ ID NO: 84, a FR2 comprising a sequence at least about 80%, at least 85%, at least 90%, at least 92%, at least 95%, at least 97%, at least 99% identical to the sequence set forth in SEQ ID NO: 85, a FR3 comprising a sequence at least about 80%, at least 85%, at least 90%, at least 92%, at least 95%, at least 97%, at least 99% identical to the sequence set forth in SEQ ID NO: 86, and a FR4 comprising a sequence at least about 80%, at least 85%, at least 90%, at least 92%, at least 95%, at least 97%, at least 99% identical to the sequence set forth in SEQ ID NO: 87;
(iii) a VH comprising a FR1 comprising the sequence set forth in SEQ ID NO: 80, a FR2 comprising the sequence set forth in SEQ ID NO: 81, a FR3 comprising the sequence set forth in SEQ ID NO: 82, and a FR4 comprising the sequence set forth in SEQ ID NO: 83;
(iv) a VL comprising a FR1 comprising the sequence set forth in SEQ ID NO: 84, a FR2 comprising the sequence set forth in SEQ ID NO: 85, a FR3 comprising the sequence set forth in SEQ ID NO: 86, and a FR4 comprising the sequence set forth in SEQ ID NO: 87; or
(v) a VH comprising a FR1 comprising the sequence set forth in SEQ ID NO: 80, a FR2 comprising the sequence set forth in SEQ ID NO: 81, a FR3 comprising a sequence set forth in SEQ ID NO: 82, and a FR4 comprising the sequence set forth in SEQ ID NO: 83; and a VL comprising a FR1 comprising the sequence set forth in SEQ ID NO: 84, a FR2 comprising the sequence set forth in SEQ ID NO: 85, a FR3 comprising the sequence set forth in SEQ ID NO: 86, and a FR4 comprising the sequence set forth in SEQ ID NO: 87.

11. The antigen binding protein of any one of claims 1 to 10, wherein the antigen binding protein comprises, consists essentially of or consists of the amino acid sequence as set forth in SEQ ID NOs: 88 and 89.

12. The antigen binding protein of any one of claims 5 to 8, wherein the antigen binding domain binds to or specifically binds to CCR6 and wherein the antigen binding domain comprises at least one of:
(i) a VH comprising a complementarity determining region (CDR) 1 comprising a sequence at least about 60%, at least 70%, at least 75%, at least 80%, at least 85%, at least 90%, at least 92%, at least 95%, at least 97%, at least 99% identical to the sequence set forth in SEQ ID NO: 11, a CDR2 comprising a sequence at least about 60%, at least 70%, at least 75%, at least 80%, at least 85%, at least 90%, at least 92%, at least 95%, at least 97%, at least 99% identical to the sequence set in SEQ ID NO: 12 and a CDR3 comprising a sequence at least about 60%, at least 70%, at least 75%, at least 80%, at least 85%, at least 90%, at least 92%, at least 95%, at least 97%, at least 99% identical to the sequence set forth in SEQ ID NO: 5;
(ii) a VH comprising a sequence at least about 80%, at least 85%, at least 90%, at least 92%, at least 95% at least 96%, at least 97%, at least 98%, at least 99% identical to the sequence set forth in SEQ ID NO: 96 or 97;
(iii) a VL comprising a CDR1 comprising a sequence at least about 60%, at least 70%, at least 75%, at least 80%, at least 85%, at least 90%, at least 92%, at least 95%, at least 97%, at least 99% identical to the sequence set forth in SEQ ID NO: 17, a CDR2 comprising a sequence at least about 65%, at least about 66%, at least 80%, at least 85%, at least 90%, at least 92%, at least 95%, at least 97%, at least 99% identical to the sequence set forth in SEQ ID NO: 18 and a CDR3 comprising a sequence at least about 60%, at least about 70%, at least about 75%, at least 80%, at least 85%, at least 90%, at least 92%, at least 95%, at least 97%, at least 99% identical to the sequence set forth in SEQ ID NO: 19;
(iv) a VL comprising a sequence at least about at least about 80%, at least 85%, at least 90%, at least 92%, at least 95% at least 96%, at least 97%, at least 98%, at least 99% identical to the sequence set forth in SEQ ID NO: 89;
(v) a VH comprising a CDR1 comprising the sequence set forth in SEQ ID NO: 11, a CDR2 comprising the sequence set forth in SEQ ID NO: 12 and a CDR3 comprising the sequence set forth in SEQ ID NO: 5;
(vi) a VH comprising the sequence set forth in SEQ ID NO: 96 or 97;
(vii) a VL comprising a CDR1 comprising the sequence set SEQ ID NO: 17, a CDR2 comprising the sequence set forth in SEQ ID NO: 18 and a CDR3 comprising the sequence set forth in SEQ ID NO: 19;
(viii) a VL comprising the sequence set forth in SEQ ID NO: 89;
(ix) a VH comprising a CDR1 comprising a sequence set forth in SEQ ID NO: 11, a CDR2 comprising the sequence set forth in SEQ ID NO: 12 and a CDR3 comprising the sequence set forth in SEQ ID NO: 5; and a VL comprising a CDR1 comprising the sequence set SEQ ID NO: 17, a CDR2 comprising the sequence set forth in SEQ ID NO: 18 and a CDR3 comprising the sequence set forth in SEQ ID NO: 19; or
(x) a VH comprising the sequence set forth in SEQ ID NO: 96 or 97 and a VL comprising the sequence set forth in SEQ ID NO: 89.

13. The antigen binding protein of any one of claims 5 to 8, wherein the antigen binding domain binds to or specifically binds to CCR6 and wherein the antigen binding domain comprises at least one of
(i) a VH comprising a complementarity determining region (CDR) 1 comprising a sequence at least about 60%, at least 70%, at least 75%, at least 80%, at least 85%, at least 90%, at least 92%, at least 95%, at least 97%, at least 99% identical to the sequence set forth in SEQ ID NO:11, a CDR2 comprising a sequence at least about 60%, at least 70%, at least 75%, at least 80%, at least 85%, at least 90%, at least 92%, at least 95%, at least 97%, at least 99% identical to the sequence set in SEQ ID NO:12 and a CDR3 comprising a sequence at least about 60%, at least 70%, at least 75%, at least 80%, at least 85%, at least 90%, at least 92%, at least 95%, at least 97%, at least 99% identical to the sequence set forth in SEQ ID NO: 13;
(ii) a VH comprising a sequence at least about 80%, at least 85%, at least 90%, at least 92%, at least 95% at least 96%, at least 97%, at least 98%, at least 99% identical to the sequence set forth in SEQ ID NO: 88;
(iii) a VL comprising a CDR1 comprising a sequence at least about 60%, at least 70%, at least 75%, at least 80%, at least 85%, at least 90%, at least 92%, at least 95%, at least 97%, at least 99% identical to the sequence set forth in SEQ ID NO: 94, a CDR2 comprising a sequence at least about 65%, at least about 66%, at least 80%, at least 85%, at least 90%, at least 92%, at least 95%, at least 97%, at least 99% identical to the sequence set forth in SEQ ID NO: 18 and a CDR3 comprising a sequence at least about 60%, at least about 70%, at least about 75%, at least 80%, at least 85%, at least 90%, at least 92%, at least 95%, at least 97%, at least 99% identical to the sequence set forth in SEQ ID NO: 19;
(iv) a VL comprising a sequence at least about at least about 80%, at least 85%, at least 90%, at least 92%, at least 95% at least 96%, at least 97%, at least 98%, at least 99% identical to the sequence set forth in SEQ ID NO: 98;
(v) a VH comprising a CDR1 comprising the sequence set forth in SEQ ID NO: 11, a CDR2 comprising a sequence set forth in SEQ ID NO: 12 and a CDR3 comprising the sequence set forth in SEQ ID NO: 13;
(vi) a VH comprising the sequence set forth in SEQ ID NO: 88;
(vii) a VL comprising a CDR1 comprising a sequence set SEQ ID NO: 94, a CDR2 comprising the sequence set forth in SEQ ID NO: 18 and a CDR3 comprising the sequence set forth in SEQ ID NO: 19;
(viii) a VL comprising a sequence set forth in SEQ ID NO: 98;
(ix) a VH comprising a CDR1 comprising the sequence set forth in SEQ ID NO: 11, a CDR2 comprising the sequence set forth in SEQ ID NO: 12 and a CDR3 comprising the sequence set forth in SEQ ID NO: 13; and a VL comprising a CDR1 comprising the sequence set SEQ ID NO: 94, a CDR2 comprising the sequence set forth in SEQ ID NO: 18 and a CDR3 comprising the sequence set forth in SEQ ID NO: 19; or
(x) a VH comprising the sequence set forth in SEQ ID NO: 88 and a VL comprising the sequence set forth in SEQ ID NO: 98.

14. The antigen binding protein of any one of claims 5 to 8, wherein the antigen binding domain binds to or specifically binds to CCR6 and wherein the antigen binding domain comprises at least one of
(i) a VH comprising a complementarity determining region (CDR) 1 comprising a sequence at least about 60%, at least 70%, at least 75%, at least 80%, at least 85%, at least 90%, at least 92%, at least 95%, at least 97%, at least 99% identical to the sequence set forth in SEQ ID NO: 11, a CDR2 comprising a sequence at least about 60%, at least 70%, at least 75%, at least 80%, at least 85%, at least 90%, at least 92%, at least 95%, at least 97%, at least 99% identical to the sequence set in SEQ ID NO: 12 and a CDR3 comprising a sequence at least about 60%, at least 70%, at least 75%, at least 80%, at least 85%, at least 90%, at least 92%, at least 95%, at least 97%, at least 99% identical to the sequence set forth in SEQ ID NO: 5;
(ii) a VH comprising a sequence at least about 80%, at least 85%, at least 90%, at least 92%, at least 95% at least 96%, at least 97%, at least 98%, at least 99% identical to the sequence set forth in SEQ ID NO: 96 or 97;
(iii) a VL comprising a CDR1 comprising a sequence at least about 60%, at least 70%, at least 75%, at least 80%, at least 85%, at least 90%, at least 92%, at least 95%, at least 97%, at least 99% identical to the sequence set forth in SEQ ID NO: 94, a CDR2 comprising a sequence at least about 65%, at least about 66%, at least 80%, at least 85%, at least 90%, at least 92%, at least 95%, at least 97%, at least 99% identical to the sequence set forth in SEQ ID NO: 18 and a CDR3 comprising a sequence at least about 60%, at least about 70%, at least about 75%, at least 80%, at least 85%, at least 90%, at least 92%, at least 95%, at least 97%, at least 99% identical to the sequence set forth in SEQ ID NO: 19;
(iv) a VL comprising a sequence at least about at least about 80%, at least 85%, at least 90%, at least 92%, at least 95% at least 96%, at least 97%, at least 98%, at least 99% identical to the sequence set forth in SEQ ID NO: 98;
(v) a VH comprising a CDR1 comprising the sequence set forth in SEQ ID NO: 11, a CDR2 comprising a sequence set forth in SEQ ID NO: 12 and a CDR3 comprising the sequence set forth in SEQ ID NO: 5;
(vi) a VH comprising the sequence set forth in SEQ ID NO: 96 or 97;
(vii) a VL comprising a CDR1 comprising a sequence set SEQ ID NO: 94, a CDR2 comprising the sequence set forth in SEQ ID NO: 18 and a CDR3 comprising the sequence set forth in SEQ ID NO: 19;
(viii) a VL comprising a sequence set forth in SEQ ID NO: 98;
(ix) a VH comprising a CDR1 comprising the sequence set forth in SEQ ID NO: 11, a CDR2 comprising the sequence set forth in SEQ ID NO: 12 and a CDR3 comprising the sequence set forth in SEQ ID NO: 5; and a VL comprising a CDR1 comprising the sequence set SEQ ID NO: 94, a CDR2 comprising the sequence set forth in SEQ ID NO: 18 and a CDR3 comprising the sequence set forth in SEQ ID NO: 19; or
(x) a VH comprising the sequence set forth in SEQ ID NO: 96 or 97 and a VL comprising the sequence set forth in SEQ ID NO: 98.

15. The antigen binding protein of any one of claims 5 to 8, wherein
CDR1 has a sequence selected from the group consisting of:
(G/E)(F/Y)(T/S/P)F(S/K)(D/S)(Y/F)(Y/G),
GF(S/T/P)FSDYY,
(SEQ ID NO: 3)
GFTFSDYY,
(SEQ ID NO: 6)
GFSFSDYY,
(SEQ ID NO: 11)
GFPFSDYY
and
(SEQ ID NO: 14)
EYTFKSFG;
CDR2 has a sequence selected from the group consisting of:
I(T/Y)(N/P)(G/R)(D/G/A/V/S)G(R/N)T,
ITNG(D/G/A/V)GRT,
(SEQ ID NO: 4)
ITNGDGRT,
(SEQ ID NO: 7)
ITNGGGRT,
(SEQ ID NO: 9)
ITNGAGRT,
(SEQ ID NO: 12)
ITNGVGRT
and
(SEQ ID NO: 15)
IYPRSGNT;
CDR3 has a sequence selected from the group consisting of:
(T/A)(S/R)(P/S)P(L/Y)(G/D)G(A/-)(W/Y)F(G/A/D)Y,
(A/T)SPPLGGAWF(G/A)Y,
(SEQ ID NO: 5)
TSPPLGGAWFGY,
(SEQ ID NO: 8)
ASPPLGGAWFGY,
(SEQ ID NO: 10)
ASPPLGGAWFAY,
(SEQ ID NO: 13)
TSPPLGGAWFAY
and
(SEQ ID NO: 16)
ARSPYDGYFDY;
CDR1a has a sequence selected from the group consisting of:
QS(I/L)(V/L)H(S/I)NGNTY,
QS(I/L)VHSNGNTY,
(SEQ ID NO: 17)
QSIVHSNGNTY,
(SEQ ID NO: 20)
QSLVHSNGNTY
and
(SEQ ID NO: 21)
QSLLHINGNTY;
CDR2a has a sequence selected from the group consisting of: (K/R)VS, RVS (SEQ ID NO: 22) and KVS (SEQ ID NO: 18); and
CDR3a has a sequence selected from the group consisting of:
(F/S)Q(G/S)(S/T)HVP(L/R)T,
(SEQ ID NO: 19)
FQGSHVPLT
and
(SEQ ID NO: 23)
SQSTHVPRT;

16. The antigen binding protein of claim 15 wherein:
FR1 has the sequence selected from the groups consisting of:
(SEQ ID NO: 24)
EVNLVESGGGLVQPGGSLKLSCAAS,
(SEQ ID NO: 25)
EVNLVESGGGLVQPGGSLKLSCEAS,
(SEQ ID NO: 26)
EVKLVESGGGLVQPGGSLKLSCAAS,
(SEQ ID NO: 27)
QDQLQQSGVALARPGASVKLSCKAS,
(SEQ ID NO: 90)
EVNLVESGGGLVQPGGSLILSCEAS
and
(SEQ ID NO: 80)
EVQLVESGGGLVQPGGSLRLSCAAS;
FR2 has the sequence selected from the group consisting of:
(SEQ ID NO: 28)
MYWVRQTPEKRLEWVTY,
(SEQ ID NO: 29)
LYWVRQTPEKRLEWVTY,
(SEQ ID NO: 30)
LYWVRQTPEKRLEWVAY,
(SEQ ID NO: 31)
LGWVKQRPGQGLEWIGE
and
(SEQ ID NO: 81)
LYWVRQAPGKGLEWVAY;
FR3 has the sequence selected from the group consisting of:
(SEQ ID NO: 32)
YYSDTVRGRFTISRDNAKNTLYLQMSRLKSEDTAMYYC,
(SEQ ID NO: 33)
YYSDTIRGRFTISRDNARNTLYLQMSRLKSEDTAMYYC,
(SEQ ID NO: 34)
YYSDSVKGRFTISRDNAKNTLYLQMSRLKSEDTSMYYC,
(SEQ ID NO: 35)
YYNEKVKGKVRLTADKSSNSVYMEFRSLTSEDSAVYFC,
(SEQ ID NO: 91)
YYSDAIRGRFTISRDNARNTLYLQMSRLKSEDTAMYYC
and
(SEQ ID NO: 82)
YYSDSVKGRFTISRDNAKNTLYLQMNSLRDEDTAVYYC;
FR4 has the sequence: WGQGTLVTVS (SEQ ID NO: 36) or WGQGTTLTVS (SEQ ID NO: 37);
FR1a has the sequence selected from the group consisting of:
(SEQ ID NO: 38)
DVLMTQTPLSLPVSLGDQASISCRSS,
(SEQ ID NO: 39)
DVSMTQTPLSLPVSLGDQASISCRSS,
(SEQ ID NO: 40)
DVVMTHSPLSLPVSLGDQASISCRSS
and
(SEQ ID NO: 84)
DIVMTQSPLSLPVTPGEPASISCRSS;
FR2a has the sequence: LEWYLQKPGQSPKLLIY (SEQ ID NO: 41), LHWYLQKPGQSPKLLIY (SEQ ID NO: 42) or LEWYLQKPGQSPRLLIY (SEQ ID NO: 85);
FR3a has the sequence selected from the group consisting of:
(SEQ ID NO: 43)
KRFSGVPDRFSGSGSGTDFTLKISRVEAEDLGVYYC,
(SEQ ID NO: 44)
KRFSGVPDRFSGSGSGTDFTLKISRVGAEDLGVYYC,
(SEQ ID NO: 45)
NRLSGVPDRFSGSGSGTDFTLKISRVEAEDLGVYFC
and
(SEQ ID NO: 86)
KRFSGVPDRFSGSGSGTDFTLKISRVEAEDVGVYYC;
and
FR4a has the sequence: FGAGTKLELKR (SEQ ID NO: 46), FGGGTKLEIKR (SEQ ID NO: 47) or FGQGTKLEIR (SEQ ID NO: 87).

17. The antigen binding protein of any one of claims 1 to 5, wherein the antigen binding protein comprises, consists essentially of or consists of the amino acid sequence as set forth in any one of SEQ ID NOs: 48 to 59, 88, 89, 92, 93, and 96 to 98.

18. The antigen binding protein of any one of claims 5 to 8, wherein the antigen binding domain binds to or specifically binds to CCR6 and wherein the antigen binding domain comprises at least one of:
(i) a VH comprising a complementarity determining region (CDR) 1 comprising a sequence at least about 60%, at least 70%, at least 75%, at least 80%, at least 85%, at least 90%, at least 92%, at least 95%, at least 97%, at least 99% identical to the sequence set forth in SEQ ID NO:3, a CDR2 comprising a sequence at least about 60%, at least 70%, at least 75%, at least 80%, at least 85%, at least 90%, at least 92%, at least 95%, at least 97%, at least 99% identical to the sequence set in SEQ ID NO:4 and a CDR3 comprising a sequence at least about 60%, at least 70%, at least 75%, at least 80%, at least 85%, at least 90%, at least 92%, at least 95%, at least 97%, at least 99% identical to the sequence set forth in SEQ ID NO: 5;
(ii) a VH comprising a sequence at least about 80%, at least 85%, at least 90%, at least 92%, at least 95% at least 96%, at least 97%, at least 98%, at least 99% identical to a sequence set forth in SEQ ID NO: 48;
(iii) a VL comprising a CDR1 comprising a sequence at least about 60%, at least 70%, at least 75%, at least 80%, at least 85%, at least 90%, at least 92%, at least 95%, at least 97%, at least 99% identical to the sequence set forth in SEQ ID NO: 17, a CDR2 comprising a sequence at least about 65%, at least about 66%, at least 80%, at least 85%, at least 90%, at least 92%, at least 95%, at least 97%, at least 99% identical to the sequence set forth in SEQ ID NO: 18 and a CDR3 comprising a sequence at least about 60%, at least about 70%, at least about 75%, at least 80%, at least 85%, at least 90%, at least 92%, at least 95%, at least 97%, at least 99% identical to the sequence set forth in SEQ ID NO: 19;
(iv) a VL comprising a sequence at least about at least about 80%, at least 85%, at least 90%, at least 92%, at least 95% at least 96%, at least 97%, at least 98%, at least 99% identical to the sequence set forth in SEQ ID NO: 55;
(v) a VH comprising a CDR1 comprising the sequence as set forth in SEQ ID NO: 3, a CDR2 comprising the sequence set forth in SEQ ID NO: 4 and a CDR3 comprising the sequence set forth in SEQ ID NO: 5;
(vi) a VH comprising a sequence set forth in SEQ ID NO: 48;
(vii) a VL comprising a CDR1 comprising a sequence set SEQ ID NO: 17, a CDR2 comprising the sequence set forth in SEQ ID NO: 18 and a CDR3 comprising the sequence set forth in SEQ ID NO: 19;
(viii) a VL comprising the sequence set forth in SEQ ID NO: 55;
(ix) a VH comprising a CDR1 comprising the sequence set forth in SEQ ID NO: 3, a CDR2 comprising a sequence set forth between in SEQ ID NO: 4 and a CDR3 comprising the sequence set forth in SEQ ID NO: 5; and a VL comprising a CDR1 comprising a sequence set SEQ ID NO: 17, a CDR2 comprising the sequence set forth in SEQ ID NO: 18 and a CDR3 comprising the sequence set forth in SEQ ID NO: 19; or
(x) a VH comprising a sequence set forth in SEQ ID NO: 48 and a VL comprising the sequence set forth in SEQ ID NO: 55.

19. The antigen binding protein of any one of claims 5 to 8, wherein the antigen binding domain binds to or specifically binds to CCR6 and wherein the antigen binding domain comprises at least one of:
(i) a VH comprising a complementarity determining region (CDR) 1 comprising a sequence at least about 60%, at least 70%, at least 75%, at least 80%, at least 85%, at least 90%, at least 92%, at least 95%, at least 97%, at least 99% identical to the sequence set forth in SEQ ID NO:6, a CDR2 comprising a sequence at least about 60%, at least 70%, at least 75%, at least 80%, at least 85%, at least 90%, at least 92%, at least 95%, at least 97%, at least 99% identical to the sequence set in SEQ ID NO:7 and a CDR3 comprising a sequence at least about 60%, at least 70%, at least 75%, at least 80%, at least 85%, at least 90%, at least 92%, at least 95%, at least 97%, at least 99% identical to the sequence set forth in SEQ ID NO: 5;
(ii) a VH comprising a sequence at least about 80%, at least 85%, at least 90%, at least 92%, at least 95% at least 96%, at least 97%, at least 98%, at least 99% identical to the sequence set forth in SEQ ID NO: 49;
(iii) a VL comprising a CDR1 comprising a sequence at least about 60%, at least 70%, at least 75%, at least 80%, at least 85%, at least 90%, at least 92%, at least 95%, at least 97%, at least 99% identical to the sequence set forth in SEQ ID NO: 20, a CDR2 comprising a sequence at least about 65%, at least about 66%, at least 80%, at least 85%, at least 90%, at least 92%, at least 95%, at least 97%, at least 99% identical to the sequence set forth in SEQ ID NO: 18 and a CDR3 comprising a sequence at least about 60%, at least about 70%, at least about 75%, at least 80%, at least 85%, at least 90%, at least 92%, at least 95%, at least 97%, at least 99% identical to the sequence set forth in SEQ ID NO: 19;
(iv) a VL comprising a sequence at least about at least about 80%, at least 85%, at least 90%, at least 92%, at least 95% at least 96%, at least 97%, at least 98%, at least 99% identical to the sequence set forth in SEQ ID NO: 56;
(v) a VH comprising a CDR1 comprising a sequence set forth in SEQ ID NO: 6, a CDR2 comprising a sequence set forth in SEQ ID NO: 7 and a CDR3 comprising the sequence set forth in SEQ ID NO: 5;
(vi) a VH comprising a sequence set forth in SEQ ID NO: 49;
(vii) a VL comprising a CDR1 comprising the sequence set SEQ ID NO: 20, a CDR2 comprising the sequence set forth in SEQ ID NO: 18 and a CDR3 comprising the sequence set forth in SEQ ID NO: 19;
(viii) a VL comprising a sequence set forth in SEQ ID NO: 56;
(ix) a VH comprising a CDR1 comprising the sequence set forth in SEQ ID NO: 6, a CDR2 comprising the sequence set forth in SEQ ID NO: 7 and a CDR3 comprising the sequence set forth in SEQ ID NO: 5; and a VL comprising a CDR1 comprising the sequence set SEQ ID NO: 20, a CDR2 comprising the sequence set forth in SEQ ID NO: 18 and a CDR3 comprising the sequence set forth in SEQ ID NO: 19; or
(x) a VH comprising the sequence set forth in SEQ ID NO: 49 and a VL comprising the sequence set forth in SEQ ID NO: 56.

20. The antigen binding protein of any one of claims 5 to 8, wherein the antigen binding domain binds to or specifically binds to CCR6 and wherein the antigen binding domain comprises at least one of:
(i) a VH comprising a complementarity determining region (CDR) 1 comprising a sequence at least about 60%, at least 70%, at least 75%, at least 80%, at least 85%, at least 90%, at least 92%, at least 95%, at least 97%, at least 99% identical to the sequence set forth in SEQ ID NO:3, a CDR2 comprising a sequence at least about 60%, at least 70%, at least 75%, at least 80%, at least 85%, at least 90%, at least 92%, at least 95%, at least 97%, at least 99% identical to the sequence set in SEQ ID NO:7 and a CDR3 comprising a sequence at least about 60%, at least 70%, at least 75%, at least 80%, at least 85%, at least 90%, at least 92%, at least 95%, at least 97%, at least 99% identical to the sequence set forth in SEQ ID NO: 8;
(ii) a VH comprising a sequence at least about 80%, at least 85%, at least 90%, at least 92%, at least 95% at least 96%, at least 97%, at least 98%, at least 99% identical to the sequence set forth in SEQ ID NO: 50;
(iii) a VL comprising a CDR1 comprising a sequence at least about 60%, at least 70%, at least 75%, at least 80%, at least 85%, at least 90%, at least 92%, at least 95%, at least 97%, at least 99% identical to the sequence set forth in SEQ ID NO: 17, a CDR2 comprising a sequence at least about 65%, at least about 66%, at least 80%, at least 85%, at least 90%, at least 92%, at least 95%, at least 97%, at least 99% identical to the sequence set forth in SEQ ID NO: 18 and a CDR3 comprising a sequence at least about 60%, at least about 70%, at least about 75%, at least 80%, at least 85%, at least 90%, at least 92%, at least 95%, at least 97%, at least 99% identical to the sequence set forth in SEQ ID NO: 19;
(iv) a VL comprising a sequence at least about at least about 80%, at least 85%, at least 90%, at least 92%, at least 95% at least 96%, at least 97%, at least 98%, at least 99% identical to a sequence set forth in SEQ ID NO: 57;
(v) a VH comprising a CDR1 comprising a sequence set forth in SEQ ID NO: 3, a CDR2 comprising the sequence set forth in SEQ ID NO: 7 and a CDR3 comprising the sequence set forth in SEQ ID NO: 8;
(vi) a VH comprising the sequence set forth in SEQ ID NO: 50;
(vii) a VL comprising a CDR1 comprising the sequence set SEQ ID NO: 17, a CDR2 comprising the sequence set forth in SEQ ID NO: 18 and a CDR3 comprising the sequence set forth in SEQ ID NO: 19;
(viii) a VL comprising the sequence set forth in SEQ ID NO: 57;
(ix) a VH comprising a CDR1 comprising the sequence set forth in SEQ ID NO: 3, a CDR2 the a sequence set forth between in SEQ ID NO: 7 and a CDR3 comprising the sequence set forth in SEQ ID NO: 8; and a VL comprising a CDR1 comprising the sequence set SEQ ID NO: 17, a CDR2 comprising the sequence set forth in SEQ ID NO: 18 and a CDR3 comprising a sequence set forth in SEQ ID NO: 19; or
(x) a VH comprising the sequence set forth in SEQ ID NO: 50 and a VL comprising the sequence set forth in SEQ ID NO: 57.

21. The antigen binding protein of any one of claims 5 to 8, wherein the antigen binding domain binds to or specifically binds to CCR6 and wherein the antigen binding domain comprises at least one of:
(i) a VH comprising a complementarity determining region (CDR) 1 comprising a sequence at least about 60%, at least 70%, at least 75%, at least 80%, at least 85%, at least 90%, at least 92%, at least 95%, at least 97%, at least 99% identical to the sequence set forth in SEQ ID NO:3, a CDR2 comprising a sequence at least about 60%, at least 70%, at least 75%, at least 80%, at least 85%, at least 90%, at least 92%, at least 95%, at least 97%, at least 99% identical to the sequence set in SEQ ID NO:7 and a CDR3 comprising a sequence at least about 60%, at least 70%, at least 75%, at least 80%, at least 85%, at least 90%, at least 92%, at least 95%, at least 97%, at least 99% identical to the sequence set forth in SEQ ID NO: 8;
(ii) a VH comprising a sequence at least about 80%, at least 85%, at least 90%, at least 92%, at least 95% at least 96%, at least 97%, at least 98%, at least 99% identical to the sequence set forth in SEQ ID NO: 51;
(iii) a VL comprising a CDR1 comprising a sequence at least about 60%, at least 70%, at least 75%, at least 80%, at least 85%, at least 90%, at least 92%, at least 95%, at least 97%, at least 99% identical to the sequence set forth in SEQ ID NO: 20, a CDR2 comprising a sequence at least about 65%, at least about 66%, at least 80%, at least 85%, at least 90%, at least 92%, at least 95%, at least 97%, at least 99% identical to the sequence set forth in SEQ ID NO: 18 and a CDR3 comprising a sequence at least about 60%, at least about 70%, at least about 75%, at least 80%, at least 85%, at least 90%, at least 92%, at least 95%, at least 97%, at least 99% identical to the sequence set forth in SEQ ID NO: 19;
(iv) a VL comprising a sequence at least about at least about 80%, at least 85%, at least 90%, at least 92%, at least 95% at least 96%, at least 97%, at least 98%, at least 99% identical to the sequence set forth in SEQ ID NO: 58;
(v) a VH comprising a CDR1 comprising a sequence set forth in SEQ ID NO: 3, a CDR2 comprising a sequence set forth between in SEQ ID NO: 7 and a CDR3 comprising the sequence set forth in SEQ ID NO: 8;
(vi) a VH comprising the sequence set forth in SEQ ID NO: 51;
(vii) a VL comprising a CDR1 comprising the sequence set SEQ ID NO: 20, a CDR2 comprising the sequence set forth in SEQ ID NO: 18 and a CDR3 comprising the sequence set forth in SEQ ID NO: 19;
(viii) a VL comprising the sequence set forth in SEQ ID NO: 58;
(ix) a VH comprising a CDR1 comprising the sequence set forth in SEQ ID NO: 3, a CDR2 comprising a sequence set forth between in SEQ ID NO: 7 and a CDR3 comprising the sequence set forth in SEQ ID NO: 8; and a VL comprising a CDR1 comprising the sequence set SEQ ID NO: 20, a CDR2 comprising a sequence set forth in SEQ ID NO: 18 and a CDR3 comprising the sequence set forth in SEQ ID NO: 19; or
(x) a VH comprising the sequence set forth in SEQ ID NO: 51 and a VL comprising the sequence set forth in SEQ ID NO: 58.

22. The antigen binding protein of any one of claims 5 to 8, wherein the antigen binding domain binds to or specifically binds to CCR6 and wherein the antigen binding domain comprises at least one of:
(i) a VH comprising a complementarity determining region (CDR) 1 comprising a sequence at least about 60%, at least 70%, at least 75%, at least 80%, at least 85%, at least 90%, at least 92%, at least 95%, at least 97%, at least 99% identical to the sequence set forth in SEQ ID NO:3, a CDR2 comprising a sequence at least about 60%, at least 70%, at least 75%, at least 80%, at least 85%, at least 90%, at least 92%, at least 95%, at least 97%, at least 99% identical to the sequence set in SEQ ID NO:9 and a CDR3 comprising a sequence at least about 60%, at least 70%, at least 75%, at least 80%, at least 85%, at least 90%, at least 92%, at least 95%, at least 97%, at least 99% identical to the sequence set forth in SEQ ID NO: 10;
(ii) a VH comprising a sequence at least about 80%, at least 85%, at least 90%, at least 92%, at least 95% at least 96%, at least 97%, at least 98%, at least 99% identical to the sequence set forth in SEQ ID NO: 52;
(iii) a VL comprising a CDR1 comprising a sequence at least about 60%, at least 70%, at least 75%, at least 80%, at least 85%, at least 90%, at least 92%, at least 95%, at least 97%, at least 99% identical to the sequence set forth in SEQ ID NO: 20, a CDR2 comprising a sequence at least about 65%, at least about 66%, at least 80%, at least 85%, at least 90%, at least 92%, at least 95%, at least 97%, at least 99% identical to the sequence set forth in SEQ ID NO: 18 and a CDR3 comprising a sequence at least about 60%, at least about 70%, at least about 75%, at least 80%, at least 85%, at least 90%, at least 92%, at least 95%, at least 97%, at least 99% identical to the sequence set forth in SEQ ID NO: 19;
(iv) a VL comprising a sequence at least about at least about 80%, at least 85%, at least 90%, at least 92%, at least 95% at least 96%, at least 97%, at least 98%, at least 99% identical to the sequence set forth in SEQ ID NO: 56;
(v) a VH comprising a CDR1 comprising the sequence set forth in SEQ ID NO: 3, a CDR2 comprising the sequence set forth between in SEQ ID NO: 9 and a CDR3 comprising the sequence set forth in SEQ ID NO: 10;
(vi) a VH comprising a sequence set forth in SEQ ID NO: 52;
(vii) a VL comprising a CDR1 comprising the sequence set SEQ ID NO: 20, a CDR2 comprising the sequence set forth in SEQ ID NO: 18 and a CDR3 comprising the sequence set forth in SEQ ID NO: 19;
(viii) a VL comprising the sequence set forth in SEQ ID NO: 56;
(ix) a VH comprising a CDR1 comprising the sequence set forth in SEQ ID NO: 3, a CDR2 comprising the sequence set forth between in SEQ ID NO: 9 and a CDR3 comprising the sequence set forth in SEQ ID NO: 10; and a VL comprising a CDR1 comprising the sequence set SEQ ID NO: 20, a CDR2 comprising a sequence set forth in SEQ ID NO: 18 and a CDR3 comprising the sequence set forth in SEQ ID NO: 19; or
(x) a VH comprising the sequence set forth in SEQ ID NO: 52 and a VL comprising a sequence set forth in SEQ ID NO: 56.

23. The antigen binding protein of any one of claims 5 to 8, wherein the antigen binding domain binds to or specifically binds to CCR6 and wherein the antigen binding domain comprises at least one of:
(i) a VH comprising a complementarity determining region (CDR) 1 comprising a sequence at least about 60%, at least 70%, at least 75%, at least 80%, at least 85%, at least 90%, at least 92%, at least 95%, at least 97%, at least 99% identical to the sequence set forth in SEQ ID NO:11, a CDR2 comprising a sequence at least about 60%, at least 70%, at least 75%, at least 80%, at least 85%, at least 90%, at least 92%, at least 95%, at least 97%, at least 99% identical to the sequence set in SEQ ID NO:12 and a CDR3 comprising a sequence at least about 60%, at least 70%, at least 75%, at least 80%, at least 85%, at least 90%, at least 92%, at least 95%, at least 97%, at least 99% identical to the sequence set forth in SEQ ID NO: 13;
(ii) a VH comprising a sequence at least about 80%, at least 85%, at least 90%, at least 92%, at least 95% at least 96%, at least 97%, at least 98%, at least 99% identical to the sequence set forth in SEQ ID NO: 53;
(iii) a VL comprising a CDR1 comprising a sequence at least about 60%, at least 70%, at least 75%, at least 80%, at least 85%, at least 90%, at least 92%, at least 95%, at least 97%, at least 99% identical to the sequence set forth in SEQ ID NO: 17, a CDR2 comprising a sequence at least about 65%, at least about 66%, at least 80%, at least 85%, at least 90%, at least 92%, at least 95%, at least 97%, at least 99% identical to the sequence set forth in SEQ ID NO: 18 and a CDR3 comprising a sequence at least about 60%, at least about 70%, at least about 75%, at least 80%, at least 85%, at least 90%, at least 92%, at least 95%, at least 97%, at least 99% identical to the sequence set forth in SEQ ID NO: 19;
(iv) a VL comprising a sequence at least about at least about 80%, at least 85%, at least 90%, at least 92%, at least 95% at least 96%, at least 97%, at least 98%, at least 99% identical to the sequence set forth in SEQ ID NO: 55;
(v) a VH comprising a CDR1 comprising the sequence set forth in SEQ ID NO: 11, a CDR2 comprising the sequence set forth in SEQ ID NO: 12 and a CDR3 comprising the sequence set forth in SEQ ID NO: 13;
(vi) a VH comprising the sequence set forth in SEQ ID NO: 53;
(vii) a VL comprising a CDR1 comprising the sequence set SEQ ID NO: 17, a CDR2 comprising the sequence set forth in SEQ ID NO: 18 and a CDR3 comprising the sequence set forth in SEQ ID NO: 19;
(viii) a VL comprising the sequence set forth in SEQ ID NO: 55;
(ix) a VH comprising a CDR1 comprising the sequence set forth in SEQ ID NO: 11, a CDR2 comprising the sequence set forth in SEQ ID NO: 12 and a CDR3 comprising a sequence set forth in SEQ ID NO: 13; and a VL comprising a CDR1 comprising the sequence set SEQ ID NO: 17, a CDR2 comprising the sequence set forth in SEQ ID NO: 18 and a CDR3 comprising the sequence set forth in SEQ ID NO: 19; or
(x) a VH comprising the sequence set forth in SEQ ID NO: 53 and a VL comprising the sequence set forth in SEQ ID NO: 55.

24. The antigen binding protein of claim 23 wherein the antigen binding protein further comprises at least one of:
(i) a VH comprising a framework region (FR) 1 comprising a sequence at least about 80%, at least 85%, at least 90%, at least 92%, at least 95%, at least 97%, at least 99% identical to the sequence set forth in SEQ ID NO: 26, a FR2 comprising a sequence at least about 80%, at least 85%, at least 90%, at least 92%, at least 95%, at least 97%, at least 99% identical to the sequence set in SEQ ID NO: 30, a FR3 comprising a sequence at least about 80%, at least 85%, at least 90%, at least 92%, at least 95%, at least 97%, at least 99% identical to the sequence set forth in SEQ ID NO: 34, and a FR4 comprising a sequence at least about 80%, at least 85%, at least 90%, at least 92%, at least 95%, at least 97%, at least 99% identical to the sequence set forth in SEQ ID NO: 36;
(ii) a VL comprising a FR1 comprising a sequence at least about 80%, at least 85%, at least 90%, at least 92%, at least 95%, at least 97%, at least 99% identical to the sequence set forth in SEQ ID NO: 38, a FR2 comprising a sequence at least about 80%, at least 85%, at least 90%, at least 92%, at least 95%, at least 97%, at least 99% identical to the sequence set forth in SEQ ID NO: 41, a FR3 comprising a sequence at least about 80%, at least 85%, at least 90%, at least 92%, at least 95%, at least 97%, at least 99% identical to the sequence set forth in SEQ ID NO: 43, and a FR4 comprising a sequence at least about 80%, at least 85%, at least 90%, at least 92%, at least 95%, at least 97%, at least 99% identical to the sequence set forth in SEQ ID NO: 46;
(iii) a VH comprising a FR1 comprising the sequence set forth in SEQ ID NO: 26, a FR2 comprising the sequence set forth in SEQ ID NO: 30, a FR3 comprising the sequence set forth in SEQ ID NO: 34, and a FR4 comprising the sequence set forth in SEQ ID NO: 36;
(iv) a VL comprising a FR1 comprising the sequence set forth in SEQ ID NO: 38, a FR2 comprising the sequence set forth in SEQ ID NO: 41, a FR3 comprising the sequence set forth in SEQ ID NO: 43, and a FR4 comprising the sequence set forth in SEQ ID NO: 46; or
(v) a VH comprising a FR1 comprising the sequence set forth in SEQ ID NO: 26, a FR2 comprising the sequence set forth in SEQ ID NO: 30, a FR3 comprising a sequence set forth in SEQ ID NO: 34, and a FR4 comprising the sequence set forth in SEQ ID NO: 36; and a VL comprising a FR1 comprising the sequence set forth in SEQ ID NO: 38, a FR2 comprising the sequence set forth in SEQ ID NO: 41, a FR3 comprising the sequence set forth in SEQ ID NO: 43, and a FR4 comprising the sequence set forth in SEQ ID NO: 46.

25. The antigen binding protein of any one of claims 5 to 8, wherein the antigen binding domain binds to or specifically binds to CCR6 and wherein the antigen binding domain comprises at least one of:
(i) a VH comprising a complementarity determining region (CDR) 1 comprising a sequence at least about 60%, at least 70%, at least 75%, at least 80%, at least 85%, at least 90%, at least 92%, at least 95%, at least 97%, at least 99% identical to the sequence set forth in SEQ ID NO:14, a CDR2 comprising a sequence at least about 60%, at least 70%, at least 75%, at least 80%, at least 85%, at least 90%, at least 92%, at least 95%, at least 97%, at least 99% identical to the sequence set in SEQ ID NO:15 and a CDR3 comprising a sequence at least about 60%, at least 70%, at least 75%, at least 80%, at least 85%, at least 90%, at least 92%, at least 95%, at least 97%, at least 99% identical the a sequence set forth in SEQ ID NO: 16;
(ii) a VH comprising a sequence at least about 80%, at least 85%, at least 90%, at least 92%, at least 95% at least 96%, at least 97%, at least 98%, at least 99% identical to the sequence set forth in SEQ ID NO: 54;
(iii) a VL comprising a CDR1 comprising a sequence at least about 60%, at least 70%, at least 75%, at least 80%, at least 85%, at least 90%, at least 92%, at least 95%, at least 97%, at least 99% identical to the sequence set forth in SEQ ID NO: 21, a CDR2 comprising a sequence at least about 65%, at least about 66%, at least 80%, at least 85%, at least 90%, at least 92%, at least 95%, at least 97%, at least 99% identical to the sequence set forth in SEQ ID NO: 22 and a CDR3 comprising a sequence at least about 60%, at least about 70%, at least about 75%, at least 80%, at least 85%, at least 90%, at least 92%, at least 95%, at least 97%, at least 99% identical to the sequence set forth in SEQ ID NO: 23;
(iv) a VL comprising a sequence at least about 80%, at least 85%, at least 90%, at least 92%, at least 95% at least 96%, at least 97%, at least 98%, at least 99% identical to the sequence set forth in SEQ ID NO: 59;
(v) a VH comprising a CDR1 comprising a the set forth in SEQ ID NO: 14, a CDR2 comprising the sequence set forth in SEQ ID NO: 15 and a CDR3 comprising the sequence set forth in SEQ ID NO: 16;
(vi) a VH comprising the sequence set forth in SEQ ID NO: 54;
(vii) a VL comprising a CDR1 comprising the sequence set SEQ ID NO: 21, a CDR2 comprising the sequence set forth in SEQ ID NO: 22 and a CDR3 comprising the sequence set forth in SEQ ID NO: 23;
(viii) a VL comprising a sequence set forth in SEQ ID NO: 59;
(ix) a VH comprising a CDR1 comprising a sequence set forth in SEQ ID NO:
14, a CDR2 comprising a sequence set forth between in SEQ ID NO: 15 and a CDR3 comprising the sequence set forth in SEQ ID NO: 16; and a VL comprising a CDR1 comprising the sequence set SEQ ID NO: 21, a CDR2 comprising a sequence set forth in SEQ ID NO: 22 and a CDR3 comprising the sequence set forth in SEQ ID NO: 23; or
(x) a VH comprising the sequence set forth in SEQ ID NO: 54 and a VL comprising the sequence set forth in SEQ ID NO: 59.

26. The antigen binding protein of any one of claims 5 to 8, wherein the antigen binding domain binds to or specifically binds to CCR6 and wherein the antigen binding domain comprises at least one of:
(i) a VH comprising a complementarity determining region (CDR) 1 comprising a sequence at least about 60%, at least 70%, at least 75%, at least 80%, at least 85%, at least 90%, at least 92%, at least 95%, at least 97%, at least 99% identical to the sequence set forth in SEQ ID NO: 6, a CDR2 comprising a sequence at least about 60%, at least 70%, at least 75%, at least 80%, at least 85%, at least 90%, at least 92%, at least 95%, at least 97%, at least 99% identical to the sequence set in SEQ ID NO: 7 and a CDR3 comprising a sequence at least about 60%, at least 70%, at least 75%, at least 80%, at least 85%, at least 90%, at least 92%, at least 95%, at least 97%, at least 99% identical to the sequence set forth in SEQ ID NO: 5;
(ii) a VH comprising a sequence at least about 80%, at least 85%, at least 90%, at least 92%, at least 95% at least 96%, at least 97%, at least 98%, at least 99% identical to the sequence set forth in SEQ ID NO: 92;
(iii) a VL comprising a CDR1 comprising a sequence at least about 60%, at least 70%, at least 75%, at least 80%, at least 85%, at least 90%, at least 92%, at least 95%, at least 97%, at least 99% identical to the sequence set forth in SEQ ID NO: 20, a CDR2 comprising the sequence at least about 65%, at least about 66%, at least 80%, at least 85%, at least 90%, at least 92%, at least 95%, at least 97%, at least 99% identical to the sequence set forth in SEQ ID NO: 18 and a CDR3 comprising a sequence at least about 60%, at least about 70%, at least about 75%, at least 80%, at least 85%, at least 90%, at least 92%, at least 95%, at least 97%, at least 99% identical to the sequence set forth in SEQ ID NO: 19;
(iv) a VL comprising a sequence at least about at least about 80%, at least 85%, at least 90%, at least 92%, at least 95% at least 96%, at least 97%, at least 98%, at least 99% identical to the sequence set forth in SEQ ID NO: 93;
(v) a VH comprising a CDR1 comprising the sequence set forth in SEQ ID NO: 6, a CDR2 comprising the sequence set forth in SEQ ID NO: 7 and a CDR3 comprising a sequence set forth in SEQ ID NO: 5;
(vi) a VH comprising the sequence set forth in SEQ ID NO: 92;
(vii) a VL comprising a CDR1 comprising the sequence set SEQ ID NO: 20, a CDR2 comprising the sequence set forth in SEQ ID NO: 18 and a CDR3 comprising the sequence set forth in SEQ ID NO: 19;
(viii) a VL comprising the sequence set forth in SEQ ID NO: 93;
(ix) a VH comprising a CDR1 comprising the sequence set forth in SEQ ID NO: 6, a CDR2 comprising the sequence set forth in SEQ ID NO: 7 and a CDR3 comprising the sequence set forth in SEQ ID NO: 5; and a VL comprising a CDR1 comprising the sequence set SEQ ID NO: 20, a CDR2 comprising a sequence set forth in SEQ ID NO: 18 and a CDR3 comprising the sequence set forth in SEQ ID NO: 19; or
(x) a VH comprising the sequence set forth in SEQ ID NO: 92 and a VL comprising the sequence set forth in SEQ ID NO: 93.

27. The antigen binding protein of any one of claims 1 to 26 wherein the protein is in the form of:
(i) a single chain Fv fragment (scFv);
(ii) a dimeric scFv (di-scFv);
(iii) one of (i) or (ii) linked to a constant region of an antibody, Fc or a heavy chain constant domain (CH) 2 and/or CH3, or
(iv) one of (i) or (ii) linked to a protein that binds to an immune effector cell;
(v) a diabody;
(vi) a triabody;
(vii) a tetrabody;
(viii) a Fab;
(ix) a F(ab′)2;
(x) a Fv;
(xi) one of (v) to (x) linked to a constant region of an antibody, Fc or a heavy chain constant domain (CH) 2 and/or CH3, or
(xii) one of (v) to (x) linked to a protein that binds to an immune effector cell.

28. The antigen binding protein of any one of claims 1 to 26, wherein the antigen binding protein is an antibody or antigen binding fragment thereof.

29. The antigen binding protein of claim 28, wherein the antigen binding protein is a monoclonal antibody, or fragment thereof, optionally a variable domain.

30. The antigen binding protein of any one of claims 5 to 8, wherein the protein comprises, consists essentially of or consists of an amino acid sequence of (in order of N to C terminus or C to N terminus):
SEQ ID NO: 88 and 89;
SEQ ID NO: 96 and 98;
SEQ ID NO: 97 and 98;
SEQ ID NO: 88 and 98;
SEQ ID NO: 96 and 89;
SEQ ID NO: 97 and 89;
SEQ ID NO: 48 and 55;
SEQ ID NO: 49 and 56;
SEQ ID NO: 50 and 57;
SEQ ID NO: 51 and 58;
SEQ ID NO: 52 and 56;
SEQ ID NO: 53 and 55;
SEQ ID NO: 54 and 59; or
SEQ ID NO: 92 and 93.

31. An antigen binding protein according to any one of claims 1 to 30, further comprising an Fc region, wherein the Fc region is engineered to have reduced capacity to induce antibody-dependent cell-mediated cytotoxicity (ADCC).

32. An antigen binding protein according to claim 31, wherein the Fc region is engineered to have reduced capacity to induce antibody-dependent cell-mediated cytotoxicity (ADCC) is mutated, deleted or modified at position 234, 235, and 331 as per SEQ ID NO:60 (where alanine is position 118) or at an equivalent position to 234, 235 and 331.

33. An antigen binding protein according to claim 32, wherein the mutations are L234F, L235E and P331S.

34. An antigen binding protein according to claim 32, wherein the Fc region comprises, consists essentially of or consists of an amino acid sequence shown in SEQ ID NO: 61.

35. An antigen binding protein according to any one of claims 1 to 30, further comprising an Fc region, wherein the Fc region is engineered to have an enhanced capacity to induce antibody-dependent cell-mediated cytotoxicity (ADCC).

36. An antigen binding protein according to claim 35, wherein the enhanced capacity to induce ADCC is conferred by mutation, deletion or modification of amino acids in the Fc region which interact with an Fc receptor.

37. An antigen binding protein according to claim 36, wherein the amino acids that are mutated, deleted or modified are at position 239, 330, and/or 332 as per SEQ ID NO: 60 (where alanine is position 118) or at an equivalent position to 239, 330 and/or 332, wherein preferably, the amino acids are mutated to S239D, A330L and I332E.

38. An antigen binding protein according to claim 37, wherein the Fc comprises, consists essentially of or consists of an amino acid sequence shown in SEQ ID NO: 62.

39. An antigen binding protein according to claim 9 or 10, wherein the Fc comprises, consists essentially of or consists of an amino acid sequence shown in SEQ ID NO: 62.

40. An antigen binding protein according to any one of claims 11 to 14, wherein the Fc comprises, consists essentially of or consists of an amino acid sequence shown in SEQ ID NO: 62.

41. An antigen binding protein according to any one of claims 1 to 40, wherein the antigen binding protein is in the form of an immunoglobulin variable domain, antibody, dab, scFv, Fab, Fab′, F(ab′)2, Fv fragment, diabody, triabody, linear antibody, single-chain antibody molecule, or multispecific antibody.

42. A nucleic acid encoding an antigen binding protein according to any one of claims 1 to 41.

43. A vector including a nucleic acid according to claim 42.

44. A cell including a vector according to claim 43 or nucleic acid according to claim 36.

45. A pharmaceutical composition including an antigen binding protein according to any one of claims 1 to 41 and a pharmaceutically acceptable carrier, diluent or excipient.

46. A method for treating or preventing a condition or disease associated with expression of CCR6, comprising administering to a subject in need thereof, an antigen binding protein according to any one of claims 1 to 41 or pharmaceutical composition according to claim 45, thereby treating or preventing the condition or disease associated with expression of CCR6 in the subject.

47. The method according to claim 46, wherein the disease or condition associated with expression of CCR6 is an inflammatory condition, autoimmune disease, infection, fibrosis or cancer, or pulmonary disorder.

48. The method according to claim 47, wherein the inflammatory condition is cardiovascular inflammation, gastrointestinal inflammation, hepatic inflammatory disorders, pulmonary inflammation, kidney inflammation, ocular inflammation, pancreatic inflammation, genitourinary inflammation, neuroinflammatory disorder, allergy, skeletal inflammation, inflammation caused by infection, inflammation caused from or in response to transplantation.

49. A method according to claim 47, wherein the autoimmune disease is Acquired Immunodeficiency Syndrome, alopecia areata, ankylosing, spondylitis, antiphospholipid syndrome, autoimmune Addison's disease, autoimmune haemolytic, anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune, dysfunction syndrome (CFIDS), chronic inflammatory demyelinating polyneuropathy (CIPD), cicatricial pemphigoid, cold agglutinin disease, crest syndrome, Crohn's disease, Degos' disease, dermatomyositis-juvenile, discoid lupus, essential mixed cryoglobulinemia, fibromyalgia fibromyositis, Graves' disease, Guillain-Barre syndrome, Hashimoto's thyroiditis, idiopathic pulmonary fibrosis, idiopathic thrombocytopenia purpura (ITP), IgA nephropathy, insulin dependent diabetes mellitus, juvenile chronic arthritis (Still's disease), juvenile rheumatoid arthritis, Meniere's disease, mixed connective tissue disease, multiple sclerosis, myasthenia gravis, pemacious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma (progressive systemic sclerosis (PSS), also known as systemic sclerosis (SS)), Sjogren's syndrome, stiff-man syndrome, systemic lupus erythematosus, Takayasu arteritis, temporal arteritis/giant cell arteritis, ulcerative colitis, uveitis, vitiligo or Wegener's granulomatosis.

50. A method according to claim 47 wherein the autoimmune disease is psoriasis.

51. A method according to claim 47, wherein the autoimmune disease is multiple sclerosis.

52. A method according to claim 47, wherein the fibrosis is Pulmonary fibrosis, Idiopathic pulmonary fibrosis, Cystic fibrosis, Cirrhosis, Endomyocardial fibrosis, Old myocardial infarction, Atrial Fibrosis, Mediastinal fibrosis, Myelofibrosis, Retroperitoneal fibrosis, Progressive massive fibrosis, Nephrogenic systemic fibrosis, Crohn's Disease, Keloid, Scleroderma/systemic sclerosis, Arthrofibrosis, Peyronie's disease, Dupuytren's contracture, or adhesive capsulitis.

53. A kit or article of manufacture including an antigen binding protein according to any one of claims 1 to 41, or a pharmaceutical composition according to claim 45.

55. Use of an antigen binding protein according to any one of claims 1 to 41 in the manufacture of a medicament for the treatment of an inflammatory condition, autoimmune disease, infection, fibrosis or cancer, pulmonary disorder or a condition or disease associated with expression of CCR6, preferably wherein the disease is an inflammatory or autoimmune disease.

55. An antigen binding protein according to any one of claims 1 to 41 for use in the treatment of an inflammatory condition, autoimmune disease, infection, fibrosis or cancer, pulmonary disorders or a condition or disease associated with expression of CCR6.

56. A use according to claim 54, or antigen binding protein for the use of claim 55 wherein the autoimmune disease is psoriasis.

57. A use according to claim 54, or antigen binding protein for the use of claim 55, wherein the autoimmune disease is multiple sclerosis.
